SEQUENCE LISTING (20 sequences)

SEQ ID NO: 1 - protein, 11 aa, Influenza B virus
GLFGAIAGFIE

SEQ ID NO: 2 - protein, 22 aa, Influenza B virus
MSLLTEVTPIRNEWGCRCNDSS

SEQ ID NO: 3 - protein, 6 aa, Mycobacterium tuberculosis
VTGMFA

SEQ ID NO: 4 - protein, 8 aa, Homo sapiens
DYKDDDDK

SEQ ID NO: 5 - protein, 18 aa, Unknown
  Description of Unknown Illustrative transduction peptide
SGEHTNGPSKTSVRWVWD

SEQ ID NO: 6 - protein, 18 aa, Unknown
  Description of Unknown Illustrative transduction peptide
SMTTMEFGHSMITPYKID

SEQ ID NO: 7 - protein, 18 aa, Unknown
  Description of Unknown Illustrative transduction peptide
QDGGTWHLVAYCAKSHRY

SEQ ID NO: 8 - protein, 18 aa, Unknown
  Description of Unknown Illustrative transduction peptide
MSDPNMNPGTLGSSHILW

SEQ ID NO: 9 - protein, 18 aa, Unknown
  Description of Unknown Illustrative transduction peptide
SPGNQSTGVIGTPSFSNH

SEQ ID NO: 10 - protein, 18 aa, Unknown
  Description of Unknown Illustrative transduction peptide
SSGANYFFNAIYDFLSNF

SEQ ID NO: 11 - protein, 18 aa, Unknown
  Description of Unknown Illustrative transduction peptide
GTSRANSYDNLLSETLTQ

SEQ ID NO: 12 - protein, 13 aa, Human Immunodeficiency virus
GRKKRRQRRRPPQ

SEQ ID NO: 13 - protein, 16 aa, Drosophila sp.
RQIKIWFQNRRMKWKK

SEQ ID NO: 14 - protein, 17 aa, Herpes simplex virus
NAKTRRHERRRKLAIER

SEQ ID NO: 15 - protein, 6 aa, Artificial Sequence
  Description of Artificial Sequence Synthetic peptide
RRRRRR

SEQ ID NO: 16 - protein, 4 aa, Artificial Sequence
  Description of Artificial Sequence Synthetic peptide
RGDT

SEQ ID NO: 17 - protein, 4 aa, Artificial Sequence
  Description of Artificial Sequence Synthetic peptide
DRVY

SEQ ID NO: 18 - protein, 5 aa, Artificial Sequence
  Description of Artificial Sequence Synthetic peptide
  MOD_RES (2)..(2): D-Ala
  MOD_RES (5)..(5): D-Met
YXGFX

SEQ ID NO: 19 - protein, 4 aa, Artificial Sequence
  Description of Artificial Sequence Synthetic peptide
RGDT

SEQ ID NO: 20 - protein, 4 aa, Artificial Sequence
  Description of Artificial Sequence Synthetic peptide
VIHN

(19) United States
(12) Patent Application Publication
(10) Pub. No.: US 2021/0106677 A1
(43) Pub. Date: Apr. 15, 2021

(54) VACCINE DEVELOPMENT METHODOLOGY BASED ON AN ADHESION MOLECULE

(71) Applicant: MatSfide, Inc., Livermore, CA (US)

(72) Inventor: Michael McElfresh, Livermore, CA (US)

(73) Assignee: MatSfide, Inc., Livermore, CA (US)

(21) Appl. No.: 16/826,529

(22) PCT Filed: Nov. 13, 2018
(86) PCT No.: PCT/US2018/060688

§ 371 Date: Mar. 23, 2020

Related U.S. Application Data
(60) Provisional application No. 62/585,261, filed on Nov. 13, 2017.

(51) Int. Cl. A61K 39/385

(52) U.S. Cl. CPC A61K 39/385; A61K 2039/605

(57) ABSTRACT

A method of eliciting immune responses using synthetic antigens involves generating a substitute antigen configured for a foreign molecule, generating a synthetic high affinity ligand molecule (SHAL) comprising at least one ligand configured to bind to an antigen presenting cell (APC) and at least one ligand specifically configured to bind with the substitute antigen, combining the SHAL with the substitute antigen through a chemical reaction forming an antigen presenting complex, introducing the antigen presenting complex to a user without an immune response to the foreign molecule.

### CROSS-REFERENCE TO RELATED APPLICATIONS

This application claims the benefit under 35 U.S.C. 119 to the U.S. application Ser. No. 62/585,261 filed on Nov. 13, 2017 which is incorporated herein by reference in its entirety.

### BACKGROUND

The Major Histocompatibility Complex (MHC) is a set of cell surface proteins essential for the acquired immune system to recognize foreign molecules in vertebrates. (MHC may refer to the set of genes or the proteins produced from those genes.) The main function of MHC molecules is to bind to antigens derived from a foreign molecule and display them on the cell surface for recognition by the appropriate T-cells. MHC molecules mediate interactions of leukocytes, also called white blood cells (WBCs), which are immune cells, with other leukocytes or with body cells. The human MHC is also called the HLA (Human Leukocyte antigen) complex (often just the HLA).

The MHC gene family is divided into three subgroups: class-I, class-II, and class-III. As shown in FIG. 1, MHC class-I occurs as an α chain composed of three domains—α₁, α₂, and α₃. The α₁ rests upon a unit of the non-MHC molecule (32 microglobulin. The α₃ domain is transmembrane, anchoring the MHC class-I molecule to the cell membrane. The peptide being presented is held by the floor of the peptide-binding groove, in the central region of the α₁/α₂ heterodimer (a molecule composed of two non-identical subunits). The genetically encoded and expressed sequence of amino acids of the peptide-binding groove's floor determines which particular peptide residues it binds.

In MHC class-I, any nucleated cell normally presents cytosolic peptides, mostly self-peptides derived from protein turnover and defective ribosomal products. During viral infection, intracellular microorganism infection, or cancerous transformation, such proteins degraded in the proteasome are loaded onto MHC class-I molecules and displayed on the cell surface. T-lymphocytes may detect a peptide displayed at 0.1%-1% of the MHC molecules.

As shown in FIG. 2, MHC Class-II occurs as two chains, α and β, each having two domains—α₁ and α₂ and β₁ and β₂—each chain having a transmembrane domain, α₂ and β₂, respectively, anchoring the MHC Class-II molecule to the cell membrane. The peptide-binding groove is formed of the heterodimer of α₁ and β₁.

FIG. 3. Schematic diagram of HLA-DR, a class II MHC molecule, complexed with the antigen interacting with the T-cell receptor in order to identify if the peptide antigen is foreign.

The SH7139 Molecule

A molecule with attributes required to serve as an adhesion molecule for the process described below, is SH7139, a molecule that was designed to treat B-cell lymphoma. It binds strongly and specifically to the β-subunit of HLA-DR molecules that contain an amino-acid sequence similar to the DRB1*10 epitope. SH7139 binds in the region where antigens would normally bind. It was designed to a carry a lethal targeting atom by using a chelating agent (e.g., DotA (1,4,7,10-Tetraazacyclododecane-1,4,7,10-tetraacetic acid)) that is chemically bonded to SH7139 via a polymer tether. (SH7139 may refer to the molecule that has DotA incorporated or some other variants that have chemical moieties other than DotA.) The cancer treatment strategy assumes that HLA-DR is over-expressed on B-cell lymphoma cells, so by attaching a radioactive atom, for example, to the DotA moiety on SH7139, the complex of radioactive targeting atom with SH7139, when injected, would bind strongly to the HLA-DR molecules and the radioactive decay of the bound atoms would kill the cells containing HLA-DR. The design of SH7139 is such that the DotA moiety may be replaced with a range of alternative molecules. SH7139 is thought to also have other mechanisms for killing cancer cells without the use of the radioactive atom. SH7139 has been shown to bind to canine tissues.

Vaccines

A vaccine is a biological preparation that provides active acquired immunity to a particular disease. A vaccine typically contains an agent that resembles a portion of a disease-causing microorganism and is often made from weakened or killed forms of the microbe, its toxins, or one of its surface proteins. The agent stimulates the body's immune system to recognize the agent as a threat, destroy it, and recognize and destroy any of these microorganisms or foreign molecules that it later encounters. Vaccines may be prophylactic (example: to prevent or ameliorate the effects of a future infection by a natural or “wild” pathogen), or therapeutic (e.g., vaccines against cancer are being investigated). Vaccines utilize the MHC molecules and associated system described above for developing immunity.

The natural immune system has various limitations. While the number is quite large, there are a limited number of molecules for which the immune system may develop a response. In some cases, vaccines are made from inactivated toxic compounds that cause illness rather than the micro-organism producing the toxin. Examples of toxoid-based vaccines include tetanus and diphtheria. Toxoid vaccines are known for their efficacy. Not all toxoids are for micro-organisms; for example, Crotalus atrox toxoid is used to vaccinate dogs against rattlesnake bites.

Rather than introducing an inactivated or attenuated micro-organism to an immune system (which would constitute a “whole-agent” vaccine), a fragment of it may create an immune response. Examples include the subunit vaccine against Hepatitis B virus that is composed of only the surface proteins of the virus (previously extracted from the blood serum of chronically infected patients, but now produced by recombination of the viral genes into yeast), the virus-like particle (VLP) vaccine against human papillomavirus (HPV) that is composed of the viral major capsid protein, and the hemagglutinin and neuraminidase subunits of the influenza virus. Subunit vaccine is being used for plague immunization.

Certain bacteria have polysaccharide outer coats that are poorly immunogenic. By linking these outer coats to proteins (e.g., toxins), the immune system may be led to recognize the polysaccharide as if it were a protein antigen. This approach is used in the Haemophilus influenzae type B vaccine.

Currently vaccines are being designed to stimulate humoral and/or cell mediated immunity. Attempts are being made to develop vaccines to help cure chronic infections, as opposed to preventing disease. Vaccines are being developed to defend against bioterrorist attacks such as anthrax, plague, and smallpox. Appreciation for sex and pregnancy differences in vaccine responses might change the strategies used by public health officials. Scientists are now trying to develop synthetic vaccines by reconstructing the outside structure of a virus, this will help prevent vaccine resistance.

Principles that govern the immune response may now be used in tailor-made vaccines against many noninfectious human diseases, such as cancers and autoimmune disorders. For example, the experimental vaccine CYT006-AngQb has been investigated as a possible treatment for high blood pressure. Factors that have an impact on the trends of vaccine development include progress in translatory medicine, demographics, regulatory science, political, cultural, and social responses.

### BRIEF SUMMARY

Presented here is a method for commandeering the immune system in order to produce an immune response to a wider range of antigens (foreign bodies) than the system would be able to produce under natural/normal conditions. The process involves an adhesion molecule to attach either synthetic, or naturally-occurring, antigens to the binding site of naturally-occurring Major Histocompatibility Complex (MHC) molecules; including those MHC proteins that are receptors on antigen Presenting Cells (APCs), e.g., macrophages, B-cells, or dendritic cells. In order to utilize features of the body's normal immune system, the chemically-synthetized adhesion molecule is designed to bind selectively and strongly at, or near, the site on MHC molecules where a specific natural antigen would naturally bind. The substitute antigen is usually attached to the adhesion molecule using a chemical bond prior to attaching/introducing the substitute-antigen/adhesion-molecule complex (SAAMC) to the MHC molecules; this introduction may be done either in-vivo or in-vitro. The process described here allows any antigen, of essentially any composition, to be bound to any APC receptor molecule using the adhesion molecule, thereby overriding the normal bonding mechanism which involves the APC receptor molecules binding weakly to a limited set of natural antigens. After the SAAMC is bound to the APC receptor molecule on an antigen presenting cell, the larger complex of APC plus SAAMC is introduced to the natural immune system in order to elicit a normal immune response. The fact that any antigen may be attached to essentially any MHC molecule is what makes this a vaccine that may be universally applied for developing an immune response. Here synthetic refers to the fact that the adhesion molecule is a chemically-synthesized small-molecule and substitute antigens may also be chemically synthesized molecules rather than naturally occurring.

The strong binding of the antigen to MHCs using the adhesion molecule translates into larger amounts of time for the critical interactions of APCs with T-cells to take place. This should, in turn, translate into smaller doses of vaccine. This method also circumvents the early steps (e.g., phagocytosis and endocytosis of the foreign/invading entity) in the natural immune response that is responsible for generating the antigens (that normally bind to APCs) and allows an immune response to take place much more rapidly

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an MHC Class-I 100 in accordance with one embodiment.

FIG. 2 illustrates an MHC Class-II 200 in accordance with one embodiment.

FIG. 3 illustrates a diagram 300 in accordance with one embodiment.

FIG. 4 illustrates a diagram 400 in accordance with one embodiment.

FIG. 5 illustrates a diagram 500 in accordance with one embodiment.

FIG. 6 illustrates a diagram 600 in accordance with one embodiment.

FIG. 7 illustrates a diagram 700 in accordance with one embodiment.

FIG. 8 illustrates a diagram 800 in accordance with one embodiment.

FIG. 9 illustrates a diagram 900 in accordance with one embodiment.

FIG. 10 illustrates a diagram 1000 in accordance with one embodiment.

FIG. 11 illustrates a method 1100 in accordance with one embodiment.

### DETAILED DESCRIPTION

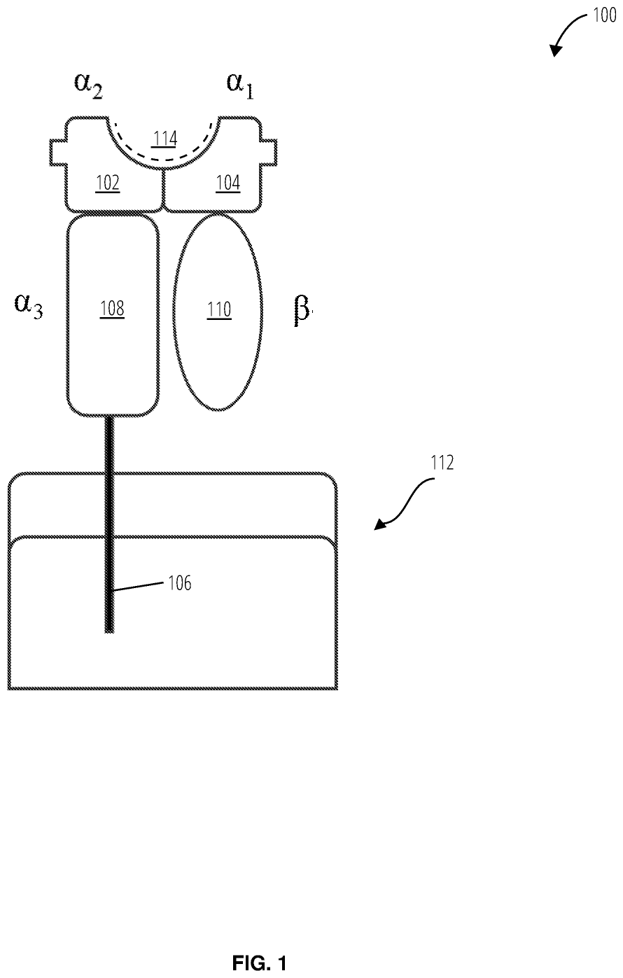

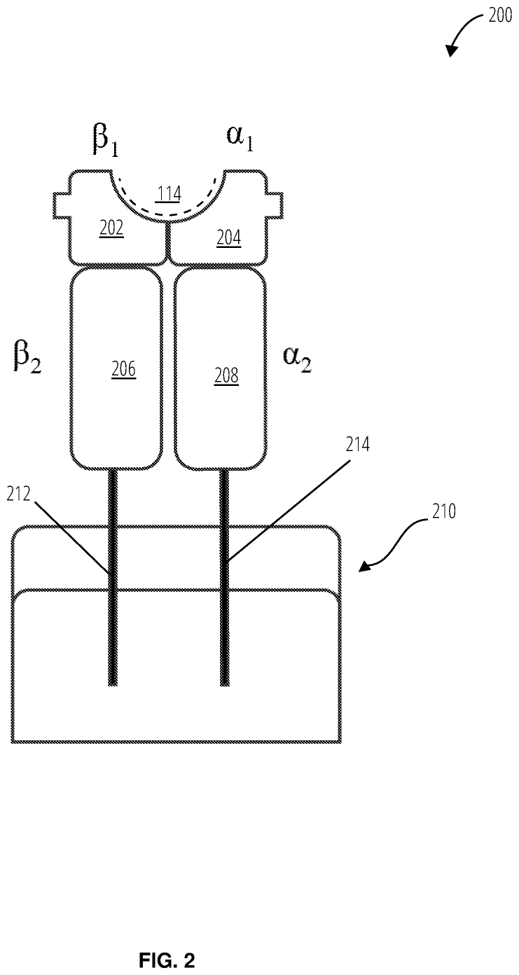

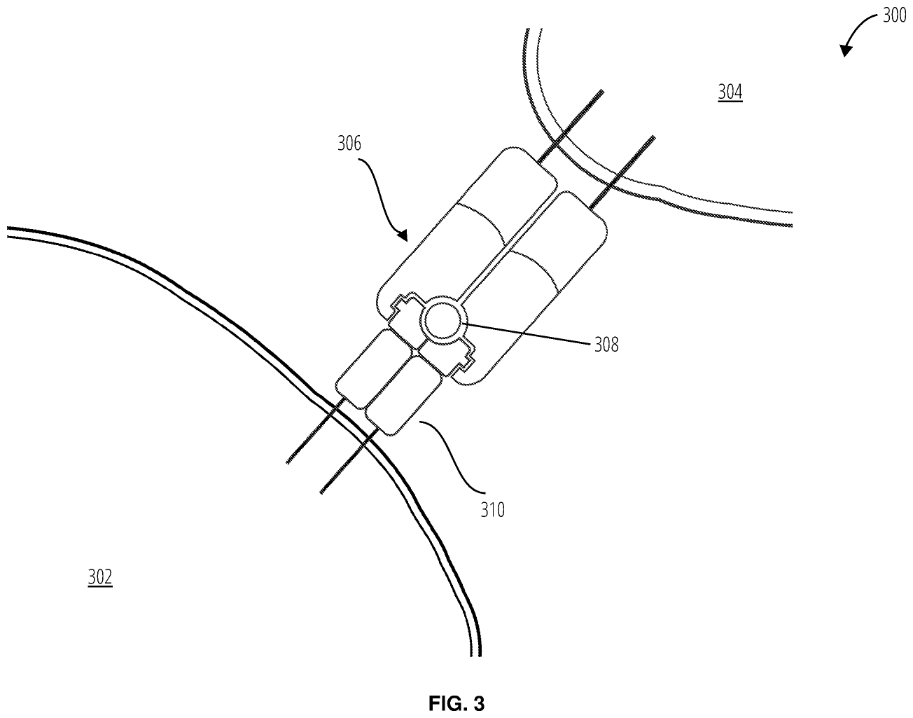

FIG. 1 shows a schematic diagram of class-I MHC receptor molecules with part of subunit a₃ is anchored in the membrane of an antigen presenting complex (APC) molecule. FIG. 2 shows a schematic diagram of class-II MHC receptor molecules with part of subunits α₂ and β₂ anchored in the membrane of the APC molecule. FIG. 3 shows the two sub-units, a and β, of HLA-DR, which is a class-II MHC receptor, forming a dimer on the cell surface with each subunit attached to the membrane. There is a region (a slot) of the receptor that binds peptide antigens for presentation to T-helper cells.

In the case of MHC Class-II, phagocytes such as macrophages and immature dendritic cells take up entities by phagocytosis into phagosomes—though B-cells exhibit the more general endocytosis into endosomes—which fuse with lysosomes whose acidic enzymes cleave the ingested protein into many different peptides. Due in part to the physicochemical dynamics in molecular interaction with the particular MHC Class-II variants borne by the host and encoded in the host's genome, a particular peptide exhibits immune-dominance and loads onto MHC Class-II molecules. These are trafficked to and externalized on the cell surface.

Diversity of antigen presentation, mediated by MHC classes-I and II, is attained in at least three ways: (1) an organism's MHC repertoire is polygenic (via multiple, interacting genes); (2) MHC expression is codominant (from both sets of inherited alleles); (3) MHC gene variants are highly polymorphic (diversely varying from organism to organism within a species).

An important step in the development of a long-lived immune response is the presentation to T-cells of antigens (i.e. foreign entities) that bind to antigen presenting cell (APC) receptors like HLA-DR (Human Leukocyte antigen-antigen D Related) or other particular molecules that are part of the MHC. For example, each person has a fairly large number of different HLA-DR molecules associated with the diversity naturally produced, however there is a finite limit to the number of different naturally-derived binding sites for antigens.

The function of the APC receptors involved in the immune response of humans (and other vertebrates) is to present peptide antigens, potentially foreign in origin, to the immune system for the of purpose activating T-(helper)-cells into T-effector cells that, in some cases, leads to the production of antibodies against the same peptide found in the antigen. Antigen presenting cells (APCs) are the cells in which MHC receptor molecules are commonly found. For instance, the complex of HLA-DR and its ligand (i.e. the antigen), a peptide of nine amino acids in length or longer, constitutes a natural ligand for the T-cell receptor. In the instance of an infection, the antigen is bound into an HLA-DR molecule and presented to a few of a great many, but finite number of, T-cell receptors found on T-helper cells. These cells then bind to corresponding antigens on the surface of particular B-cells stimulating B-cell proliferation.

Shown schematically in FIG. 3, T-cells require presentation via major MHC molecules (e.g. HLA-DR) to recognize foreign antigens—a requirement known as MHC restriction. These cells have receptors that are similar to B-cell receptors, and each cell recognizes only a few class-II/peptide combinations. Once a T-cell recognizes a peptide within an MHC Class-II molecule, it may stimulate B-cells that also recognize the same molecule in their B-cell receptors. Thus, T-cells help B-cells make antibodies to the same foreign antigens. Each HLA may bind many peptides, and each person has 3 HLA types and may have 4 isoforms of DP, 4 isoforms of DQ, and 4 isoforms of DR (2 of DRB1, and 2 of DRB3, DRB4, or DRB5) for a total of 12 isoforms.

A molecule with many of the attributes required to serve as an adhesion molecule for the process described below, is SH7139, a molecule that was designed to treat B-cell lymphoma. It binds strongly and specifically to the β-subunit of HLA-DR molecules that contain an amino-acid sequence similar to the DRB1*10 epitope. SH7139 binds in the region where antigens would normally bind. It was designed to a carry a lethal targeting atom by using a chelating agent (e.g., DotA) that is chemically bonded to SH7139 via a polymer tether. (SH7139 may refer to the molecule that has DotA incorporated or some other variants that have chemical moieties other than DotA.) The cancer treatment strategy assumes that HLA-DR is over-expressed on B-cell lymphoma cells. By attaching a radioactive atom, for example, via the DotA moiety to SH7139, the complex of radioactive targeting atom with SH7139, when injected, would bind strongly to the HLA-DR molecules and the radioactive decay of the bound atoms would kill the cells containing HLA-DR. The design of SH7139 is such that the DotA moiety may be replaced with a range of alternative molecules. SH7139 is thought to also have other mechanisms for killing cancer cells without the use of the radioactive atom. SH7139 has been shown to bind to canine tissues.

Vaccine Implementation

A vaccine is a biological preparation that provides active acquired immunity to a particular disease. A vaccine typically contains an agent that resembles a disease-causing microorganism and is often made from weakened or killed forms of the microbe, its toxins, or one of its surface proteins. The agent stimulates the body's immune system to recognize the agent as a threat, destroy it, and recognize and destroy any of these microorganisms that it later encounters. Vaccines may be prophylactic (example: to prevent or ameliorate the effects of a future infection by a natural or “wild” pathogen), or therapeutic (e.g., vaccines against cancer are being investigated). Vaccines utilize the MHC molecules and associated system described above for developing immunity.

The natural immune system has various limitations. While the number is quite large, there are a limited number of molecules for which the immune system may develop a response. In some cases, vaccines are made from inactivated toxic compounds that cause illness rather than the micro-organism. Examples of toxoid-based vaccines include tetanus and diphtheria. Toxoid vaccines are known for their efficacy. Not all toxoids are for micro-organisms; for example, Crotalus atrox toxoid is used to vaccinate dogs against rattlesnake bites.

Rather than introducing an inactivated or attenuated micro-organism to an immune system (which would constitute a “whole-agent” vaccine), a fragment of it may create an immune response. Examples include the subunit vaccine against Hepatitis B virus that is composed of only the surface proteins of the virus (previously extracted from the blood serum of chronically infected patients, but now produced by recombination of the viral genes into yeast), the virus-like particle (VLP) vaccine against human papillomavirus (HPV) that is composed of the viral major capsid protein, and the hemagglutinin and neuraminidase subunits of the influenza virus. Subunit vaccine is being used for plague immunization.

Certain bacteria have polysaccharide outer coats that are poorly immunogenic. By linking these outer coats to proteins (e.g., toxins), the immune system may be led to recognize the polysaccharide as if it were a protein antigen. This approach is used in the Haemophilus influenzae type B vaccine.

Currently vaccines are being designed to stimulate innate immune responses, as well as adaptive. Attempts are being made to develop vaccines to help cure chronic infections, as opposed to preventing disease. Vaccines are being developed to defend against bioterrorist attacks such as anthrax, plague, and smallpox. Appreciation for sex and pregnancy differences in vaccine responses might change the strategies used by public health officials. Scientists are now trying to develop synthetic vaccines by reconstructing the outside structure of a virus, this may help prevent vaccine resistance.

Principles that govern the immune response may now be used in tailor-made vaccines against many noninfectious human diseases, such as cancers and autoimmune disorders. For example, the experimental vaccine CYT006-AngQb has been investigated as a possible treatment for high blood pressure. Factors that have an impact on the trends of vaccine development include progress in translatory medicine, demographics, regulatory science, political, cultural, and social responses.

Described here is a method for strongly bonding essentially any antigen to Major Histocompatibility Complex (MHC) receptor molecules in an artificial manner that constitutes a universal mechanism for production of vaccines. Antigens that would not otherwise bind to existing MHC receptors, i.e. substitute antigens, may be attached to a synthetic molecule, i.e. adhesion-molecule, which is specifically designed to strongly bind to a location on, or near, the region of the MHC receptor where antigens are naturally bound on MHC Antigen Presenting Cells (APCs). The adhesion molecule serves to anchor any potential (substitute) antigen to the APC receptor and allows positioning of that antigen for presentation to the T-cells in order to initiate an immune response from the body.

The process described here uses a small synthetic molecule for the adhesion molecule, that is designed to hold an artificially selected antigen (i.e. Substitute antigen)—rather than the antigen to which the MHC molecule is naturally inclined to bond—in the slot-like binding site of an MHC-APC molecule. The process uses a small adhesion molecule that, on the one hand, binds strongly to the APC receptor but can, at the same time, also be strongly bonded, using a chemical reaction, to the artificially selected antigen (i.e. Substitute antigen). A molecule that has some of the attributes required for an adhesion molecule is SH7139, which bonds strongly and selectively to the antigen binding site of HLA-DR10 (in particular, the DRB10*1 epitope). In addition to its HLA binding region, SH7139 carries a DotA moiety tethered to the binding region. This DotA moiety, however, may be replaced during SH7139 synthesis with the substitute antigen. In one configuration of the invention, SH7139 may be synthesized in a modified form with the DotA moiety replaced with the appropriate substitute antigen attached to the tether using a carbodiimide chemistry reaction.

The vaccine (i.e. the SAAMC/antigen presenting complex) is produced by attaching the substitute-antigen to the adhesion-molecule via a strong bond using a chemical synthetic method (for example, by carbodiimide chemistry). This vaccine, which consists of a molecular complex consisting of the substitute-antigen chemically bound to the adhesion-molecule, is introduced to APCs either in vivo or in vitro. The treated APCs then use the body's machinery to develop an immune response. This method allows for development of an immune response well in advance of exposure [prophylactic vaccine] but is different from the natural immune process in that it skips earlier steps that would normally take place in the body in order to develop an immune response, e.g. phagocytosis or endocytosis and the associated processing to produce a natural antigen. The process described here allows for presentation of antigens that do not occur naturally and allows the presentation of the antigen to the T-cell in a spatial orientation or form that would not occur naturally that may be more preferential for developing an immune response.

A method of eliciting immune responses using synthetic antigens may involve generating at least one substitute antigen from a foreign molecule (e.g., pathogen, toxicant, etc.). The method may then generate a synthetic high affinity ligand molecule (SHAL) comprising at least one ligand configured to bind to an antigen presenting cell (APC) and at least one attachment molecule specifically configured to bind with the substitute antigen. The method may then combine the SHAL with the substitute antigen through a chemical reaction forming an antigen presenting complex by way of at least one attachment molecule. The method may then introduce the antigen presenting complex to a user without an immune response to the foreign molecule (e.g., pathogen, toxicant, etc.). The method may then measure the immune response in the user for the substitute antigen.

In some instances, the substitute antigen may be generated from the toxicant, such as the fragment of a surface protein or surface molecules associated with the toxicant.

In the method of eliciting immune responses using synthetic antigens, the method may further involve generating the substitute antigen from the pathogen by extracting at least a subcellular fragment from the pathogen.

In the method of eliciting immune responses using synthetic antigens, the pathogen may be a virus or virus like organism, such as a prion but may include a viral fragment with infectious potential. Additionally, the pathogen may be a microorganism, such as a bacterium, protozoa, or fungal organism.

The antigen presenting complex may be configured to position the substitute antigen between a peptide-binding groove of a major histocompatibility complex (MHC) Class-II molecule and a T-cell receptor protein. In some configurations, the at least one ligand may be configured to bind to the APC through a peptide-binding groove of an MHC Class-II molecule. Specifically, the at least one ligand may be configured to bind to the APC at β₁ domain of the peptide-binding groove of the MHC Class-II molecule.

In some configurations, the at least one ligand comprises three ligands configured to bind to the APC through the β₁ domain of the peptide-binding groove of the MHC Class-II molecule.

In some configurations, the at least one ligand comprises at least two ligands configured to bind to the APC between an MHC Class-II molecule and the T-Cell receptor protein.

In some configurations, a first ligand of the at least two ligands binds to a peptide-binding groove of the MHC Class-II molecule on a β₁ domain and a second ligand of the at least two ligands binds to the α₁ domain of the peptide-binding groove of the MHC Class-II molecule.

In some configurations, a first ligand of the at least two ligands binds to a peptide-binding groove of the MHC Class-II molecule on a β₁ domain and a second ligand of the at least two ligands binds to the β₁ domain of the MHC Class-II molecule adjacent to the peptide-binding groove.

In some configurations, SHAL comprises at least two linkage compounds specifically configured to bind with the substitute antigen.

In some configurations, the method of eliciting immune responses using a synthetic antigen further comprises generating the substitute antigen from the pathogen or the toxicant may involve selecting at least one version of the substitute antigen for generating the antigen presenting complex.

In some configurations, the method of eliciting an immune response using a synthetic antigen further comprises measuring the immune response in the user for the substitute antigen may involve measuring a corresponding antibody for the substitute antigen in the user, and introducing a new antigen presenting complex comprising a variant substitute antigen, in response to the corresponding antibody being below a minimum threshold. Furthermore, measurement of the immune response may be utilized in the development and improvement of the substitute antigens for the purpose of identifying antigen presenting complexes with broad population and/or multi species application, as well as tailoring the substitute antigen-adhesion molecule complex for specific individuals.

The substitute antigens may, in principle, be any kind of molecule, not only amino acid chains. However, amino acid (AA) chains may provide a starting point for substitute antigens which may be 6-9 AAs in length. In some configurations, the small amino acid chain utilized as the substitute antigen may be larger than 9 amino acids in length.

All AAs have least two functional groups, an amide group and a carboxylic acid group. Peptide bonds are used in vivo to bond together AAs into proteins. This involves the reaction of the amide of one AA with the carboxylic acid of the adjacent AA. A complete AA chain has terminal ends of an amide on one end and a carboxylic acid on the other. Either of these terminal ends may be used for attaching to the adhesion molecule (via a peptide bond or some other kind of bond).

In the case of SHAL7139, carbodiimide chemistry is used to make a peptide of bond between the fourth tether on SH7139 and the DotA molecule that is attached in one of the variations of SH7139.

In addition to amide and a carboxylic acid groups, AAs have wide range other functional groups associated with the side-chains of AAs that are attached to the central carbon atom of the AA. In eukaryotes, there are 21 proteinogenic amino acids with different side chains.

A wide range of chemistries are available for making peptide bonds between amide and carbonyl groups, and for linking to the side chains. One possible way to group these chemistries are under the headings Active esters/Additives, Carbodiimides Carbonyldiimidazole Derivatives Phosphonium/Uronium/Formamidinium, and types of peptide couplings. This is not an exhaustive list but represents a sample of the chemistries for which the reagents are commercially available off of the shelf.

The attachment between the adhesion molecule and a peptide-chain substitute-antigen may be at either one of the terminal ends of the peptide chain or it may be with a side-chain of one of the AAs in the peptide chain. By selecting which AA side-chain in the sequence is binding to the adhesion-molecule tether, the orientation of the substitute-antigen for presentation to the T-cell may be altered. This variable may allow optimization of the immune response to the substitute antigen.

A method of eliciting immune responses using synthetic antigens involves generating at least one substitute antigen configured for a foreign molecule (e.g., pathogen, toxicant, etc.). The method generates a synthetic high affinity ligand molecule (SHAL) comprising at least one ligand configured to bind to an antigen presenting cell (APC) and at least one adhesion molecule specifically configured to bind with the substitute antigen. The method combines the SHAL with the substitute antigen through a chemical reaction forming an antigen presenting complex by way of the at least one adhesion molecule. The method introduces the antigen presenting complex to a user without an immune response to the foreign molecule (e.g., pathogen, toxicant, etc.). The method measures the immune response in the user for the substitute antigen.

In some configurations, the substitute antigen is generated from the toxicant, the substitute antigen being a fragment of a surface protein associated with the toxicant.

In some configurations, the method generates the substitute antigen from the pathogen. The method also extracts at least a subcellular fragment from the pathogen to generate the substitute antigen. The pathogen may be a virus or virus like organism. The pathogen may be a microorganism.

In some configurations, the antigen presenting complex is configured to position the substitute antigen between a peptide-binding groove of a major histocompatibility complex (MHC) Class-II molecule and a T-cell receptor protein.

In some configurations, the at least one ligand is configured to bind to the APC through a peptide-binding groove of an MHC Class-II molecule. In some configurations, the at least one ligand is configured to bind to the APC at β₁ domain of the peptide-binding groove of the MHC Class-II molecule. In some configurations, the at least one ligand comprises three ligands configured to bind to the APC through at the β₁ domain of the peptide-binding groove of the MHC Class-II molecule.

The at least one ligand may comprise at least two ligands configured to bind to the APC between an MHC Class-II molecule and the T-Cell receptor protein. In some configurations, a first ligand of the at least two ligands may bind to a peptide-binding groove of the MHC Class-II molecule on a β₁ domain and a second ligand of the at least two ligands binds to the α₁ domain of the peptide-binding groove of the MHC Class-II molecule. In some configurations the first ligand of the at least two ligands binds to a peptide-binding groove of the MHC Class-II molecule on a β₁ domain and a second ligand of the at least two ligands binds to the β₁ domain of the MHC Class-II molecule adjacent to the peptide-binding groove.

The SHAL may comprise at least two linkage compounds specifically configured to bind with the substitute antigen.

In some configurations, the method may involve generating the substitute antigen from the pathogen or the toxicant.

In some configurations, the method measures the immune response in the user for the substitute antigen involves measuring a corresponding antibody for the substitute antigen in the user, and introducing a new antigen presenting complex comprising a variant substitute antigen, in response to the corresponding antibody being below a minimum threshold.

In some configurations, the method may generate the substitute antigen further involves selecting at least one version of the substitute antigen for generating the antigen presenting complex.

In some configurations, the at least one substitute antigen may be configured for the pathogen or the toxicant further comprises a synthetic molecule configured for a pathogen or a toxicant.

Referencing FIG. 1, a schematic diagram of a major histocompatibility complex Class-1 (MHC Class-I 100) molecule is shown. The MHC Class-I 100 includes an α₂ domain 102, an α₁ domain 104, and an α₃ domain 108, with a β₂ microglobulin 110 domain positioned between the α₁ domain 104 and the cell membrane 112. The α₃ domain 108 includes an α₃ domain transmembrane anchor 106 that retains the MHC Class-I 100 to the cell membrane 112. The α₂ domain 102 and the α₁ domain 104 form a peptide-binding groove 114 for binding an antigen for presentation to a T-Cell.

Referencing FIG. 2, a schematic diagram of a major histocompatibility complex Class-II (MHC Class-II 200) is shown. The MHC Class-II 200 includes a β₁ domain 202, a β₂ domain 206, an α₁ domain 204, and an α₂ domain 208. The MHC Class-II 200 is retained to a cell membrane 210 of certain cells, (e.g., macrophages, B-cells, dendritic cells,) through a β₂ transmembrane anchor 212 and an α₂ transmembrane anchor 214. A peptide-binding groove 114 is formed between the β₁ domain 202 and the α₁ domain 204 and retains an antigen.

Referencing FIG. 3, a schematic diagram 300 shows the interaction between an antigen presenting cell 302 and a T-cell 304. The major histocompatibility complex 310 of the antigen presenting cell 302 engages a corresponding T-cell receptor 306 of the T-cell 304 activating the T-cell 304 into an effector T-Cell.

The T-cell 304 is a cluster of differentiation 4 cells (CD4) cells or helper T cells that provide protection against different pathogens. The T-cell 304 is a naive T cell that has yet to encounter an antigen, but once coupled to an antigen presenting cell (APC), the T-cell 304 is converted into activated effector T cell. These APCs, such as macrophages, dendritic cells, and B cells in some circumstances, load antigenic peptides onto the MHC of the cell, in turn presenting the peptide to receptors on T cells. The most important of these APCs are highly specialized dendritic cells; conceivably operating solely to ingest and present antigens.

Activated Effector T cells may be organized into three functioning classes, detecting peptide antigens originating from various types of pathogen: The first class being Cytotoxic T cells, which kill infected target cells by apoptosis without using cytokines, the second class being T-Helper (TH1) cells, which primarily function to activate macrophages, and the third class being T-Helper 2 (TH2) cells, which primarily function to stimulate B cells into producing antibodies.

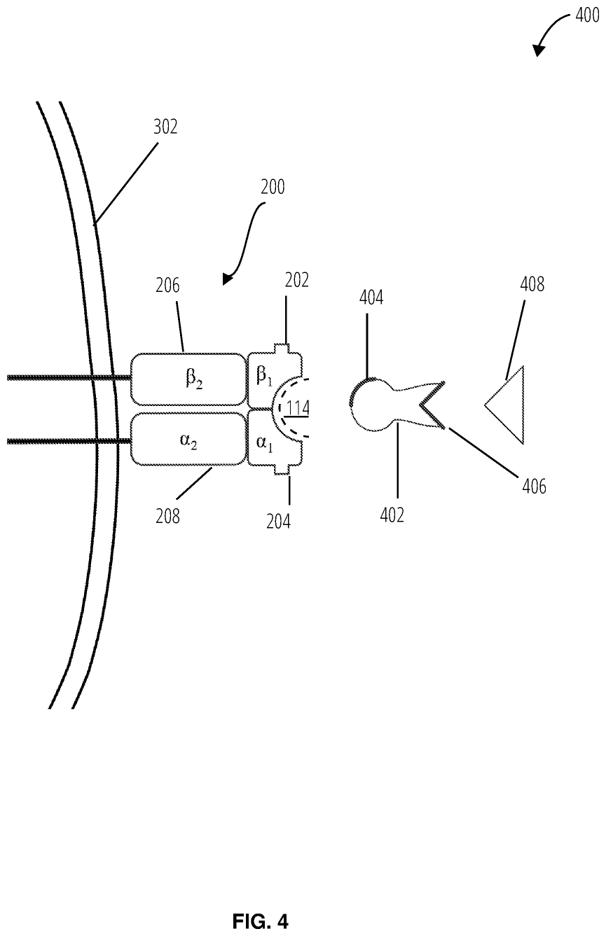

Referencing FIG. 4, a schematic diagram 400 shows a generalized process by which a SHAL 402 attaches a substitute antigen 408 to an MHC Class-II 200 on an antigen presenting cell 302. The SHAL 402 comprises at least one ligand binding site 404 configured to bind with the peptide-binding groove 114 and a linkage 406 configured to bind with the substitute antigen 408. Specifically, the at least one ligand binding site 404 is configured to bind with the β₁ domain 202 in the peptide-binding groove 114 of the MHC Class-II 200. The linkage 406 is configured to bind with the substitute antigen 408.

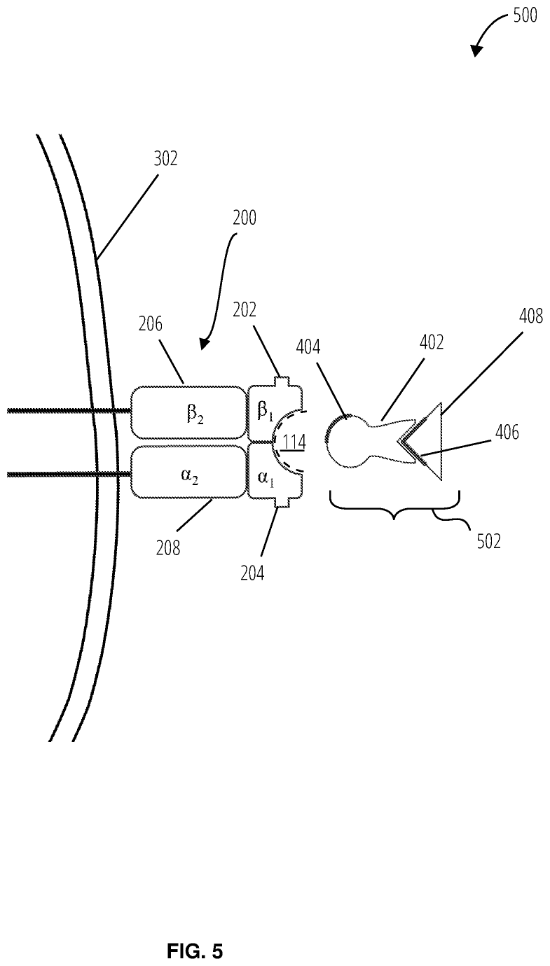

Shown in FIGS. 5-7, are a set of simplified schematic diagrams showing one way this vaccine production process may operate. FIG. 4 shows the three key elements, (1) the naturally-occurring HLA-DR receptor (equivalently the MHC or APC receptor) antigen binding site (or slot), (2) the synthetic adhesion-molecule, and (3) the substitute-antigen of interest. The adhesion molecule is a linker/tether that is used for attaching the substitute antigen to the adhesion molecule using a chemical reaction. The HLA-DR binding site is an integral part of the natural HLA-DR dimer protein found on the surface of APCs (e.g., macrophages, B-cells, and dendritic cells).

Referencing FIG. 5, a schematic diagram 500 shows the SHAL 402 combined with the substitute antigen 408 through the linkage 406 forming a SHAL substitute antigen complex 502.

FIG. 5 shows the adhesion molecule bonded to the antigen via the linker. This adhesion-molecule/antigen complex (SAAMC) may be usable as synthetic vaccine in essentially this form. Here the substitute antigen may be covalently bonded to the adhesion molecule using carbodiimide chemistry, but there are many different chemistries that may be used for binding the two components of the complex together that may depend on the functional groups on the molecules being chemically bonded. The SAAMC may be usable as a synthetic vaccine in this form. It may be introduced to the host by intravenous, intramuscular, or intraperitoneal injection, or orally, in an isotonic saline solution. The addition of a co-solvent, as for example, DMSO may help improve solubility. Alternatively, blood may be removed from the individual, and then, with or without cell separation, treated with the vaccine, and then reinjected after proper incubation in vitro with the vaccine.

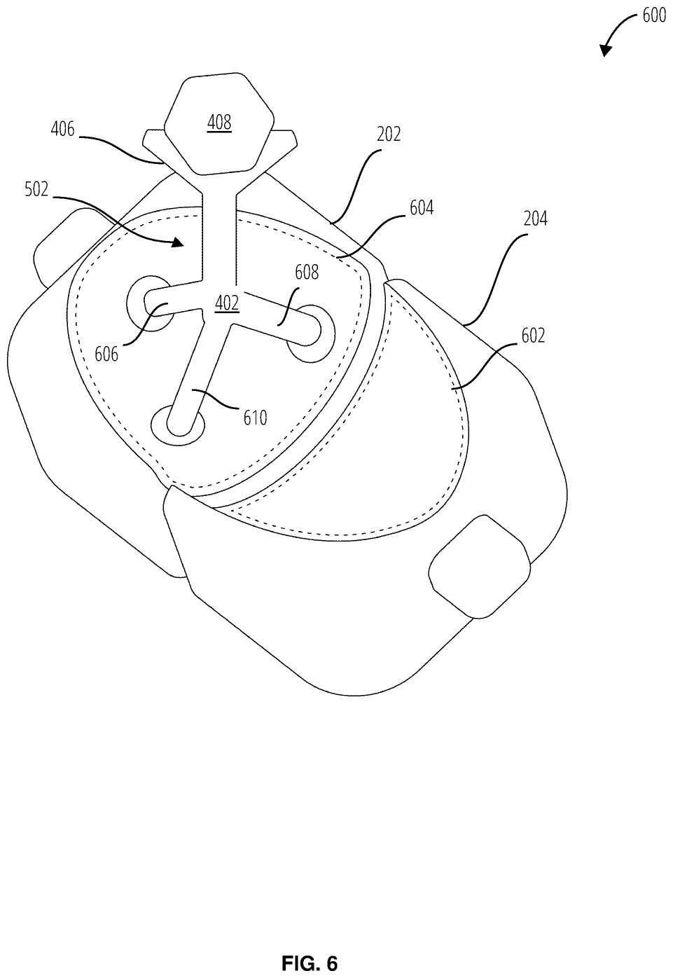

FIG. 6 illustrates a diagram 600 showing the peptide-binding groove of the MHC Class-II molecule with the SHAL substitute antigen complex 502 attached. The SHAL substitute antigen complex 502 comprises three ligands (a first ligand 606, a second ligand 610, and a third ligand 608). In one configuration of the SHAL 402, the three ligands are configured to bind to the β₁ domain binding region 604 of the peptide-binding groove that sits opposite the α₁ domain binding region 602 on the MHC Class-II molecule.

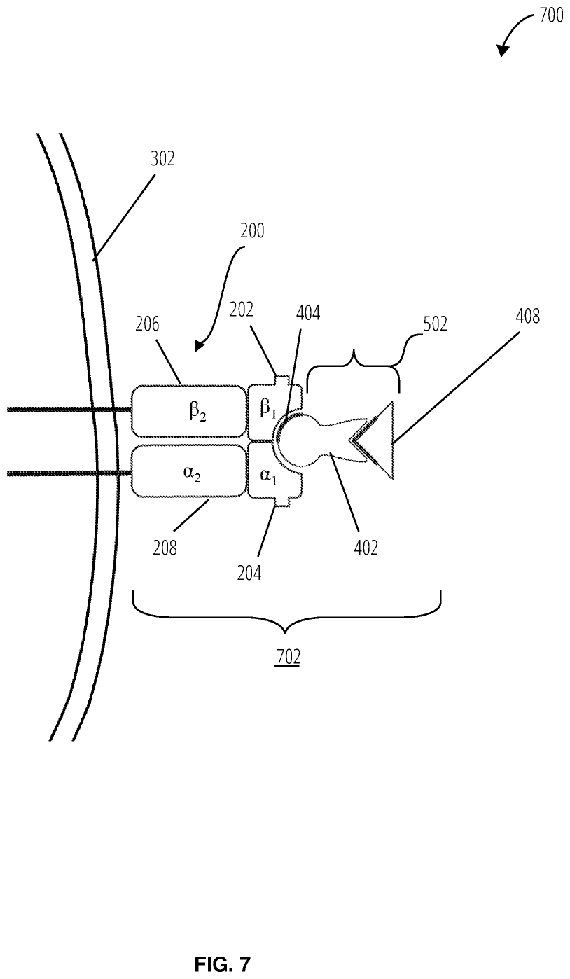

Referencing FIG. 7, a schematic diagram 700 shows the SHAL substitute antigen complex 602 bound to the MHC Class-II 200 through the β₁ domain 202 through the ligand binding site 504. The complexed molecule of the substitute antigen 508, the SHAL 502, and the MHC Class-II 200 forms an antigen presenting complex 702 for presenting the substitute antigen 508 to a T-cell.

Shown schematically in FIG. 7 is the binding of the adhesion-molecule/substitute-antigen complex at, or near, the antigen binding slot of the HLA-DR. Note that the substitute antigen is not directly bonded to the HLA-DR binding site but rather the adhesion molecule is binding at, or near, the HLA-DR binding site and the substitute antigen is bonded to the adhesion molecule via the tether. This process essentially commandeers the HLA-DR molecule and using this mechanism allows HLA-DR may to carry any antigen to which it may, or may not, previously bind. The antigen may then be in a position on the antigen presenting cell allowing it to interact with the T-cells and initiate an immune response. With a much stronger bond between the antigen presenting cell and the antigen, there is a much greater opportunity for interaction with T-cells since the on/off rate for binding may be extremely low for the synthetic complex compared to the interactions associated with the natural process.

It has been determined that SH7139, while designed to specifically bind strongly to the human protein HLA-DR, is also able to bind to the complementary MHC receptors in dogs. This may allow for research and development, safety testing, and other potential issues in a non-human host. There are numerous different variations and modifications to SH7139 that may be considered as covered by this invention. It is expected that this invention may be moved with few, if any, changes from dog to humans.

There are numerous modifications to the adhesion molecule (SH7139) that may broaden its potential for use in this invention. The length of the tether attaching the substitute antigen is expected to be a variable that may require optimization for different substitute antigens. It may also be of value to vary the chemistry of the linkers and tether. The backbone of SH7139 is three tether-linked binding sites with a fourth tether available for binding molecules for targeting. The structural elements of this adhesion molecule may be replaced with a number of different polymers. It is expected that different substitute-antigens may be preferentially bonded to adhesion molecules made from different polymers and permit alternative chemistries to be used for bonding the substitute antigen to the adhesion molecule. In addition, the solubility of the adhesion molecule may be impacted by the choice of polymer used. Polymer-substitute antigen chemical binding experimentation with the SHAL may be undertaken prior to synthesis of a new SAAMC in order to reduce labor intensive development process, i.e. the chemical synthesis of a new SHAL-substitute antigen complex.

The original strategy used in the development of SH7139 may be used to develop new adhesion molecules that are tailored for bonding to different HLA-DR epitopes and to other class-I and class-II MHC proteins. SH7139 is estimated to be able to bind to the HLA-DR of about 85-95% of the human population, and with the proper design changes another similar small-molecule may be developed that would include coverage of the missing 5 to 15% and provide overlapping options for those currently covered. SHALs optimized for other HLA-DR epitopes may also be designed and synthesized.

New adhesion molecules may also be designed to target alternative sites on the HLA-DR or other MHC proteins, including targeting regions adjacent to the natural antigen binding site rather that at the binding site proper. It is expected that steric hindrance may be a more severe problem with some substitute-antigens than with others. Moving the adhesion molecule out of the natural binding site may allow better binding of the substitute antigen to the T-cell receptor. Targeting alternative sites for the adhesion molecule to bind on the MHC proteins may also add degrees of freedom for spatially orienting the substitute antigen in the natural binding region.

Several factors may be used to alter the orientation of the antigen when bound to the MHC molecule using the adhesion molecule. This possibility allows both substitute and natural antigens to be presented to T-cells in alternative spatial orientations. This is achieved, as mentioned above, by changing how and where the adhesion molecule binds to the MHC molecules but may also be achieved by changing tether length and also the position where the tether molecule is chemically bonded onto the substitute antigen. The position of the tether binding site on the substitute antigen is a variable that is expected to affect the immune response to the vaccine.

Changes in the chemistry of SAAMC, for example attaching sugar molecules to the backbone (tether) structure, may improve the solubility of a SAAMC in buffered solution. This may also be obtained with alternative polymers in the backbone structure of the adhesion molecule or attachment of other types of molecules. It also may improve potential transport across the gut for oral administration and improve the immune response in other modes of delivery. It may also improve the immune response by altering the way the vaccine interacts with MHC and other proteins.

New SAAMCs may be prepared such that the APC binding portion is a precursor molecule that may then have attachment of different length tethers as the last steps in the adhesion molecule synthesis process. This may help avoid complete synthesis of each different adhesion molecule that is desired.

The range of molecule types for substitute antigens is essentially unlimited. Vaccines may be developed to the full range of known antigen types using this method. It may be possible to develop immunity directly to the polysaccharide coats associated with, or without, linking them to outer coat proteins. New targeting methods for cancer include directing an immune response to CD-19 and other molecules known to be expressed on tumor cells. Targeting CTLA-4 and PD-1/PD-L1 and other immune-response inhibitors may be another potential strategy utilized for cancer treatment using the presently disclosed process.

Currently monoclonal antibodies are manufactured to the targets, but the process presented here would allow the body to produce its own antibodies to these targets. This would avoid issues of developing an immune response to the foreign antibodies used in treatment. There is the possibility of completely new approaches to vaccine development, including completely synthetic molecules that mimic the shape and chemical properties of naturally occurring molecules or regions of molecules. Structural imaging and computer modeling in silico may be used to help identify the structure of key regions of targets so that substitute antigens that structurally mimic these regions may use the process described here to develop an immune response to that region. As an example, this may be the region of the flu virus that seems to exist in all flus but is very hard to develop immunity toward. This may be done by knowing the molecular topography from, for example, x-ray or protein folding analysis and using that to create a molecule that mimics it; this mimic molecule may be used as a substitute antigen.

A strong chemical bond between the adhesion-molecule and the substitute-antigen is one possible approach to development of SAAMCs. Another possibility is to use a linker system like, for example, an avidin/biotin pair to bond the adhesion molecule to the substitute antigen. Avidin, for example, may be attached to the adhesion molecule while the biotin was attached to the substitute antigen. When combined, the adhesion-molecule/substitute-antigen would form a complex with the avidin and biotin mediating the binding between the two parts. The binding pair, e.g., avidin and biotin, may be switched so that biotin was on the adhesion-molecule and avidin on the substitute-antigen. The method may refer to this process as “derivatized” adhesion molecules and substitute antigens. One of the advantages of using these kinds of binding pairs is that it may be used to make a wide variety of different vaccines by allowing the substitute antigen to be easily replaced. The derivatized adhesion molecule would be able to bind to any substitute antigen bearing the other half of the binding pair. This would allow rapid screening of different variations of adhesion-molecules selectively paired with a wide range of substitute antigens. As mentioned earlier, SH7139 is expected to only cover about 85-95% of the human population, so other adhesion molecules may be needed to cover the whole human population and new adhesion molecules may offer better performance for different groups of patients. Being able to combine a derivatized adhesion molecule with its counter-derivatized substitute antigen would reduce the amount of time and chemical synthesis required when developing a new vaccine. Effectively, any adhesion molecule may be bound to any substitute antigen using the linker system in place of a chemical reaction like the carbodiimide chemistry used to make SH7139. The derivatized vaccine precursors may be stored separately and combined as needed. Other bonding pairs, including various selective chemically reactive pairs, may be used to accomplish this.

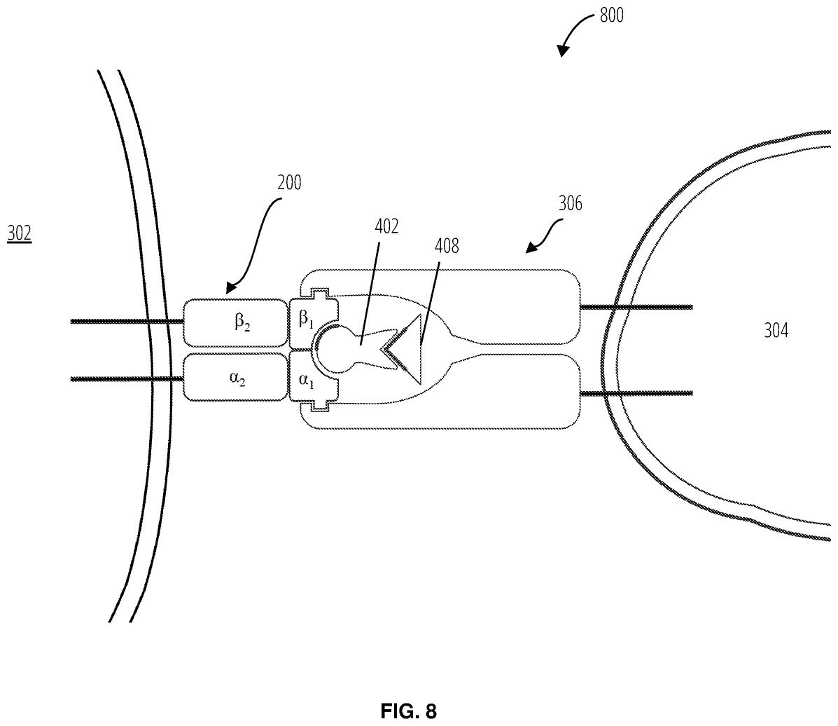

Referencing FIG. 8, a schematic diagram 800 shows the presentation of the substitute antigen 408 to a T-cell 304 through the coupling of the MHC Class-II 200 and a T-cell receptor 306. During the engagement between the T-cell receptor 306 and the MHC Class-II 200, the substitute antigen 408 is released to the T-cell 304 stimulating humoral immunity and the production of antibodies for the substitute antigen 408.

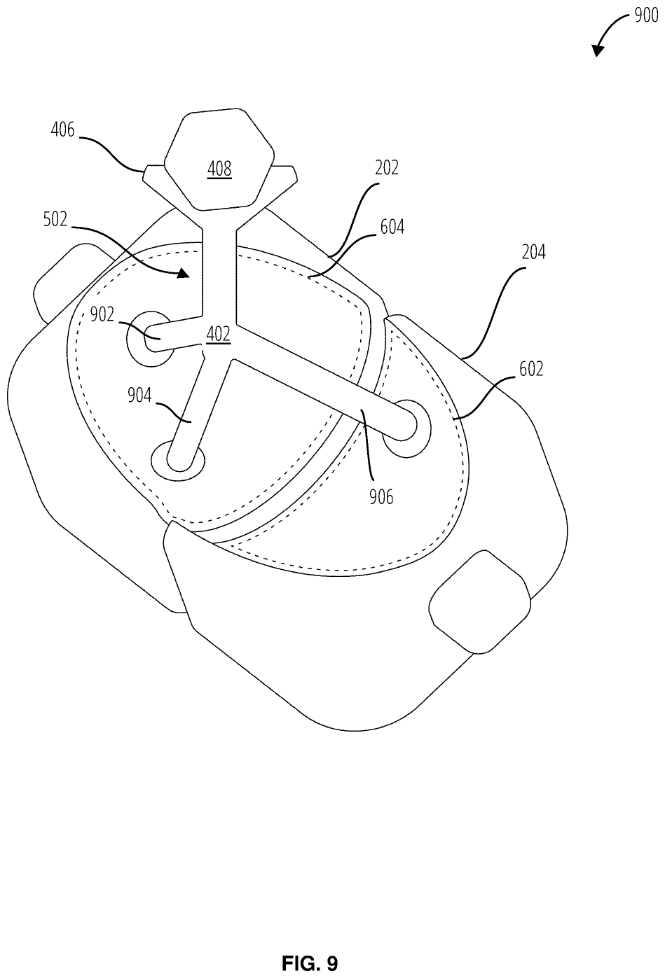

FIG. 9 illustrates a diagram 900 showing a configuration of the SHAL 402 comprising three ligands (a first ligand 902, a second ligand 906, and a third ligand 904). The diagram 900 shows the first ligand 902 and the second ligand 906 bound to the β₁ domain binding region 604 of the β₁ domain 202. The third ligand 904 is bound adjacent to the β₁ domain binding region 604 in the α₁ domain binding region 602 of the α₁ domain 204. The adjacent binding configuration may accommodate steric interference due to a large molecule structure and/or molecular forces associated with the substitute antigen 408.

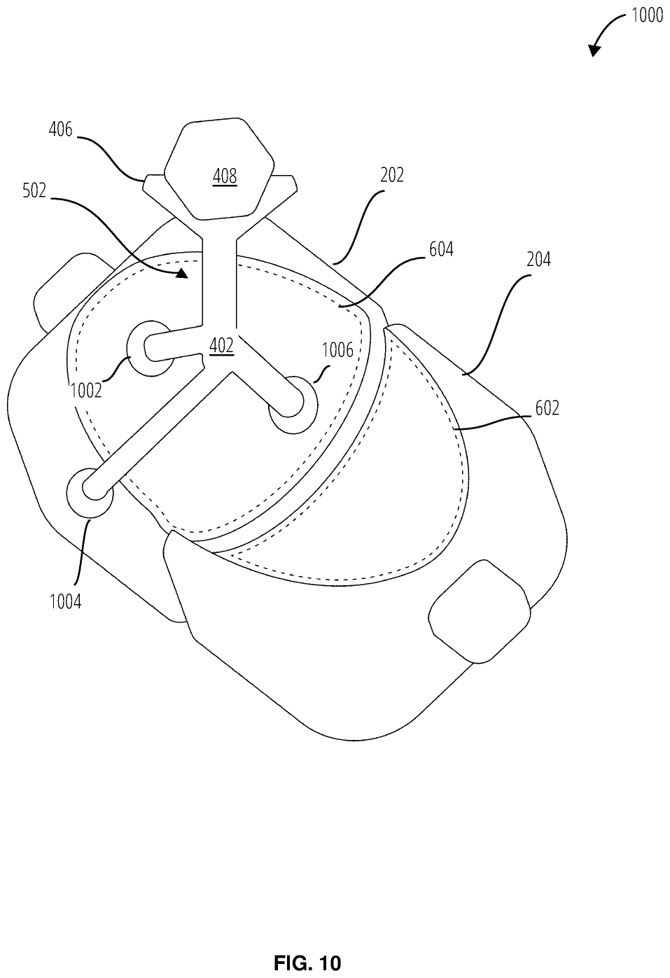

FIG. 10 illustrates a diagram 1000 showing a configuration of the SHAL 402 comprising three ligands (a first ligand 1002, a second ligand 1004, and a third ligand 1006). The diagram 900 shows the first ligand 1002 and the third ligand 1006 bound to the β₁ domain binding region 604 of the β₁ domain 202. The second ligand 1004 is bound adjacent to the β₁ domain binding region 604 but on the β₁ domain 202 outside of the β₁ domain binding region 604. The adjacent binding configuration may accommodate steric interference due to a large molecule structure and/or molecular forces associated with the substitute antigen 408.

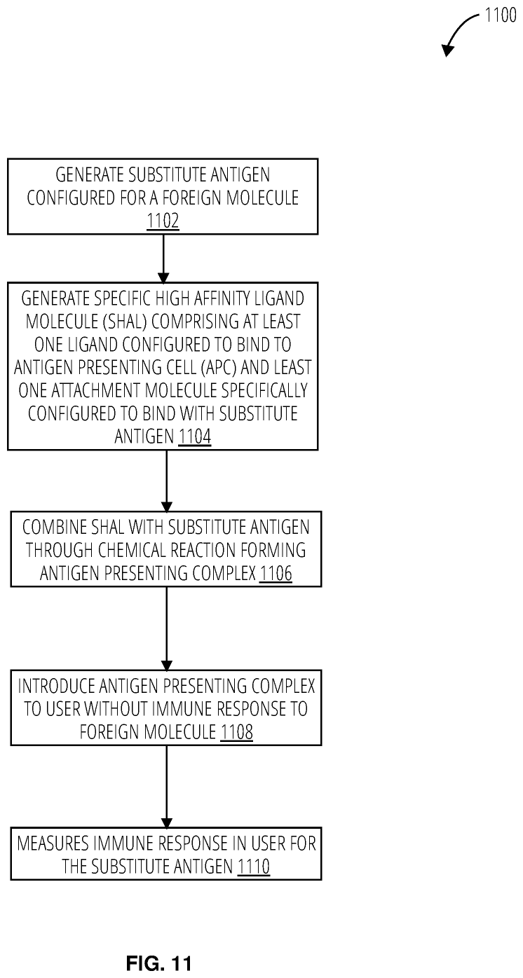

Referencing FIG. 11, in block 1102, the method 1100 generates a substitute antigen configured for a foreign molecule (e.g., a pathogen, toxicant, or etc.). In block 1104, the method 1100 generate a synthetic high affinity ligand molecule (SHAL) comprising at least one ligand configured to bind to an antigen presenting cell (APC) and at least one attachment molecule specifically configured to bind with the substitute antigen. In block 1106, the method 1100 combines the SHAL with the substitute antigen through a chemical reaction forming an antigen presenting complex. In block 1108, the method 1100 introduces the antigen presenting complex to a user without an immune response to the pathogen or toxicant. In block 1110, the method 1100 measures the immune response in the user for the substitute antigen. The aforementioned step may function as a feedback mechanism in which the method may identify if the substitute antigen generates a humoral response and may further involve introducing a new antigen presenting complex comprising a variant substitute antigen, in response to the corresponding antibody being below a minimum threshold.

In order to have the adhesion molecule bond to a different region of the HLA-DR, a new SHAL adhesion molecule may be developed using the methodology in the SHAL patent. The SH7139 molecule was designed using in silico computer modeling techniques combined with experiments. A docking program was used to screen hundreds of thousands of potential molecules that might bind to the sites of interest. These were down selected to a few dozen which were studied for their binding strength and location specificity. These experiments were used to further down select to the three targeting molecules that were linked together with molecular tethers. Computer simulations of the targeting molecules bound to the HLA-DR10 molecule were used to determine the lengths of the tethers and the final SH7139 molecule also studied with computer studies to determine if it would bind. These were in turn followed by extensive experimental studies of the binding of SH7139 to HLA-DR10.

In order to change the portion of HLA-DR to which the adhesion molecule may attach, the same methodology used to develop SH7139 may be followed in order to have the adhesion molecule to bind near, rather than at, the native antigen binding site. This includes docking hundreds of thousands of potential targeting molecules, a down-selection of molecules for experimental testing, further down-selection to molecules that may be tethered together to form a new SHAL adhesion molecule that binds to a region near the native antigen binding site, leaving the binding site more available for the substitute antigen to occupy the native binding site.

### Importance of 3D Computer Models of Targeted Disease Molecules

The knowledge gained from computer-based models of proteins associated with foreign molecules—may be instrumental in determining the best AA sequences for targeting by showing where those are located in three-dimensions (3D) on the protein. Ideally, they are on the exterior regions of the protein and preferably stay exposed as the protein undergoes modifications in the body. Coverage of those exterior regions by polysaccharides is also important to know when targeting glycoproteins. The amino acid sequences of each region of the protein are often well known. Amino acid sequences that constitute all of, or portions, of each region may be custom synthesized commercially. Large libraries of such sequences are routinely assembled as needed.

Other kinds of studies of importance to selecting the best substitute antigen are those that identify the epitopes for which antibodies have been able to be made to regions of the protein of interest. These studies are often done in [non-human] animals and are ideally suited for this method. Peptides with the same AA sequence as the epitope that is targeted by antibodies, may be manufactured and attached to the adhesion molecule for development of an adaptive immune response to the protein.

A general strategy for utilization of this invention's technology may be to develop a set of peptides of length (i.e. 6 to 9 AAs) as the substitute antigens to be bound to the HLA-DR via the attachment molecule. In some configurations, the invention may utilize peptide with lengths greater than 9 AAs but with a functional portion (i.e., epitope region) being between 6-9 AAs. The greater length may be provided to facilitate binding to the adhesion molecule or assist in the presentation of the functional portion to T-cells. Typically, HLA-DR binds peptides six to eight amino acids in length. Peptide sequences for testing are selected from those regions of greatest interest that are identified using the 3D structure of the protein. Sequences known to be exposed on the surface of the protein, and other regions where an antibody would have a better chance of access due to limited steric effects, would be chosen preferentially. The sets of peptide sequences would consist of overlapping amino acid sequences.

The peptides serving as substitute-antigens are chemically bonded to the attachment molecule using any one of a large number of possible chemistries. In addition to commercial peptide sources, or laboratory peptide synthesis, approaches to selection of antigens for the method may include:
completely chemically synthesized molecules of any composition—including those designed by computer modeling to mimic the target region⋅molecules purified from biological sources
molecules produced using recombinant-DNA methods
as well as chemical modifications to any of the above.

Substitute antigens may often be chemically linked to the adhesion molecule at several locations in the substitute-antigen peptide. These include both the amide and carboxyl terminal ends, as well as various chemically active groups on the side chains of AAs. Non-proteinogenic AAs similar to the AAs in the identified substitute-antigen having usable chemical side-chains with desirable linkage chemistries, maybe substituted for AAs in the identified substitute-antigen.

The variety of possible binding sites on the substitute-antigen peptide provides a mechanism for changing the orientation and positioning of the peptide for presentation to the T-cell.

### Example Application #1: Virus: Universal Flu Vaccine

A key focus of influenza research today is the development of a universal flu vaccine, or a vaccine that provides robust, long-lasting protection against multiple subtypes of flu, rather than a select few. Such a vaccine would eliminate the need to update and administer the seasonal flu vaccine each year and may provide protection against newly emerging flu strains, potentially including those that may cause a flu pandemic.

The general approach here is to target the invariant part of the ectodomain of a membrane bound flu virus protein. Flu viruses are classified by two proteins on the outer surface of the virus: hemagglutinin (H) and neuraminidase (N). There are 18 different H subtypes and 11 different N subtypes, and viruses may be further broken down into different strains within those subtypes. Subtypes of the virus are identified by the H and N subtypes, such as H1N1. The H protein (also called HA) which enables the flu virus to enter a human cell, is made up of a head and a stem. Seasonal flu vaccines fight infection by inducing antibodies that target the HA head. This region varies season to season, which is why flu vaccines must be updated each year. However, scientists have discovered the stem typically remains unchanged, making it an ideal target for antibodies induced by a universal flu vaccine.

The HA molecule has a large ectodomain of ≈500 AA. A posttranslational cleavage by host-derived enzymes generates 2 polypeptides that remain linked by a disulfide bond. The larger N-terminal fragment (HA1, 320-330 AA) forms a membrane-distal globular domain that contains the receptor-binding site and most determinants recognized by virus-neutralizing antibodies related to current vaccines for the variant parts of the virus. The smaller C terminal portion (HA2, ≈180 AA, excluding transmembrane and cytoplasmic domain) forms a stem like structure that anchors the globular domain [HA head] to the cellular or viral membrane. Although the degree of sequence diversity between subtypes is great, particularly in the HA1 polypeptides (34%-59% homology between subtypes), more conserved regions are found in HA2 (51%-80% homology between subtypes). The most notable region of conservation is the sequence around the cleavage site, particularly the HA2 N terminal 11 AAs, termed fusion peptide, which is conserved among all influenza A subtypes and differs only by 2 conservative AA replacements in influenza B virus. The complete fusion peptide has 19 AAs, with the 11 being highly conserved, especially among all A type viruses. Part of this region is exposed as a surface loop in the HA precursor molecule (HA0). However, when HA0 is cleaved into HA1/HA2, the newly generated terminals separate, and the hydrophobic fusion peptide becomes tucked into a cavity of the stem. As most HA subtypes are cleaved by extracellular enzymes, this surface loop may be accessible to antibodies, at least temporarily, on HA0 expressed in the plasma membrane of infected host cells.

This method may be ideal for use in the strategy of targeting the HA “stem” portion, specifically the fusion peptide at the joint-region between the head and stem. The 11 AA portion of the fusion peptide has the sequence G-L-F-G-A-I-A-G-F-I-E (Seq ID No: 01). The chemical bond between the fusion peptide and the SHAL adhesion molecule may be made at numerous points on a substitute antigen derived from this sequence. A substitute antigen would consist of a sequence of 6 to 9 AAs selected from this sequence of 11 AAs. Changing the attachment point may affect the orientation of the peptide for presentation to T-cells and this may in turn impact the effectiveness in eliciting the desired immune response. Both the amide group at the N-terminus and the carboxyl group at the C-terminus are good sites for bonding. In addition, the side chains of the amino acids are possible locations for forming bonds; in particular, the benzyl group of Phenylalanine (positions 3 and 10). Vaccine development involves selecting several sequences of six to nine continuous AAs that make up the fusion peptide and having them synthesized commercially with changing the portion of the sequence selected possibly being part of the development. It is also possible to replace amino acids without useful functional groups with those having more useful functional groups, for example, glycine in position-4 may be replaced with AAs having more easily reacted side-chains like Serine or Cysteine in this case. It is also possible to use non-proteinogenic AAs that may be considered for these purposes, AAs derivatized for easy reactivity to the SHAL. Variations in the size of the AA filling a position, in particular the length of a given side chain, may also be used to effectively change the tether length between the substitute antigen and the adhesion molecule.

### Example Application #2: A Second Approach to a Universal Flu Vaccine

The general approach is the same and starts by identifying an invariant region of a viral protein's ectodomain. Another approach to a universal flu vaccine has been based on targeting the ectodomain domain of the transmembrane viral M2-protein (M2e) [Nature Medicine 5 (1999) 1119]. This too is an excellent candidate for this method. An advantage of using M2e as the antigen is the conservation of its sequence, with few changes documented since the first influenza virus was isolated in 1933, despite numerous epidemics and several pandemics. The M2-protein is present in only small amounts on the virion but is expressed in large numbers on virus-infected cells.

M2 has a small, nonglycosylated ectodomain (i.e. M2e) of 23 amino acids (aa), not counting the post translationally removed N-terminal Met. This region has shown only limited variation among human influenza A viruses. This remarkable degree of structural conservation of M2e is attributable mainly to its genetic relation with matrix protein 1 (M1), the most conserved protein of influenza A viruses with which it shares coding sequences. Thus, AA residues 1-9 of M2e and M1 are encoded by the same nucleotides in the same reading frame and aa 10-23 of M2e and 239-252 of M1 in a different reading frame.

The adhesion molecule method targets the ectodomain of the influenza protein M2, known as M2e. The 23 AA portion of the peptide has the sequence M-S-L-L-T-E-V-T-P-I-R-N-E-W-G-C-R-C-N-D-S-S (Seq ID No: 02). Crystallographic x-ray studies with a Fab of a protective M2e-specific monoclonal antibody have been used to identify a region M2e that is susceptible to attack by antibodies. In this complex, M2e adopts a U shape with M2-Trp15 positioned in the center. The structure reveals a critical role for M2-Glu6, -Pro10, -Ile11, and -Trp15 in monoclonal antibody binding.

The chemical bond between M2e and the SHAL adhesion molecule may be made at numerous points on a substitute antigen derived from this sequence keeping in mind the important role of the nine AA sequence from -Pro10 through -Trp5. A substitute antigen would consist of a sequence of 6 to 9 AAs selected from this sequence. Changing the attachment point may affect the orientation of the peptide for presentation to T-cells and this may in turn impact the effectiveness in eliciting the desired immune response. Both the amide group at the N-terminus and the carboxyl group at the C-terminus of the substitute antigen are good sites for bonding to the adhesion molecule. In addition, the side chains of the amino acids are possible locations for forming bonds. There are many more options for chemical bonding even to this short sequence that include the groups on the side chains of -Glu6, The8, -Pro9, -Arg11, -Asp12, -Glu13, and -Trp14. Vaccine development involves selecting a sequence of six to nine continuous AAs that make up the fusion peptide and having them synthesized commercially. Changing the portion of the sequence selected may also be part of the development. It is also possible to replace amino acids without useful functional groups with those having more useful functional groups, for example, -val7 may be replaced with AAs having more easily reacted side-chains like Serine or Cysteine in this case. It is also possible to use non-proteinogenic AAs that may be considered for these purposes, specifically AAs derivatized for easy reactivity to the SHAL. Variations in the size of the AA filling a particular position in the sequence, especially the length of a given AA side chain, may also be used to effectively change the tethering length between the substitute antigen and the adhesion molecule, i.e., part of the tether would now be part of the substitute antigen.

### Example Application #3: Capsular Polysaccharide Vaccine; Staphylococcus aureus

Capsular polysaccharides (PSs) are highly polar, hydrophilic cell surface polymers consisting of oligosaccharide repeating units. These molecules are the main antigens involved in the protective immunity to encapsulated bacteria. Capsular PSs interfere with bacterial interactions with phagocytes by blocking opsonization. Opsonization is the coating of the organisms by specific antibodies and complement, which enables host phagocytes to ingest and destroy invading bacteria. Antibodies bound to capsular PSs may act as bacterial-cell-to-phagocytic-cell ligands or as complement activators.

The response to a capsular PS is T-cell-independent, meaning that B lymphocytes proliferate and produce antibody without the help of T cells. Conjugation is a method that couples a PS to a protein carrier, which changes the capsular PS from a T-cell-independent antigen to a T-cell-dependent antigen. The immune response elicited by this protein antigen uses helper T cells and thus is T-cell-dependent. Helper T cells enable a more rapid and enhanced immune response to occur on re-exposure to an antigen. In this manner, a conjugate vaccine induces immunologic memory and provides long-term protective immunity.

The adhesion molecule method described here may appear to be similar to a conjugate vaccine, however it is very different. The conjugate vaccine still relies on the phagocytosis of the protein-PS conjugate and subsequent processing by a consuming cell to generate antigens. In contrast to this, the adhesion molecule method circumvents the cell consumption step and directly presents the PS sequence to T-cells at the APC surface. The PS is not attached to just any protein with the adhesion molecule, it is directly attached to HLA-DR or another relevant protein on the surface of the APC. The PS chains evidently may produce their own immune response, but it is not very robust immune response.

Serotype 5 and 8 capsular polysaccharides predominate among clinical isolates of Staphylococcus aureus. The results of experiments in animal models of infection have revealed that staphylococcal capsules are important in the pathogenesis of S. aureus infections. The capsule enhances staphylococcal virulence by impeding phagocytosis, resulting in bacterial persistence in the bloodstream of infected hosts. Although the capsule has been shown to modulate S. aureus adherence to endothelial surfaces in vitro, animal studies suggest that it also promotes bacterial colonization and persistence on mucosal surfaces. S. aureus capsular antigens are surface associated, limited in antigenic specificity, and highly conserved among clinical isolates. With the emergence of vancomycin-resistant S. aureus in the United States in 2002, new strategies are needed to combat staphylococcal infections. Purified serotype 5 and 8 capsular polysaccharides offer promise as target antigens for a vaccine to prevent staphylococcal infections, although the inclusion of other antigens is likely to be essential in the development of an effective S. aureus vaccine. The genetics and mechanisms of capsule biosynthesis are complex, and much work remains to enhance the understanding of capsule biosynthesis and its regulation.

Type 5 and 8 capsular polysaccharides (CP5 and CP8, respectively) purified from the prototype strains Reynolds and Becker, respectively, are structurally very similar to each other and to the capsule made by strain T. Type 5 has the structure (→4)-3-O-Ac-β-D-ManNAcA-(1→4)-α-L-FucNAc-(1→3)-β-D-FucNAc-(1→)ₙ, and type 8 has the structure (→3)-4-O-Ac-β-D-ManNAcA-(1→3)-α-L-FucNAc-(1→3)-β-D-FucNAc-(1→)ₙ. Type 5 and 8 polysaccharides differ only in the linkages between the sugars and in the sites of O-acetylation of the mannosaminuronic acid residues, yet they are serologically distinct. The structure of CP4 was never elucidated, although the prototype serotype 4 strain 7007 has been shown to react with antibodies to CP5. CP4 is most likely a polysaccharide with a trisaccharide structure identical to that of CP5 but differing in the presence, absence, or location of the O-acetyl moieties on the N-acetylmannosaminuronic acid residues.

The chemical bond between CP5 or CP8 and the SHAL adhesion molecule may be made at numerous points on a substitute antigen derived from these sequences of sugars. In the case of sugar molecules, there are a large number of chemically-similar sites. The use of sugars that have been derivatized in order to make use of more selective chemistries than those used for bonding polypeptides may be employed. The location of the derivatization may be selected in order to change the orientation of the polysaccharide in the HLA-DR binding site. Changing the attachment point of the adhesion molecule to the polysaccharide may affect the orientation of the peptide for presentation to T-cells and this may in turn impact the effectiveness in eliciting the desired immune response.

### Example Application #4: Tuberculosis Vaccine

Tuberculosis (TB) is an infectious disease usually caused by the bacterium Mycobacterium tuberculosis (MTB). Tuberculosis generally affects the lungs, but may also affect other parts of the body. Most infections do not have symptoms, in which case it is known as latent tuberculosis. About 10% of latent infections progress to active disease which, if left untreated, kills about half of those infected. The classic symptoms of active TB are a chronic cough with blood-containing sputum, fever, night sweats, and weight loss. The historical term “consumption” came about due to the weight loss. Infection of other organs may cause a wide range of symptoms.

Tuberculosis is spread through the air when people who have active TB in their lungs cough, spit, speak, or sneeze. People with latent TB do not spread the disease. Active infection occurs more often in people with HIV/AIDS and in those who smoke. Diagnosis of active TB is based on chest X-rays, as well as microscopic examination and culture of body fluids. Diagnosis of latent TB relies on the tuberculin skin test (TST) or blood tests.

Prevention of TB involves screening those at high risk, early detection and treatment of cases, and vaccination with the bacillus Calmette-Guérin (BCG) vaccine. Those at high risk include household, workplace, and social contacts of people with active TB. Treatment requires the use of multiple antibiotics over a long period of time. Antibiotic resistance is a growing problem with increasing rates of multiple drug-resistant tuberculosis (MDR-TB) and extensively drug-resistant tuberculosis (XDR-TB).

Presently, one-quarter of the world's population is thought to be infected with TB. New infections occur in about 1% of the population each year. In 2016, there were more than 10 million cases of active TB which resulted in 1.3 million deaths. This makes it the number one cause of death from an infectious disease. More than 95% of deaths occurred in developing countries, and more than 50% in India, China, Indonesia, Pakistan, and the Philippines. The number of new cases each year has decreased since 2000. About 80% of people in many Asian and African countries test positive while 5-10% of people in the United States population test positive by the tuberculin test. Tuberculosis has been present in humans since ancient times.

The only available vaccine as of 2011 is Bacillus Calmette-Guérin (BCG). In children it decreases the risk of getting the infection by 20% and the risk of infection turning into active disease by nearly 60%.

It is the most widely used vaccine worldwide, with more than 90% of all children being vaccinated. The immunity it induces decreases after about ten years. As tuberculosis is uncommon in most of Canada, the United Kingdom, and the United States, BCG is administered to only those people at high risk. Part of the reasoning against the use of the vaccine is that it makes the tuberculin skin test falsely positive, reducing the test's usefulness as a screening tool.

TB remains the first cause of death from a single infectious agent despite the availability of the BCG vaccine. TB still causes more than a million deaths per year in spite of a 47% drop in TB mortality rate since 1990. Although BCG is protective against disseminated disease in young children, it has variable efficacy against pulmonary TB, particularly in adults. A more consistently effective vaccine than BCG in both adolescents and adults is needed to achieve the ‘End TB strategy’ set by the World Health Organization. BCG is an attenuated strain of Mycobacterium bovis. The loss of virulence of this strain is caused by the deletion of the RD-1 locus that encodes nine genes including a 10-kDa cultured filtered protein (CFP-10) and a 6-kDa early secreted-antigen (ESTAT-6). Both are proteins secreted by the Snm secretion system and are considered essential virulence factors contributing to M.tb pathogenesis, suggesting that they may be good vaccine targets. ESAT-6 was shown to block TLR2 at the surface of the macrophage and CFP-10/ESAT-6 complex was shown to downregulate reactive oxygen species (ROS) production. In addition, CFP-10/ESAT-6 complex was shown to dissociate under acidic condition allowing ESTAT-6 to destabilize and lyse liposomes. The lack of expression of CFP-10 and ESAT-6 by Mycobacterium bovis in the BCG vaccine prevents the bacteria to counteract its destruction by the host cell, allowing it to kill M.tb efficiently. The destruction of the pathogen makes M.tb antigens available allowing the subsequent activation of CD4 and CD8 cells via antigen presenting cells and the production of Interferon-γ (IFN-γ), a key cytokine in the immune response against M.tb.

ESAT-6, an abundantly secreted protein of Myobacterium tuberculosis (M. tuberculosis) is an important virulence factor, inactivation of which leads to reduced virulence of M. tuberculosis. ESAT-6 alone, or in complex with its chaperone CFP-10 (ESAT-6:CFP-10), is known to modulate host immune responses; however, the detailed mechanisms are not well understood. The structure of ESAT-6 or ESAT-6:CFP-10 complex does not suggest the presence of enzymatic or DNA-binding activities. Therefore, it has been hypothesized that the crucial role played by ESAT-6 in the virulence of mycobacteria may be due to its interaction with some host cellular factors. Using a yeast two-hybrid screening, it has been identified that ESAT-6 interacts with the host protein beta-2-microglobulin (β₂M), which was further confirmed by other assays, like GST pull down, co-immunoprecipitation and surface plasmon resonance. The C-terminal six amino acid residues (90-95) of ESAT-6 were found to be essential for this interaction. ESAT-6, in complex with CFP-10, also interacts with β₂M. It has been found that ESAT-6/ESAT-6: CFP-10 may enter into the endoplasmic reticulum where it sequesters β₂M to inhibit cell surface expression of MHC-I-β₂M complexes, resulting in downregulation of class I-mediated antigen presentation. Interestingly, the ESAT-6: β₂M complex may be detected in pleural biopsies of individuals suffering from pleural tuberculosis. Data point to a novel mechanism by which M. tuberculosis may undermine the host adaptive immune responses to establish a successful infection. Identification of such novel interactions may help in designing small molecule inhibitors as well as an effective vaccine design against tuberculosis. Using the yeast two hybrid assay, it was observed that the deletion of the last 6 amino acids (valine, threonine, glycine, methionine, phenylalanine and alanine) from the C-terminal end of ESAT-6 was sufficient to prevent the interaction of ESAT-6 and β₂M.

The adhesion molecule method is ideal for use in the strategy of targeting ESAT-6, specifically the six AA sequence at the C-terminus end. The six AAs portion has the sequence (valine, threonine, glycine, methionine, phenylalanine and alanine: V-T-G-M-F-A (Seq ID No: 03)). The chemical bond between an ESAT-6 peptide and the SHAL adhesion molecule may be made at numerous points on a substitute antigen derived from this sequence. A substitute antigen would consist of these six AAs but might also include additional amino acids that are adjacent to the N-terminus of these six. Changing the attachment point may affect the orientation of the peptide for presentation to T-cells and this may in turn impact the effectiveness in eliciting the desired immune response. Both the amide group at the N-terminus and the carboxyl group at the C-terminus of the substitute-antigen peptide are good sites for bonding. In addition, the side chains of the amino acids are possible locations for forming bonds; including the benzyl group on phenylalanine, the sulfide group on methionine, and the hydroxyl group on threonine. These peptides are to be synthesized commercially. It is also possible to replace amino acids without useful functional groups with those having more useful functional groups, for example, glycine may be replaced with AAs having more easily reacted side-chains like Serine or Cysteine in this case. It is also possible to use non-proteinogenic AAs that may be considered for these purposes, AAs derivatized for easy reactivity to the SHAL. Variations in the size of the AA filling a position, in particular the length of a given side chain, may also be used to effectively change the tether length between the substitute antigen and the adhesion molecule.

Efficacy is tested by measuring changes in Antigen 85B-specific immune responses in eight immunological assays (blood lymphocyte proliferation, antibody responses by ELISA, interferon-gamma producing CD4+ and CD8+ T cells ex vivo, central memory CD4+ and CD8+ T cells, interferon-gamma ELISPOT responses, and the capacity of T cells to activate macrophages to inhibit mycobacterial intracellular multiplication).

Details on previous formation and utilization of the SHAL can be found in U.S. application Ser. No. 11/055,181, filed Feb. 9, 2005, now U.S. Pat. No. 7,662,785, which has been incorporated by reference below.

In certain embodiments this invention provides novel polydentate selective high affinity ligands (SHALs) that may function to specifically bind particular target molecules in a manner analogous to antibody binding. Methods for the design and generation of SHALs as affinity reagents for basic or applied research or for diagnosis and treatment of infectious and/or malignant diseases and their administration to patients with infectious and/or malignant diseases are described.

The SHALs typically comprise two or more ligands (binding moieties) that each bind different regions on the intended target attached to each other directly or through a linker. Where the SHAL is directed to a marker on a cancer cell, the SHAL associates in greater density (abundance) or accessibility on the target cell as compared to normal cells. The SHAL thus provides selectivity appropriate for diagnosis or treatment of the target cells. Different SHALs may be readily generated for different malignant cells and malignant diseases. The SHAL represents a core building block (e.g., a targeting moiety) that may be incorporated into larger e.g., chimeric molecules to affect specific delivery of an effector to the target.

Thus, in one embodiment, this invention provides a method of making a selective high-affinity polydentate ligand (SHAL) that specifically binds a target molecule. In certain embodiments, the method typically involves screening a first ligand library to identify a first ligand that binds to the target molecule; screening a second ligand library to identify a second ligand that binds to the target molecule where the second ligand is different than the first ligand; linking the first ligand to the second ligand to form a SHAL; and screening the SHAL for the ability to specifically bind to the target molecule. In certain embodiments the target molecule is a protein. In certain embodiments the target molecule is a cancer marker (e.g., Lym-1 epitope, Muc-1, C-myc, p53, Ki67, Her2, Her4, BRCA1, BRCA2, Lewis Y, CA 15-3, G250, HLA-DR cell surface antigen, CEA, CD20, CD22, integrin, cea, 16, EGFr, AR, PSA, and other growth factor receptors, etc.). The method may optionally further involve screening the SHAL to identify a SHAL that binds to the target with an avidity and/or specificity higher than either ligand comprising the SHAL. The first ligand library and the second ligand library may be the same library or may be different libraries. In certain embodiments the first and/or second ligand library is a library of small organic molecules. In certain embodiments screening the first ligand library and/or screening the second ligand library comprises virtual in silico screening. The virtual in silico screening may comprise screening a compound database (e.g., MDL® Available Chemicals Directory, ChemSpider, GNU-Darwin) using one or more algorithms as utilized in the DOCK program. The virtual in silico screening may comprise screening a compound database using the DOCK program. The virtual in silico screening may involve screening for a first ligand and/or multiple ligands that bind a pocket on a protein. In certain embodiments the pocket is identified using an algorithm utilized by the SPHGEN program. In certain embodiments the pocket is identified using the SPHGEN program. The virtual in silico screening may involve screening for a second or third ligand that binds different regions of the target than the ligands identified when screening the first ligand library.

In certain embodiments screening a first ligand library and/or screening a second ligand library additionally comprises screening one or more ligands identified in the virtual in silico screening in a physical assay for the ability to bind to the target. Suitable physical assays include, but are not limited to a BIAcore assay, saturation transfer difference nuclear magnetic resonance spectroscopy, and transfer NOE (trNOE) nuclear magnetic resonance spectroscopy, ELISA, competitive assay, tissue binding assay, a live cell binding assay, a cellular extract assay, and the like.

Linking of the ligands may involve directly linking two or more ligands or linking two or more ligands with a linker (e.g., a PEG type linker, a peptide linker, an avidin/biotin linker, a straight chain carbon linker, a heterocyclic linker, a branched carbon linker, a dendrimer, a nucleic acid linker, a thiol linker, an ester linker, a linker comprising an amine, a linker comprising a carboxyl, etc.). The linking may optionally comprise linking two or more ligands with linkers of different lengths to produce a library of SHALs having different length linkers; and, optionally, screening the library of SHALs having different length linkers to identify members of the library that have the highest avidity and/or specificity for the target. In certain embodiments the method further involves comprising screening the SHAL(s) to identify a SHAL that binds to the target with an avidity and/or specificity higher than either ligand comprising the SHAL. The screening of individual ligands and/or bivalent or polyvalent SHAL(s) may be by any of a variety of methods including, but not limited to a BIAcore assay, saturation transfer difference nuclear magnetic resonance spectroscopy, and transfer NOE (trNOE) nuclear magnetic resonance spectroscopy, ELISA, competitive assay, tissue binding assay, live cell binding assay, a cellular extract assay, and the like. In certain embodiments the target molecule is a protein and/or a cancer marker (e.g., a Lym1 epitope, Muc-1, C-myc, p53, Ki67, Her2, Her4, BRCA1, BRCA2, Lewis Y, CA 15-3, G250, HLA-DR cell surface antigen, etc.).

Also provided is a method of synthesizing an inhibitor for an enzyme or other binding protein or receptor. In certain embodiments the method typically involves identifying a first pocket (or bump) and a second or third pocket (or bump) in the enzyme or other binding protein or receptor where the first, second and third pockets flank opposite sides of the active site or binding site of the enzyme or other binding protein or receptor; screening a first ligand library to identify a first ligand that binds to the first pocket (or bump); screening a second ligand library to identify a second ligand that binds to the second pocket (or bump); screening a third ligand library to identify a third ligand that binds to a third pocket or bump; linking the first ligand to the second and third ligands to form a polydentate selective high affinity ligand (SHAL); and screening the SHAL for the ability to specifically bind to and inhibit the enzyme or other binding protein. In certain embodiments the pockets or “bumps” need not be located on opposite sides of the active site or binding site of the enzyme or binding protein or receptor, but are simply located so that binding of the SHAL blocks binding of the native cognate ligand to that site. In certain embodiments the target molecule comprises a molecule selected from the group consisting of a protein, an enzyme, a nucleic acid, a nucleic acid binding protein, and a carbohydrate. In certain embodiments the method further involves screening the SHAL to identify a SHAL that binds to the target with an avidity and/or specificity higher than either ligand comprising the SHAL. The first, second and third ligand libraries may be the same library or may be different libraries. In certain embodiments the first and/or second and/or third ligand library is a library of small organic molecules. In certain embodiments screening the first, second and/or third ligand library comprises virtual in silico screening. The virtual in silico screening may comprise screening a compound database (e.g., MDL® Available Chemicals Directory) using one or more algorithms as utilized in the DOCK program. The virtual in silico screening may comprise screening a compound database using the DOCK program. The virtual in silico screening may involve screening for a first, second and/or third ligand that binds a pocket on a protein. In certain embodiments the pocket is identified using an algorithm utilized by the SPHGEN program. In certain embodiments the pocket is identified using the SPHGEN program. The virtual in silico screening may involve screening for a second ligand that binds different region of the target than the ligands identified when screening the first ligand library.

In certain embodiments screening a first, second or third ligand library and/or screening a second ligand library additionally comprises screening one or more ligands identified in the virtual in silico screening in a physical assay for the ability to bind to the target. Suitable physical assays include, but are not limited to a BIAcore assay, saturation transfer difference nuclear magnetic resonance spectroscopy, and transfer NOE (trNOE) nuclear magnetic resonance spectroscopy, ELISA, competitive assay, tissue binding assay, a live cell binding assay, a cellular extract assay, and the like.

Linking of the ligands may involve directly linking two or more ligands or linking two or more ligands with a linker (e.g., a PEG type linker, a peptide linker, an avidin/biotin linker, a straight chain carbon linker, a heterocyclic linker, a branched carbon linker, a dendrimer, a nucleic acid linker, a thiol linker, an ester linker, a linker comprising an amine, a linker comprising a carboxyl, etc.). The linking may optionally comprise linking the ligands with linkers of different lengths to produce a library of SHALs having different length linkers; and, optionally, screening the library of SHALs having different length linkers to identify members of the library that have the highest avidity and/or specificity for the target.

This invention also provides a polydentate selective high affinity ligand (SHAL) that specifically binds to a desired target (e.g., a cancer cell). Where the target is a cancer cell, the SHAL typically comprises a first ligand that binds to a first site on a marker for the cancer cell linked (directly or through a linker) to a second or third ligand that binds to a second or third site on same marker or on a different marker for the cancer cell where the first site, the second site and the third site are different sites (e.g., all three ligands are capable of simultaneously binding to the target(s)). In certain embodiments the first site, second site and/or the third site is a pocket (or “bump”) on the marker(s). Suitable markers include, but are not limited to a Lym-1 epitope, Muc-1, C-myc, p53, Ki67, Her2, Her4, BRCA1, BRCA2, Lewis Y, CA 15-3, G250, HLA-DR cell surface antigen, CEA, CD20, CD22, integrin, CEA, 16, EGFr, AR, PSA, other growth factor receptors, and the like.

In certain preferred embodiments, the marker is an HLA-DR cell surface antigen. In certain embodiments the three ligands bind sites within an epitope recognized by the Lym-1 or other HLA-DR specific antibodies. In certain embodiments the three ligands are each small organic molecules. In certain embodiments, the first ligand is a ligand selected from Table 1, and the second and third ligand, when present, are independently selected from the ligands in Tables 1, 5, 6, 7, or 8. In certain embodiments the SHAL comprises a first, second and/or third ligand selected from Tables 2, 3, or 4. In certain embodiments the SHAL comprises a first, second and/or third ligand selected from Table 4. The three ligands may be joined directly together or the first ligand may be attached to the second and/or third ligand by a linker (e.g., a PEG type linker, a peptide linker, an avidin/biotin linker, a straight chain carbon linker, a heterocyclic linker, a branched carbon linker, a dendrimer, a nucleic acid linker, a thiol linker, an ester linker, a linker comprising an amine, a linker comprising a carboxyl, etc.). In certain embodiments the SHAL has an avidity for the marker greater than about 10⁻⁶ M while the individual ligands comprising the SHAL each have a binding affinity for the marker less than about 10⁻⁶ M. In certain embodiments the SHAL has a formula as shown herein and, in the Figures, or is an analogue thereof.

This invention also provides chimeric molecules comprising a SHAL as described herein attached to an effector (e.g., an epitope tag, a second SHAL, an antibody, a label, a cytotoxin, a liposome, a radionuclide, a drug, a prodrug, a viral particle, a cytokine, and a chelate. In certain embodiments the effector is an epitope tag selected from the group consisting of an avidin, and a biotin. In certain embodiments the effector is a cytotoxin selected from the group consisting of a Diphtheria toxin, a Pseudomonas exotoxin, a ricin, an abrin, and a thymidine kinase. In certain embodiments the effector is a chelate comprising a metal isotope selected from the group consisting

of ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴Cu, ⁵²Fe, ^{52m}Mn, ⁵¹Cr, ¹⁸⁶, Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag. In certain embodiments effector is a chelate comprising an alpha emitter (e.g., bismuth 213). In certain embodiments the effector is a chelate comprising DotA. In certain embodiments the effector is a lipid or a liposome (e.g., a liposome containing a drug).

In still another embodiment, this invention provides a pharmaceutical formulation the formulation comprising a polydentate selective high affinity ligand (SHAL) that specifically binds to a cancer cell as described herein and a pharmaceutically acceptable excipient. In certain embodiments the formulation may be provided as a unit dosage formulation. In certain embodiments the formulation may be provided as a time-release formulation.

This invention also provides a pharmaceutical formulation the formulation comprising a pharmaceutically acceptable excipient and a chimeric molecule comprising a SHAL as described herein. In certain embodiments the formulation may be provided as a unit dosage formulation. In certain embodiments the formulation may be provided as a time-release formulation.

Methods are provided for inhibiting the growth or proliferation of a cancer cell. The methods typically involves contacting the cancer cell (e.g., metastatic cell, tumor cell, etc.) with a polydentate selective high affinity ligand (SHAL) that specifically binds to a cancer cell and/or with a chimeric molecule comprising a polydentate selective high affinity ligand (SHAL) that specifically binds to a cancer cell attached to an effector (e.g., drug, liposome, cytotoxin, radionuclide, or chelator).

In certain embodiments, this invention provides SHALS that specifically bind to a desired target. The target may be any target for which it is desired to create a binding moiety. The SHAL typically comprises two or more ligands joined directly or through a linker where a first ligand that binds to a first site on the target and the second and/or third ligand binds to second and/or third site on the target on same target marker where the first, second and/or third sites are different sites (e.g., all three ligands are capable of simultaneously binding to the target(s)). In certain embodiments the first, second and/or third site is a pocket (or “bump”) on the target(s). In certain embodiments the first, second and/or third sites are on the same target molecule.

This invention also provides various detection methods. In certain embodiments this invention provides a method of detecting a cancer cell. The method typically involves contacting the cancer cell with a chimeric molecule comprising a SHAL that specifically binds to a cancer cell (e.g., to a cancer marker) attached to a detectable label (e.g., gamma-emitter, a positron-emitter, an x-ray emitter, an alpha emitter, a fluorescence-emitter, etc.) and detecting the presence or absence of the detectable label. In certain embodiments the method typically involves contacting a cancer cell with a chimeric molecule comprising chimeric molecule comprising SHAL that specifically binds to a cancer cell (e.g., to a cancer marker) attached to an epitope tag; contacting the chimeric molecule with a chelate comprising a detectable moiety whereby the chelate binds to the epitope tag thereby associating the detectable moiety with the chelate; and detecting the detectable moiety. In certain embodiments the detectable moiety is a radionuclide (e.g., a gamma-emitter, a positron-emitter, an alpha emitter, an x-ray emitter, etc.). In certain embodiments the detecting comprises external imaging and/or internal imaging. In certain embodiments the detectable moiety comprises a metal isotope selected from the group consisting of ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁶⁴¹Cu, ⁵²Fe, ^{52m}Mn, ⁵¹Cr, ¹⁸⁶, Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵Rh, and ¹¹¹Ag. In certain embodiments the chelate comprises DotA. In certain embodiments the epitope tag is an avidin or a biotin.

This invention also contemplates kits for creating and/or using SHALs of this invention and/or chimeric molecules comprising SHALs of this invention. In certain embodiments the kit comprises a container containing a SHAL as described herein and/or containers containing ligands for assembly into a SHAL as described herein. The tether may optionally further include one or more linkers, one or more effectors (chelates, radionuclides, etc.), and the like. In certain embodiments the SHAL is in a pharmacologically acceptable excipient.

In certain embodiments, this invention expressly excludes SHALs where the binding moieties comprising the SHAL are antibodies, single chain antibodies, and the like. In certain embodiments the SHALs are not polyvalent antibodies or polyvalent single chain antibodies. In certain embodiments the ligands comprising the SHALs are not proteins. In certain embodiments, this invention expressly excludes SHALS where the binding moieties comprising the SHAL preferentially and/or specifically bind nucleic acids. In certain embodiments the ligands comprising the SHALs are small organic molecules.

### Definitions

The terms “specific binding” or “preferential binding” refer to that binding which occurs between such paired species as enzyme/substrate, receptor/agonist, antibody/antigen, and lectin/carbohydrate which may be mediated by covalent and/or non-covalent interactions. When the interaction of the two species typically produces a non-covalently bound complex, the binding which occurs is typically electrostatic, and/or hydrogen-bonding, and/or the result of lipophilic interactions. Accordingly, “specific binding” occurs between pairs of species where there is interaction between the two that produces a bound complex. In particular, the specific binding is characterized by the preferential binding of one member of a pair to a particular species as compared to the binding of that member of the pair to other species within the family of compounds to which that species belongs. Thus, for example, a ligand may show an affinity for a particular pocket on an HLA-DR10 molecule that is at least two-fold preferably at least 10-fold, more preferably at least 100-fold, at least 1000-fold, or at least 10000-fold greater than its affinity for a different pocket on the same or related proteins.

The terms “ligand” or “binding moiety”, as used herein, refers generally to a molecule that binds to a particular target molecule and forms a bound complex as described above. The binding may be highly specific binding, however, in certain embodiments, the binding of an individual ligand to the target molecule may be with relatively low affinity and/or specificity. The ligand and its corresponding target molecule form a specific binding pair. Examples include, but are not limited to small organic molecules, sugars, lectins, nucleic acids, proteins, antibodies, cytokines, receptor proteins, growth factors, nucleic acid binding proteins and the like which specifically bind desired target molecules, target collections of molecules, target receptors, target cells, and the like.

The term “small organic molecule” refers to a molecule of a size comparable to those organic molecules generally used in pharmaceuticals. The term excludes natural biological macromolecules (e.g., proteins, nucleic acids, etc.). Preferred small organic molecules range in size up to about 5000 Da, more preferably up to 2000 Da, and most preferably up to about 1000 Da.

The term “ligand library” refers to a collection (e.g., to a plurality) of ligands or potential ligands. The ligand library may be an actual physical library of ligands and/or a database (e.g., a compound database comprising descriptions of a plurality of potential ligands such as the MDL® Available Chemicals Directory, ChemSpider, and the like).

The term “SHAL” refers to a molecule comprising a plurality of ligands that each bind to a different region of the target molecule to which the SHAL is directed. The ligands are joined together either directly or through a linker to form a polydentate moiety that typically shows high avidity for the target molecule. In certain embodiments, the SHAL comprises two or more ligands that bind their target with low affinity (e.g., <10⁻⁶M and/or dissociates within seconds or less) that, when coupled together, form a SHAL that binds the target with high affinity (e.g., >10⁻⁶M, or >10⁻⁷M, or >10⁻⁸M and/or dissociates slowly, e.g., hours to days).

The term “polydentate” when used with respect to a SHAL indicates that the SHAL comprises two or more ligands. The ligands typically bind to different parts of the target to which the SHAL is directed.

The terms “bidentate”, “tridentate”, and so forth when used with respect to a SHAL refer to SHALs consisting of two ligands, SHALs consisting of three ligands, and so forth.

The term “polyvalent SHAL” refers to a molecule in which two or more SHALs (e.g., two or more bidentate SHALs) are joined together. Thus, for example a bivalent SHAL refers to a molecule in which two SHALs are joined together. A trivalent SHAL refers to a molecule in which three SHALs are joined together, and so forth.

A “polyspecific SHAL” is 2 or more SHALs joined together where each SHAL is polydentate and either or both may be polyvalent synthesized (or otherwise generated) so that they have 2 or more targets for each SHAL (set of poly ligands). For example, a SHAL may be synthesized with two or more ligands for the cavities of HLA-DR and cavities on a CDXX, e.g., CD20 or CD22, or all 3, etc. Another example involves joining a MUC-1 SHAL and an antilyphoma SHAL because some lymphomas overexpress traditional HLA-DR and CD receptors and MUC-1 (upregulated). SHAL synthesized with 2 or more ligands for the cavities of HLA-DR and cavities for a chelate, e.g DotA, etc. where in the univalent or bivalent SHAL targets the malignant cell and the univalent or bivalent 2nd module catches a subsequently delivered agent, e.g., DotA chelated radiometal or a prodrug intended to activate the drug transported to the malignant cell by the 1st SHAL.

The term “virtual in silico” when used, e.g., with respect to screening methods refers to methods that are performed without actual physical screening of the subject moieties. Typically, virtual in silico screening is accomplished computationally, e.g., utilizing models of the particular molecules of interest. In certain embodiments, the virtual methods may be performed using physical models of the subject molecules and/or by simple visual inspection and manipulation.

The phrase “target for a SHAL” refers to the moiety that is to be specifically bound by the bidentate or polydentate SHAL.

The phrase “an algorithm found in . . . ”, e.g., “an algorithm found in SPHGEN” refers to an algorithm that is implemented by (found in) the referenced software. The algorithm, however, may be manually, or by a program other than the referenced software and still represent a use of an algorithm found in the referenced software.

The term “pocket” when referring to a pocket in a protein refers to a cavity, indentation or depression in the surface of the protein molecule that is created as a result of the folding of the peptide chain into the 3-dimensional structure that makes the protein functional. A pocket may readily be recognized by inspection of the protein structure and/or by using commercially available modeling software (e.g., DOCK).

The term “cancer markers” refers to biomolecules such as proteins that are useful in the diagnosis and prognosis of cancer. As used herein, “cancer markers” include but are not limited to: PSA, human chorionic gonadotropin, alpha-fetoprotein, carcinoembryonic antigen, cancer antigen (CA) 125, CA 15-3, CD20, CDH13, CD 31, CD34, CD105, CD146, D16S422HER-2, phospatidylinositol 3-kinase (PI 3-kinase), trypsin, trypsin-1 complexed with alpha(1)-antitrypsin, estrogen receptor, progesterone receptor, c-erbB-2, bc1-2, S-phase fraction (SPF), p185erbB-2, low-affinity insulin like growth factor-binding protein, urinary tissue factor, vascular endothelial growth factor, epidermal growth factor, epidermal growth factor receptor, apoptosis proteins (p53, Ki67), factor VIII, adhesion proteins (CD-44, sialyl-TN, blood group A, bacterial lacZ, human placental alkaline phosphatase (ALP), alpha-difluoromethylornithine (DFMO), thymidine phosphorylase (dTHdPase), thrombomodulin, laminin receptor, fibronectin, anticyclins, anticyclin A, B, or E, proliferation associated nuclear antigen, lectin UEA-1, CEA, 16, and von Willebrand's factor.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” or grammatical equivalents herein refer to at least two nucleotides covalently linked together. A nucleic acid of the present invention is preferably single-stranded or double stranded and may generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphosphoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “biotin” refers to biotin and modified biotins or biotin analogues that are capable of binding avidin or various avidin analogues. “Biotin”, may be, inter alfa, modified by the addition of one or more addends, usually through its free carboxyl residue. Useful biotin derivatives include, but are not limited to, active esters, amines, hydrazides and thiol groups that are coupled with a complimentary reactive group such as an amine, an acyl or alkyl group, a carbonyl group, an alkyl halide or a Michael-type acceptor on the appended compound or polymer.

Avidin, typically found in egg whites, has a very high binding affinity for biotin, which is a B-complex vitamin (Wilcheck et al. (1988) Anal. Biochem, 171:1). Streptavidin, derived from Streptomyces avidinii, is similar to avidin, but has lower non-specific tissue binding, and therefore often is used in place of avidin. As used herein “avidin” includes all of its biological forms either in their natural states or in their modified forms. Modified forms of avidin which have been treated to remove the protein's carbohydrate residues (“deglycosylated avidin”), and/or its highly basic charge (“neutral avidin”), for example, also are useful in the invention.

The term “residue” as used herein refers to natural, synthetic, or modified amino acids.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example, Fab molecules may be displayed on phage if one of the chains (heavy or light) is fused to g3 capsid protein and the complementary chain exported to the periplasm as a soluble molecule. The two chains may be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778). Particularly preferred antibodies should include all that have been displayed on phage or yeast (e.g., scFv, Fv, Fab and disulfide linked Fv (Reiter et al. (1995) Protein Eng. 8: 1323-1331).

The term “specifically binds”, as used herein, when referring to a SHAL or to a biomolecule (e.g., protein, nucleic acid, antibody, etc.), refers to a binding reaction that is determinative of the presence of the SHAL or biomolecule in a heterogeneous population of molecules (e.g., proteins and other biologics). Thus, under designated conditions (e.g., binding assay conditions in the case of a SHAL or stringent hybridization conditions in the case of a nucleic acid), the specified ligand or SHAL preferentially binds to its particular “target” molecule and preferentially does not bind in a significant amount to other molecules present in the sample.

An “effector” refers to any molecule or combination of molecules whose activity it is desired to deliver/into and/or localize at a target (e.g., at a cell displaying a characteristic marker). Effectors include, but are not limited to labels, cytotoxins, enzymes, growth factors, transcription factors, drugs, lipids, liposomes, etc.

A “reporter” is an effector that provides a detectable signal (e.g., is a detectable label). In certain embodiments, the reporter need not provide the detectable signal itself, but may simply provide a moiety that subsequently may bind to a detectable label.

The term “conservative substitution” is used in reference to proteins or peptides to reflect amino acid substitutions that do not substantially alter the activity (specificity or binding affinity) of the molecule. Typically, conservative amino acid substitutions involve substitution of one amino acid for another amino acid with similar chemical properties (e.g., charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The terms “epitope tag” or “affinity tag” are used interchangeably herein, and usually refers to a molecule or domain of a molecule that is specifically recognized by an antibody or other binding partner. The term also refers to the binding partner complex as well. Thus, for example, biotin or a biotin/avidin complex are both regarded as an affinity tag. In addition to epitopes recognized in epitope/antibody interactions, affinity tags also comprise “epitopes” recognized by other binding molecules (e.g., ligands bound by receptors), ligands bound by other ligands to form heterodimers or homodimers, His6 bound by Ni-NTA, biotin bound by avidin, streptavidin, or anti-biotin antibodies, and the like.

Epitope tags are well known to those of skill in the art. Moreover, antibodies specific to a wide variety of epitope tags are commercially available. These include but are not limited to antibodies against the DYKDDDDK (SEQ ID NO: 04) epitope, c-myc antibodies (available from Sigma, St. Louis), the HNK-1 carbohydrate epitope, the HA epitope, the HSV epitope, the His₄, His₅, and His₆ epitopes that are recognized by the His epitope specific antibodies (see, e.g., Qiagen), and the like. In addition, vectors for epitope tagging proteins are commercially available. Thus, for example, the pCMV-Tagl vector is an epitope tagging vector designed for gene expression in mammalian cells. A target gene inserted into the pCMV-Tagl vector may be tagged with the FLAG® epitope (N-terminal, C-terminal or internal tagging), the c-myc epitope (C-terminal) or both the FLAG (N-terminal) and c-myc (C-terminal) epitopes.

### DETAILED DESCRIPTION

This invention pertains to the development of a new class of binding molecules that may be used to specifically bind just about any target molecule(s). This class of binding molecules are referred to herein as Selective High Affinity Ligands or “SHALs”. The SHALs may be used in a manner analogous to antibodies in a wide variety of contexts that include, but are not limited to capture reagents in affinity columns for purification of biological or other materials, binding agents in bio sensors, agents for the assembly of nanoparticles or nanomachines, diagnostics, and therapeutics.

SHALs may also be used to detect molecular signatures that may distinguish between various pathogen types or strains. SHALs also have use in biodefense applications for the detection of unique protein signatures present in toxins and on the surfaces of pathogenic organisms and to distinguish these biothreat agents from naturally occurring non-hazardous materials. Because the SHALs may be relatively stable when exposed to the environment, they are particular well suited for use in biosensors for biodefense, diagnostic, and other applications.

In certain preferred embodiments, the SHALs are used in the diagnosis and/or treatment of cancers. In such embodiments, the SHALs are directed to unique and/or specific sites (e.g., cancer-specific markers) on the surfaces of cancers (e.g., various malignant cells).

In certain embodiments, the SHALs may have a therapeutic effect when administered per se (e.g., in a manner analogous to the antibody therapeutic Herceptin™). A SHAL, like an antibody, may have a direct effect on a malignant cell that leads to cell death because the SHAL serves as an agonist against a normal pathway, thereby initiating or blocking critical cell functions and leading to malignant cell death. A SHAL may also act as a vaccine because it provides malignant cell identification either because it represents an aberrant cell surface marker or enhances a usual malignant cell marker.

In addition, or alternatively, the SHAL(s) may be used as targets (when bound to the targeted cell), or as carriers (targeting moieties) for other effectors that include, but are not limited to agents such as cytotoxic agents, markers for identification by the immune system, detectable labels (for imaging), and the like.

Radioisotopes are attractive examples of cytotoxic effectors that may be attached to the carrier SHAL to selectively deliver radiotherapy to the malignant cell(s). This therapy may be administered as single agent therapy, or in combination with marrow reconstitution in order to achieve greater dose intensity, or other drugs that may enhance the radiation effects on the malignant disease. Although there are many different drugs, chemotherapeutic, biological and otherwise, that may be combined with the SHAL, taxanes are one attractive example.

Examples of cytotoxic agents include radioisotopes, immunotoxins, chemotherapeutics, enzyme inhibitors, biologicals, etc. Interesting examples include apoptotic signals and enzymes such as the caspases. Radioisotopes represent interesting cytotoxic agents that have been shown to be effective in conjunction with antibody antigen and ligand-receptor systems. For treatment purposes, according to the present invention, it is considered that, in some embodiments, labeling with a particle emitter such as beta−, beta+ (positron) are preferable. In some cases, labeling with an alpha emitter or Auger-electron emitter is appropriate. There are many examples of therapeutic radioisotopes including yttrium-90 or iodine-131 that are of considerable current interest.

For certain imaging purposes, according to the present invention, it is considered that technetium-99, indium-111, iodine-123, or iodine-131 are attractive for single photon imaging and that beta+ (positron) emitters such as copper-64, yttrium-86, gallium-68, etc. are particularly likely to be very attractive when attached to a SHAL for diagnostic purposes

A SHAL consists of two or more ligands (also referred to as binding moieties) linked together directly or through a linker to generate a core “polydentate” molecule (SHAL) that has been designed to specifically bind to essentially any desired target (e.g., unique or specific sites (pockets) on an intended target malignant cell surface molecule). The ligands (binding moieties) comprising the SHAL may include essentially any moiety capable of binding a site on the target. Such binding moieties may include, but are not limited to various chemicals (e.g., small organic molecules), proteins, sugars, carbohydrates, lectins, lipids, metals, nucleic acids, peptide and nucleic acid analogues, and the like.

Although not required, the individual ligands comprising the SHAL often have relatively low affinity (e.g., less than about 10⁻⁶ M) for the target. In contrast, the polydentate SHAL (comprising a plurality of ligands) typically shows relatively high avidity (e.g., greater than about 10⁻⁶M, preferably greater than about 10⁻⁸ or 10⁻⁹ or 10⁻¹⁰, still greater than about 10⁻¹¹ M, and most preferably greater than about 10⁻¹²M).

In certain embodiments, where the target to which the SHAL is to be directed is a protein, the ligands comprising the SHAL may be selected to bind certain non-functional sites on the protein. A protein often has a number (few to >50) of “pockets” or cavities distributed across its surface. These cavities are produced as the protein chain is folded into a three-dimensional structure to make the protein functional. This observation makes it possible to consider designing SHALs that exhibit much greater binding specificity for a given protein than previously possible. By linking together two moieties that bind to unique pockets on the surface of a protein with only micromolar affinities, it is possible to design polydentate molecules (SHALs) that bind with nanomolar to picomolar affinities and are highly selective and do not cross react with other functionally related molecules. For proteins with a known or predicted structure, computational methods may be used to generate a three-dimensional map of the molecular surface and identify suitable sized pockets that are structurally unique for that protein as described herein.

Databases containing the structures of known small molecules may be screened for their ability to bind into pockets on the target protein using a “docking” program. The top candidates may then be tested using a variety of experimental techniques as described herein to identify the molecules that actually bind to the protein as well as those that bind to the correct site. Pairs or triplets of the ligands (e.g., one from each set) may then be attached to opposite ends of an appropriate length linker using solid or solution phase chemistry to generate bidentate SHALs or tridentate SHALs. The highest affinity and most selective SHALs may then be identified by conducting conventional binding studies.

It was a surprising discovery that linking together two or more, small ligands that bind weakly to target (e.g., a protein) and exhibit little or insufficient selectivity may result in the production of a molecule that binds to its intended target (e.g., target protein) three to six or more orders of magnitude more tightly and with high selectivity.

Without being bound to a particular theory, it is believed that while the presence of two or more ligands in the SHAL would be expected to increase the odds that the molecule might bind to a wider variety of proteins, this non-specific binding may be weak (approximately the same as the free ligand) and those molecules attached to non-target proteins via only one of the ligands will not remain bound long. The enhanced affinity and selectivity observed when both ligands in a bidentate SHAL or all the ligands comprising a polydentate SHAL bind to their respective targets (e.g., pockets on a target protein) is derived from three factors that relate to the nature of the SHAL-target interaction: First, the presence of the linker prevents the individual ligands comprising the SHAL that dissociate from their target from diffusing away from the target surface, increasing significantly the rate at which the free ligand rebinds. Second, the reduced off rate of release of the bidentate or polydentate SHAL is dictated by the fact that the probability that both (or all) ligands comprising the SHAL will simultaneously release from their target is substantially lower than the probability that either one will release/The spacing between the ligands comprising the SHAL which is determined by the attachment chemistry (e.g., the linker), allows the ligands comprising the SHAL to bind simultaneously to their target only if the ligands are separated by the correct distance. If either ligand in the SHAL binds independently to another target, its low affinity (1-10 micromolar affinity is typical for the ligands that's been identify) would result in the ligand falling off rapidly (the off-rate would be high). Thus, the only situation in which the bidentate or polydentate SHAL would bind tightly to the intended target (nanomolar affinity or higher) would be when both ligands comprising the SHAL bind simultaneously to the target molecule (e.g., target protein). Once both or all ligands are bound, the off-rate of the entire molecule (the SHAL) would be reduced dramatically. If the ligand binding sites are selected properly (e.g., by targeting regions that vary in amino acid sequence or structure in the case of a protein target), it becomes highly improbable that identical sites will be found on another target separated by the same distance. If this extremely unlikely event were to occur, an additional ligand binding site (e.g., a third binding site adjacent to Site 1 and -2) may be identified, and an additional ligand may be incorporated into the SHAL (e.g., to create a tridentate, quadridentate, etc. SHAL).

SHALs have certain advantages over antibodies, particularly in therapeutic and/or diagnostic applications. Typically, SHALs are considerably smaller than antibodies. They are consequently able to achieve greater tumor penetration. In certain embodiments, they are also able to cross the blood brain barrier, e.g., for the treatment of brain tumors. It is believed that the SHALS are also often less immunogenic than antibodies, and are often cleared from the circulation less rapidly.

In certain embodiments SHALs described herein are typically polydentate, i.e., the SHAL comprises two or more ligands, that are joined together directly or through one or more linkers. In certain embodiments the ligands bind to different parts of the target (e.g., different epitopes on a single protein) to which the SHAL is directed. In certain embodiments the ligands bind to different molecules, e.g., different cancer markers on a cancer cell, different proteins comprising a receptor, and the like. In certain embodiments polyspecific SHALS may be used for crosslinking the same or different antigens on the same cell thereby enhancing the signal transduction, or for pretargeting, e.g., where one SHAL is designed to target malignant cells and is attached to other SHALs designed to “catch” a subsequently administered carrier of a cytotoxic agent (e.g, chelated radiometal, etc.), to recruit an immunologically active cell (e.g., macrophage, T-cell, etc.) to the site, to activate a prodrug on the targeted SHAL, and so forth.

In certain embodiments this “multiple specificity” is achieved by the use of polyvalent SHALs. Polyvalent SHALs are molecules in which two or more SHALs (e.g., two or more bidentate SHALs) are joined together. The different SHALs comprising the polyvalent SHAL may be directed to the same or different targets, e.g., as described above.

I. Construction of SHALs.

SHALs of this invention are created by identifying ligands (binding moieties) that bind, and in some embodiments, that specifically (or preferentially) bind, different regions of the target molecule or molecules. Ligands binding different regions of the target molecules are then joined directly or through a linker to produce a bidentate SHAL comprising two different binding moieties or a polydentate SHAL comprising two or more different biding moieties. The SHAL may then be screened for its ability to bind the intended target.

The initial identification of ligands that bind different regions of the target may be accomplished using virtual in silico methods (e.g., computational methods) and/or empirical methods, e.g., as described herein.

Once two or more suitable ligands (binding moieties) are identified they can, optionally be screened (validated) for the ability to bind the target at different sites. Suitable binding ligands may then be coupled together directly or through a linker to form a bidentate or polydentate SHAL that may then, optionally, be screened for the ability to bind the target.

A) Target Selection.

Virtually any molecule, receptor, combination of molecules may serve as a target for a SHAL. Target selection is determined by the application for which the SHAL is intended. Thus, for example, where the SHAL is to be incorporated into an affinity column (e.g., to purify a protein or nucleic acid) the target is the molecule (e.g., protein, nucleic acid, etc.) that is to be purified using the affinity column comprising the SHAL.

In certain embodiments, SHALs may be generated for cell surface membrane target proteins that influence intracellular functions, thereby promoting these functions (agonist) or inhibiting these functions (antagonist) by blocking other molecular binding or causing an inhibitory or enhanced intracellular signal, e.g, phosphokinase signaling. SHALs may be generated for cell surface membrane target proteins such as antigens and antibodies that may be internalized into the cell. In common with antibodies that target internalizing antigens and peptide ligands that target internalizing receptors, these SHALs will be internalized and in the cell where they may have agonist or antagonist effects on critical cell functions such as protooncogenes, phosphokinases, lysosomes and DNA/RNA/mRNA because of their agonist or antagonist functions or because they deliver a toxin or radioisotope payload. There are several advantages of SHALs over antibodies and peptide ligands. They include small size and the range of charge that may be used to permit free movement into and within the cell when an intracellular molecule is the primary target.

SHALS may be used to preferentially select specific cells by their membrane targets and, upon dissociation from the targeted cell surface membrane may freely move across the cell surface membrane to access the inside of the cell. The SHAL may be made multi-specific so that when it is internalized, or when it dissociates and penetrates the cell surface membrane, the second specificity may permit targeting of internal cell molecules such as phosphokinases, lysosomal enzymes, hormone receptors, gene and proto oncogene protein products, DNA/RNA/mRNA, and the like. In certain embodiments, uni-specific but multivalent SHALs may be generated that both target call surface molecules and cross-link these molecules leading to enhanced biologic effects that have been described for cross-linked antibody-antigen systems.

In contrast to antibodies and peptide ligands that typically cannot directly and readily penetrate cell surface membranes, because of the small size of SHALs, and the ability to select the hydrophobic or hydrophilic character of the SHAL, SHALs may be produced that are capable of penetrating the cell membrane and various intracellular compartments. This makes it possible to generate SHALs specifically for the purpose of targeting intracellular molecules of importance to cell function, such as proto oncogenes, phosphokinases, lysosomal and other enzymes, DNA/RNA/mRNA, etc. This capability makes it possible to target entire classes of intracellular molecules of critical importance to cell function, a capability not previously achievable by specific targeting molecules. In addition to direct effects of these SHALs, they may be used as carriers of payloads, as described herein.

Many signaling pathways are susceptible to interference by the one (directly intra cellular) or two step (membrane targeting followed by internalization) SHAL targeting of intracellular molecules. These include, but are not limited to such key pathways as “G” protein signaling and tyrosine specific protein kinase activity e.g., EGFR, Neu, etc. Multiple hormone receptor interventions may also be targeted by SHALs to create a change in cell function and/or in cell growth. Hormone or enzyme targets may be bound and the function blocked. Hormone receptor blocks that may be useful include the use of SHALs to block the binding of estrogen like molecules to the ER (estrogen receptor) and similar effects to AR (androgen receptor). This would in turn interfere with DNA binding of the complex, with the resulting interference in hormone sensitive tumor cell growth and viability.

This invention also contemplates the use of SHALS to treat infectious diseases (e.g., AIDs, influenza, etc.) by either primary binding of the infectious agent and/or by blocking cell invasion, and/or by blocking metabolic pathways critical to propagation of the infectious agent (e.g., by blocking CCR5 to prevent HIV infection of cells).

Where the target to which the SHAL is to be directed comprises a protein, in certain embodiments, at least one of the ligands (binding moieties) comprising the SHAL bind to a pocket in the protein. In certain embodiments, where at least two of the binding moieties comprising the SHAL bind to pockets of the protein and those two ligands bind to different pockets. In certain embodiments, all of the ligands comprising the SHAL bind to pockets in the target protein. This is not to suggest that all ligands comprising the SHAL must bind to protein pockets. Certain embodiments are contemplated wherein one ligand binds to a pocket and another ligand binds to a region that is not a pocket or where none of the ligands bind to a pocket.

The empirical approaches described herein may also be used to accelerate the process of SHAL development. IN this circumstance, modeling and other analytical steps may be performed after empirical development of a SHAL as desired for better understanding the SHAL and/or to guide subsequent generations of SHAL development.

In certain embodiments, additional ligand(s) beyond one or two chosen for docking sites in the epitopic region of interest are chosen for docking sites outside the region of interest on the target. This provides greater selectivity and “effective” affinity. In the case of a multimeric target, for example, HLA-DR, additional ligands may be directed to the same or to a different subunit of the target. Specifically, in the case of HLA-DR, one or two ligands may be directed to known docking sites in the epitopic region defined for Lym-1 monoclonal antibody reactivity and additional ligands may be chosen for docking sites in the same or a different multimer, for example, the beta subunit or the alpha subunit of HLA-DR.

Other examples are SHALs with ligands chosen to react with the tandem repeats of mucins, such MUC-1 as described above. In this instance, the core protein is repetitive at ten mer intervals so that SHALs of similar or identical nature may be joined to provide multivalent linkage to identical or similar but different repeats of known (or unknown) distance. Alternatively, the SHAL may have a third ligand that is identical to one of the initial ligands but linked at a distance to docking sites at a remote region of the core tandem repeat of MUC-1.

Also, docking sites may be protrusions in addition to cavities, although the latter are likely to confer greater (affinity) by virtue of the potential for more contact interactions.

B) Compounds (Putative Ligands/Binding Moieties) to be Screened.

Virtually any agent may be screened for its ability to bind a target and thereby for its suitability for incorporation into a SHAL according to the methods of this invention. Such agents include, but are not limited to nucleic acids, proteins/peptides, nucleic acid or peptide analogs, metals, sugars, polysaccharides, glycoproteins, lipids, lectins, large and small organic molecules, antibody CDRs, and the like.

In one preferred embodiment, high throughput screening methods involve providing a combinatorial chemical library containing a large number of potential ligands (binding moieties). Such “combinatorial chemical libraries” are then screened in one or more assays, as described herein to identify those library members (particular chemical species or subclasses) that display the desired binding activity. The compounds thus identified may serve as a component of a SHAL.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of chemical building blocks called amino acids in every possible way for a given compound length (i.e., the number of amino acids in a polypeptide compound). Millions of chemical compounds may be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) J. Med. Chem., 37(9): 1233-1250).

Preparation of combinatorial chemical libraries is well known to those of skill in the art. Such combinatorial chemical libraries include, but are not limited to, peptide libraries (see, e.g., U.S. Pat. No. 5,010,175; Furka (1991) Int. J. Pept. Prot. Res., 37: 487-493; Houghton et al. (1991) Nature, 354: 84-88). Peptide synthesis is by no means the only approach envisioned and intended for use with the present invention. Other chemistries for generating chemical diversity libraries may also be used. Such chemistries include, but are not limited to: peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a beta-D-glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

In certain embodiments, the initial screen for ligands (binding moieties) with which to build the SHAL may be performed as a virtual in silico screening method and, in this case, it is not necessary to have the physical compounds in hand. In such instances chemical structure databases provide a wide range of moieties that may be screened for their suitability for inclusion in a SHAL as described herein.

Chemical structure databases are well known to those of skill in the art. For example, the MDL® Available Chemicals Directory (MDL ACD) is presently the largest structure-searchable database of commercially available chemicals in the world and is available from MDL Information Systems, Inc., San Leandro, Calif. This database is merely illustrative and not intended to be limiting. Other chemical structure databases (e.g. ChemSpider, ZINC, etc.) are well known to those of skill in the art and include, but are not limited to various organic molecule, peptide, carbohydrate or nucleic acid structural databases.

C) Computational Identification of Ligands that Bind the Target.

Using a virtual in silico approach, computational methods may be used to characterize (e.g., model) the target (e.g., target protein) and to identify molecules (binding moieties) that are expected to specifically bind to certain regions of the target. The use of computational methods to identify molecules that specifically bind to a particular target is often referred to as “DOCKING”.

Docking methods are well known to those of skill in the art. Two approaches to docking are “rigid molecule docking” in which the molecules involved are treated as rigid objects that cannot change their spatial shape during the docking process, and soft (flexible) docking where the molecules are (computationally) allowed to change shape as they dock.

There are several physical and chemical forces that interact between the two molecules. These forces are used to define various docking scores that measure quality of each solution. These scores take into account the strength of these forces and the plausibility of the docking solution. The most significant forces typically considered in docking algorithms include electrical forces, van der Walls forces, and hydrogen bonds.

The docking problem is often formally stated as follows: Let A,B be two rigid molecules (e.g., the target and the potential ligand that is to bind the target) with their geometric representation in R3. We would like to find a rigid transformation T:R3→R3 such that the contact surface between T·A and B is maximal. The contact surface is typically defined as the surface where the distance between the molecules is smaller than a given threshold. Typically docking algorithms try to achieve a contact surface which is “large enough” instead of “maximal” and that we try to maximize not the size of the contact surface but a score measuring the quality of the proposed docking solutions. These two parameters are correlated but are not equivalent.

1. Rigid Docking.

One approach to the rigid docking algorithm was described by Kuntz et al. (1982) J. Mol. Biol., 161: 269-288. The Kuntz et al. algorithm is primarily used in solving ligand-protein docking while trying to focus on ‘interesting’ sites on the surface of the molecules. The basic stages of the algorithm involve first computing the molecular surface using Connolly's method (see, e.g., Connolly (1983) J. Appl. Crystallography, 16: 548-558; Connolly (1983) Science, 221: 709-713)). This produces a set of points on the “smoothed” molecular surface with their normals. Then a “sphere generator” (e.g., SPHGEN) is used to create a new representation of the molecular surface of the target (e.g., protein) and the ligand using “pseudo-atoms” and then uses this representation to find plausible docking sites on the molecular surface—these docking sites that SPHGEN is looking for are cavities in the surface of the receptor.

SPHGEN typically consists of the following five stages: First, for each pair on Connolly points pᵢ, pⱼ a sphere passing through this pair is placed such that its center is on one of the points normal. The algorithm then defines Sₙₒᵣₘ(i)={Spheres whose center is on the normal of pᵢ}. Assuming that there are Connolly points, then for each 1<=i<=n and pᵢ is on the surface of the target, we throw away all the spheres in Sₙₒᵣₘ(i) and leave only the one with the smallest radius. This throws all the spheres that penetrate the surface of the target. The algorithm then typically only leaves the spheres where Theta (e.g., the angle between the pi normal and the radius from pⱼ to the center of the sphere)<90°. Otherwise the points that define the sphere, pi and pj, are too close to each other and therefore are not located in a cavity on the target's surface. Then the algorithm typically for each atom leaves only the sphere with the maximal radius. This step leaves only the spheres that ‘touch’ the surface of the atom. Finally, if the points that define the sphere, pi and pᵢ, belong to two different atoms, and the distance between these atoms on the molecular sequence is less than 4—the sphere is discarded. This is done because the length of a curve on an Alpha-helix is 3.6 and these sites are typically not to be treated as possible docking sites.

The remaining spheres are called pseudo-atoms. The next stage looks for clusters of intersecting pseudo-atoms. The existence of this kind of cluster indicates the existence of a cavity in the molecular surface, which is considered to be a good docking site.

After all this is performed on the target the same is done on the ligand, but this time we take the points and the vectors opposite to their normals, in order to create the spheres inside the surface instead of outside the surface. The result of SPHGEN on the target is sometimes called the ‘negative image’ and on the ligand it's called the ‘positive image’. In certain embodiments, the vectors with respect to the ligand and the target may be reversed (e.g., to find elements of the target that dock within cavities in the ligand).

“Matching” is then typically performed. In this operation, for each docking site, the algorithm tries to find a transformation T that gives a good correspondence between the centers of the pseudo-atoms of the target to those of the ligand (in some cases, the centers of the real atoms of the ligand are used, instead of the centers of its pseudo-atoms). In some versions of DOCK, clusters of pseudo-atoms are separated into sub-clusters in order to improve the complexity of this stage. One way of doing this is to discard the largest sphere in the cluster, which sometimes causes the cluster to be divided into two sub-clusters.

In the matching problem of rigid docking a search is performed to find a translation and rotation of one molecule, such that good matching between the interesting points in both molecules is formed. In certain embodiments, the distances between the point used instead of the points locations: For each molecule, the target (T) and the ligand (L), an appropriate distance matrix is defined—d^{T}_{i,j} and d^{L}_{i,j}, respectively. A search is then performed to try to find two subsets in T and L such that their distances are the same, with some tolerance of error. These two subsets define two subgraphs with almost similar distances between their vertices. This may be done using a method similar to the interpretation tree by Grimson and Lozano-Perez.

Another way of solving the matching problem is to find a “large enough” clique in a matching graph. If there are n points in the target and m points in the ligand, the matching graph has n*m vertices where each vertex represents a point from the target and a point from the ligand. Let G=(V,E) be the matching graph and let u,v be vertices in V where u represents u_{L} and u_{T} (points in the ligand and the target, respectively) and v represents v_{L} and v_{T}. An edge e=(u,v) will be added to E only when ABS [d^{L}(u_{L},v_{L})−d^{T}(u_{T},v_{T})]<tolerance. Therefore, a clique in the matching graph defines subsets of points in the ligand and the receptor with similar distances.

In order to evaluate the quality of the match a score is calculated. The score preferably takes into account the size of the contact surface between the molecules and typically does not allow one molecule to penetrate the other. The DOCK algorithm uses a cubic grid that fills the binding site and every cell in this grid has a score according to its distance from the centers of the receptor's atoms: 1 if the distance is 2.8 Å-4.5 Å, −127 if the distance is less than 2.8 Å, and 0 if the distance is more than 4.5 Å. (In some cases, the distance 2.8 Å is replaced by 2.4 Å). For each proposed transformation, the position of the ligand's points (i.e. the centers of its atoms or pseudo-atoms) in the grid are calculated and the score is calculated as the sum of scores of these points. Additional or alternative scores may be used various versions of the algorithm. For example, in one version a van der Waals energy score may be calculated for the transformations that have good matching scores.

Construction of a computer model of HLA-DR 10. Using the crystal structures that have been determined for four closely related human HLA-DR molecules (HLA-DR 1-4), the identification of unique “pockets” on surface of the protein, the identification of ligands that bind certain unique pockets and the construction of a SHAL using these ligands is illustrated herein in the examples.

The foregoing description is intended to be illustrative of one approach to rigid docking and is not intended to be limiting. Other approaches are known to those of skill in the art. It is noted that the SPHGEN and DOCK programs are commercially available (e.g., directly from the University of California and various commercial manufacturers of software).

2. Soft (Flexible) Docking.

Soft docking algorithms are also well known to those of skill in the art (see, e.g., Jiang and Kim (1991) J. Mol. Biol., 219: 79-102; Katchalski-Katzir et al. (1992) Proc. Natl. Acad. Sci. U.S.A., 89(6): 2195-2199, etc.).

In the method described by Jiang and Kim, supra, an enumeration on the 6-dimensional space of rigid transformation is performed and these transformations are given scores according to their energetic value. Both molecules are placed on a grid and the matching is evaluated using the distances between grid cells, the number of penetrations and the directions of the points' normals. The algorithm works on the output of Connolly's algorithm and works on the entire molecular surface (i.e. no cavities are looked for—as opposed to the DOCK algorithm). In order to decrease the enumeration, the algorithm typically uses two resolutions—low and fine. The low-resolution uses ^{˜}0.3 points per square angstrom, and the fine resolution uses ^{˜}1 point per square angstrom.

Each cell in the grid is marked as “surface” (if it contains at least one Connolly point) or “volume” (if it doesn't contain any Connolly point). Usually, each surface cell contains 2-3 Connolly points.

An enumeration on the rotations of one of the molecules (usually smaller one) is performed. For each rotation the following is performed: The surface and volume cell of the molecule is calculated. Assuming that there's at least one pair of surface cells (one from each molecule) that are matched by the transformation, an enumeration on all of these pairs is performed. For each pair the transformation is calculated and it is evaluated by checking the directions of the normals, the number of surface-to-surface matches and the number of penetrations. The good transformations are those who have a small number of penetrations and a lot of surface-to-surface matches. This is done first in low resolution and the best results are calculated again in fine resolution with the addition of an approximated energetic score. The approximated energetic score is calculated according to the number of “favorable” and “unfavorable” interactions. There are several categories for the atoms of each molecule and combinations of these categories are marked as “favorable” if they have a good contribution to the energetic plausibilty of the match, or “unfavorable” otherwise.

For example, it is unfavorable that an atom with positive charge is placed near another atom with positive charge, but it is favorable if two atoms are adjacent if one of them is an H-donor and the other is an H-acceptor.

The approach of Katchalski-Katzir et al., supra, is to enumerate on the possible translations, while using FFT to calculate the matching score efficiently. Similar to the previous algorithm, both molecules are placed on a 3-dimensional grid, but here 3 types of grid cells are defined—“volume”, “surface” and “intermediate”. If the molecules are A and B, the matrices A_{l,m,n} and B_{l,m,n} are defined as follows (l,m,n are the grid coordiantes): A_{l,m,n}={1—(l,m,n) is a “surface” cell, q—if (l,m,n) is an “intermediate” cell, 0—otherwise}, and B_{l,m,n}={1—if (l,m,n) is a “surface” cell, r—if (l,m,n) is an “intermediate” cell, 0—otherwise}.

In certain embodiments, parameters are chosen such that q<0 and r>0 while |q| is large and is small. The scalar product of these matrices may be efficiently calculated using FFT thus improving the algorithm's performance considerably.

Again, it is noted that the foregoing description is intended to be illustrative of one approach to rigid docking and is not intended to be limiting. Other approaches are known to those of skill in the art. For example, additional approaches/programs include, but are not limited to: FlexX from Tripos (http://www.biosolveit.de/FlexX/) which is commonly used for high-throughput screening. It uses an empirical scoring function. It allows for flexible docking by rotating around torsional bonds. It is sold as a module of the Sybyl program (distributed by Tripos, Inc., St. Louis). GOLD from CCDC (http://www.ccdc.cam.ac.uk/prods/gold/) which uses a genetic algorithm to generate conformers for a ligand. It also enables customization of the torsional energy within smaller fragments of the molecule and may accommodate local protein flexibility. Autodock UCSD (http://www.scripps.edu/pub/olson-web/doc/autodock/) uses a Lamarckian genetic algorithm to generate conformers for a ligand. AutoDock is best used when there are only a few ligands and the binding energies need to be more accurate. Some good reviews on Docking include Lyne (2002) Drug Discovery Today. 7 (20): 1047, and Taylor et al. (2002) J. Computer-Aided Mol. Design., 16: 151.

D) Empirical Approaches and Verification of Ligand Binding.

The use of computational methods to identify ligands for use in the construction of a SHAL requires at least some information regarding the structure of the target molecule(s). This invention also contemplates the use of methods that require no knowledge regarding the structure of the target to which the SHAL is to be directed.

In certain “empirical” embodiments, individual ligands or libraries of ligands are screened against the target molecule(s) and/or cells, bacteria, viruses, etc. displaying the target molecule(s) to identify ligands that bind the desired target (at least low affinity). Ligands are identified that bind to different regions of the target molecule(s). In certain embodiments, ligands are identified that bind to different regions of the target molecules and that do not exclude each other from such binding.

Ligands that may simultaneously bind to the target without excluding each other may then be joined together, directly or through a linker, to create a polydentate SHAL which can, optionally, be subsequently screened for the ability to bind to the target molecule(s), e.g., at high affinity.

In addition to use in empirical approaches for ligand identification, physical screening methods are also desirable for validating binding of ligands identified using the virtual in silico approaches discussed above. In addition, it may be desirable to additionally determine the binding orientation of two or more ligands, e.g., to confirm that the ligands bind to different sites on the target and/or to estimate spacing when the ligands are incorporated into a SHAL.

Assays for detecting the binding of one or more ligands to a target are well known to those of skill in the art. For example, in one simple embodiment, the ligands may be labeled with a detectable label and contacted with the target molecule(s) which are immobilized on a substrate. After a wash, detection of the labels in association with the immobilized target molecule(s) indicates that the ligands bind to the target. In certain embodiments, different ligands may be labeled with different labels (e.g., different color fluorescent labels), and the simultaneous binding of multiple ligands may be visualized.

Alternatively, competitive binding assays may be performed. In such assays the target molecule(s) are contacted with one ligand known to bind the target. The target is also contacted with the “test” ligand and the ability of the test ligand to bind to the target in the presence of the first ligand is evaluated.

Fluid phase assays may also be performed. For example, the ligand(s) and the targets may be labeled with different labels. The ligands may be contacted to the target molecule(s) and binding of the two may readily be evaluated, e.g., using a flow cytometer. Flow cytometry methods are well known to those of skill in the art (see, e.g., Omerod (1994) Flow Cytometry: A Practical Approach. IRL Press, Oxford.; Shapiro Practical Flow Cytometry. 3rd Edition. Alan R Liss, Inc.; Givan (1992) Flow Cytometry. First Principles. Wiley-Liss, New York; Robinson (1993) Handbook of Flow Cytometry Methods, Wiley-Liss, New York, and the like).

Determination of ligand binding and orientation may also be determined using a number of different methods. These include, but are not limited to Saturation Transfer Difference nuclear magnetic resonance (Mayer and Meyer (1999) Angew Chem Int Edit, 38:1784-1788) and Transfer NOE (trNOE) nuclear magnetic resonance (NMR) spectroscopy (Henrichsen et al. (1999) Angew Chem Int Edit., 38:98-102; Cosman et al. (2002) Chem Res Toxicol 15: 1218-1228). These methods may be used to screen the ligands in mixtures of several to several hundred per experiment to determine which ligands bind to the target molecule(s) e.g., under biologically relevant conditions and to determine which ligands bind to the same (or different) sites. Diffusion experiments (Lin et al. (1997) J. Organic Chem., 62: 8930-8931) may also be performed with those ligands that have been determined to bind in order to assess the relative binding affinity of each compound.

Other approaches to detecting binding of the ligands to the target molecule(s) include, but are not limited to surface plasmon resonance (BIAcore assay), saturation transfer difference nuclear magnetic resonance spectroscopy, other nuclear magnetic resonance spectroscopy measurements, mass spectrometry, capture microarrays, bead-based library assays, and other physical binding assays.

The foregoing assays are intended to be illustrative and not limiting. Using the teaching provided herein numerous other assays for detecting ligand binding to the target molecule(s) will be known to those of skill in the art.

Following the identification of a set of ligands that bind to the target molecule(s) (e.g., HLA-DR10), competition experiments may be performed, e.g., by NMR to determine if they bind to one of the pockets comprising the target molecule(s) (e.g., in the case of HLA-DR10, to one of the pockets encompassing the Lym-1 epitope.

As indicated above, this may readily be accomplished by preparing a complex between the target and a known binding ligand and determining if a second ligand may bind the complex. Thus, for example, the case of HLA-DR10 target, a Lym-1:HLA-DR10 complex may be prepared and the set of ligands that bind to HLA-DR10 may be re-tested to determine if they will still bind to the protein when the Lym-1 antibody is bound.

Those ligands that no longer bind to the Lym-1:HLA-DR10 complex may be identified (these ligands bind to the unique sites that distinguish HLA-DR10 from the other HLA-DR molecules) and used in a second set of competition experiments to identify those molecules that bind to different sites within the Lym-1 epitope. In experiments conducted with pairs of ligands, transfer nuclear Overhauser effects (trNOE) that occur between the bound ligands and the HLA-DR10 protein (Cosman et al. (2002) Chem Res Toxicol 15: 1218-1228), in the absence of the Lym-1 antibody, may be used to identify those ligands that bind to the same and different sites. Bound ligands exhibit negative NOE signals, while unbound ligands have positive signals (Id.). If both ligands in the pair are observed to be bound to the protein at the same time, the results will indicate that the two ligands must bind to different sites. The screening thus may readily identify sets of ligands that bind to different sites within the target molecule(s) (e.g., to different sites (Site 1 and Site 2) within the Lym-1 epitope of HLA-DR10).

After sets of ligands have been identified that to bind to different sites on the target, the orientation of the ligands in the binding sites can, optionally, be further evaluated using classical molecular dynamics simulations. The methods of molecular dynamics simulations are clearly described in the Examples. For example, for each ligand, one to three orientations within the binding pocket may be simulated for 500 psec. This will help determine which functional groups on the ligands are likely to be in contact with the target and which functional groups are accessible by solvent. This information may be used to identify analogs with modified or different functional groups that may be tested for their ability to bind to the target and confirm that a particular functional group may be used as the site for linker attachment without disrupting the binding of the ligand to the target.

The use of these approaches to identify ligands that bind to specific sites on various targets is described in the literature. For example, these methods have been used to identify ligands that bind to specific sites on the targeting domain of tetanus neurotoxin (Cosman et al. (2002) Chem Res Toxicol 15: 1218-1228; Lightstone et al. (2000) Chem. Res. Toxicol., 13: 356-362) as well as eleven ligands we've already identified that bind to HLA-DR10.

Previous studies using a similar approach to identify ligands that bind to two sites on the targeting domain of tetanus neurotoxin (Id.) have required screening less than 30 ligands experimentally. Over half of the ligands predicted to bind to the protein were observed to bind experimentally. Thus, we believe that screening a set of 30 ligands per site should provide a sufficient number of compounds that bind to initiate SHAL synthesis. However, if in some embodiments, a suitable number of ligands (e.g., 3-5) are not identified to bind in the first round of NMR screens, additional sets of ligands may be selected and screened until suitable ligands are found that bind to two different sites in the target.

In certain embodiments, the selection of the ligand pairs (or other multi-ligand combinations) to be linked together is based on the following criteria (in descending order of importance): 1) binding site (the two ligands that comprise a pair preferably bind to different sites); 2) reliability of information obtained on available functional groups that may be used to attach the molecules to the linker without disrupting binding (analogs with known derivatives that are confirmed to bind, indicating the modification of particular functional group does not affect binding, are given priority); 3) the ligand's expected ease of attachment to the linker (the ligands preferably have functional groups that facilitate asymmetric attachment chemistry to put a different ligand on opposite ends of the linker); 4) relative binding affinity, e.g., as determined from the NMR diffusion experiments (ligands exhibiting the strongest binding to the protein are typically selected first); 5) known information on their toxicity in animals or humans (priority is typically given to use of ligands that are known to be non-toxic or have already been approved for use in other pharmaceuticals); and 6) ligand cost.

Because it is likely that a number of the individual ligands (binding moieties) may bind weakly to other proteins, it will not be necessary (or meaningful) to prescreen the individual ligands to determine if they bind to other proteins that might be encountered in the circulation before they are used to create SHALs. The binding of any single ligand (half of the pair used to create a bidentate SHAL) to other proteins is expected to be weak (micromolar at best). Consequently, the off-rate of the molecule will be high and the SHAL that only binds through one ligand to other proteins will be quickly removed from the tissue and circulation. High affinities (and low off-rates) typically will only be obtained when both ligands in the pair bind simultaneously to sites separated by the proper distance, which is dictated by the length of the linker connecting them. These two criteria will only be met when the SHAL comes in contact with the intended target. Once bound, the off-rate is expected to be reduced 1000 to 1,000,000-fold (based on previous studies) over that observed for either ligand alone. For this reason, cross-reactivity is not expected to be a significant complication.

E) Combined Computational/Empirical Approaches.

The binding of a SHAL to the region(s) of its target is based upon fit and charge and is dependent on the 3D structure and constituents of the binding moieties. In the computational approach described above, generating SHALs may involve definition/identification of attractive region(s) on the target molecule. Thus, for example, proteins of all types, including antigens, receptors and signaling proteins, may be modeled to find docking sites and ligands. The ligands may subsequently be tested using empiric methods. These methods typically require knowledge of the constituent molecules to be included in the modeling.

The empirical methods described above, rely on screening of libraries (e.g., combinatorial libraries) of potential ligands to find suitable binders. In this approach, no foreknowledge is required beyond availability of a target (e.g., protein, cell, etc.) of interest. Libraries are experimentally culled for binders. This may be followed by competition with a molecule, such as an antibody, peptide or chemical (ligand) known to react with the molecule in the region chosen to be targeted. This approach permits the elimination of binding chemicals or peptides that are not of interest and definition of those that bind to the region of interest.

A third approach is intermediate in nature and uses foreknowledge of attractive target molecules and regions of these molecules for initial competitive screening and counter-screening. Thus, for example, initial targets or ligand-library constituents may be computationally predicated. The optimized target or target collection and/or optimized library may then be screened and counter-screened as described herein to identify optimal binders.

F) Bead-Based Library Screening.

One approach for screening for ligands that bind the target molecules involves producing a combinatorial library comprising a large number of potential ligands each attached to a different bead/solid support. The combinatorial library may be a “random” library, or may be synthesized to provide numbers of variant having, e.g., particular (e.g., optimized) core chemistry.

Combinatorial synthetic methods are well known and are used to rapidly make large “libraries” of distinct compounds. In various embodiments the starting material (e.g., an amino acid) is covalently anchored to solid support. This is followed by the stepwise addition of monomers (typically protected monomers) such as amino acids, nucleotides, small organic molecules, and the like. Millions of distinct molecules may be created by varying number of steps and number of reactants (e.g., in a split-mix synthesis approach), but typically each bead contains only one compound.

The compounds comprising the library may be screened while still bound to the beads. Colorimetric, fluorometric, radiographic methods or other methods may be used to visualize positive (binding) beads. These may be captured (e.g., with a pipette, with a metal bar if the beads are magnetic), and the compound may then be characterized.

Thus, for example, one may synthesize a library of peptide ligands that bind HLA-DR10 molecules. Peptide synthesis chemistry is well developed. However, to obtain peptide ligands that have a longer half-life in vivo, one might choose to produce a peptide ligand library where the peptides comprise D-amino acids. Such peptides are expected to be more resistant to proteolysis in vivo. Moreover, D-amino acids are generally considered non-toxic.

The Lym-1 epitope on HLA-DR10 is highly polar. Thus, in synthesizing the library of potential binders on may select polar D-amino acids for synthesis (e.g., Ser, Asp, etc.) Using Split/Mix synthesis (see, e.g., U.S. Pat. No. 5,574,656) a library of D-peptides bound to beads is created.

Then HRP-tagged HLA-DR10 is added to the bead mixture. The HRP color label is visualized and the positive beads are removed. The positive beads may then be tested against, e.g., HLA-DR10 positive cell lines. Beads that test positive in this assay may then be tested against, e.g., a tissue panel to ensure that binding is HLA-DR10 specific. The specific binders in this assay may then be characterized (e.g., sequenced using Edman degradation, mass spectrometry, etc.).

Alternatively, there are strategies for encoding the identity of each the compound during the synthesis of the library (see, e.g., U.S. Pat. Nos. 5,565,324; 5,723,598; 5,834,195; 6,060,596; 6,503,759; 6,507,945; 6,721,665; 6,714,875; and the like). Using such “tagging” strategies the identity of the positive binders may then readily be determined.

G) Linking the Ligands (Binding Moieties) to Produce a Polydentate SHAL.

Once two more ligands (binding moieties) are identified that bind to different sites on the target, the ligands are linked either directly or through a linker to produce a polydentate SHAL. Where only two ligands are joined the SHAL is bidentate. Where three ligands are joined the SHAL is tridentate, and so forth.

A number of chemistries for linking molecules directly or through a linker are well known to those of skill in the art. The specific chemistry employed for attaching the ligands (binding moieties) to each other to form a SHAL will depend on the chemical nature of the ligand(s) and the “interligand” spacing desired. Ligands typically contain a variety of functional groups e.g., carboxylic acid (COOH), free amine (—NH2) groups, that are available for reaction with a suitable functional group on a linker or on the other ligand to bind the ligand thereto.

Alternatively, the ligand(s) may be derivatized to expose or attach additional reactive functional groups. The derivatization may involve attachment of any of a number of linker molecules such as those available from Pierce Chemical Company, Rockford Ill.

A “linker”, as used herein, is a molecule that is used to join two or more ligands (binding moieties) to form a polydentate SHAL. The linker is typically chosen to be capable of forming covalent bonds to all of the ligands comprising the SHAL. Suitable linkers are well known to those of skill in the art and include, but are not limited to, straight or branched-chain carbon linkers, heterocyclic carbon linkers, amino acids, nucleic acids, dendrimers, synthetic polymers, peptide linkers, peptide and nucleic acid analogs, carbohydrates, polyethylene glycol and the like. Where one or more of the ligands comprising the SHAL are polypeptides, the linkers may be joined to the constituent amino acids through their side groups (e.g., through a disulfide linkage to cysteine) or through the alpha carbon amino or carboxyl groups of the terminal amino acids.

In certain embodiments, a bifunctional linker having one functional group reactive with a group on the first ligand and another group reactive with a functional group on a second ligand may be used to form the desired SHAL. Alternatively, derivatization may involve chemical treatment of the ligand(s), e.g., glycol cleavage of the sugar moiety of glycoprotein, carbohydrate or nucleic acid with periodate to generate free aldehyde groups. The free aldehyde groups may be reacted with free amine or hydrazine groups on a linker to bind the linker to the ligand (see, e.g., U.S. Pat. No. 4,671,958). Procedures for generation of free sulfhydryl groups on polypeptide, such as antibodies or antibody fragments, are also known (See U.S. Pat. No. 4,659,839).

In certain embodiments, lysine, glutamic acid, and polyethylene glycol (PEG) based linkers different length are used to couple the ligands. A number of SHALs have been synthesized using a combination of lysine and PEG to create the linkers. Chemistry of the conjugation of molecules to PEG is well known to those of skill in the art (see, e.g., Veronese (2001) Biomaterials, 22: 405-417; Zalipsky and Menon-Rudolph (1997) Pp. 318-341 In: Poly(ethyleneglycol) Chemistry and Biological Applications. J. M. Harris and X. Zalipsky (eds)., Am. Chem. Soc. Washington, D.C.; Delgado et al. (1992) Drug Carrier Syst., 9: 249-304; Pedley et al. (1994) Br. J. Cancer, 70: 1126-113-0; Eyre and Farver (1991) Pp. 377-390 In: Textbook of Clinical Oncology, Holleb et al. (eds), Am. Cancer Soc., Atlanta Ga.; Lee et al. (1999) Bioconjug. Chem., 10: 973-981; Nucci et al. (1991) Adv. Drug Deliv., 6: 133-151; Francis et al. (1996) J. Drug Targeting, 3: 321-340).

One advantageous feature of the synthetic scheme used to create these SHALs is that the approach allows the attachment of almost any type of molecule to a third site on the linker. In the first round of SHAL synthesis, biotin has been attached at this site to facilitate the in vitro binding studies. The biotin tag makes it possible to quickly measure the binding to the isolated protein by surface plasmon resonance and examine the selectivity of the SHAL for binding to live cells and tissue sections.

Once the SHAL has been tested and confirmed to bind to the target (e.g., HLA-DR10), metal chelators such as DotA (or other effectors) may be attached in the final round of synthesis to enable the delivery of radionuclides or other effectors to target bearing cells (e.g., tumor cells).

After retesting the effector-SHAL conjugates to reconfirm their ability to bind to the target, the conjugates exhibiting the best selectivity for their targets can, optionally be tested for their biodistribution in test organisms (e.g., mice). Other unique molecules may also be attached to this site in future studies so these same SHALs may also be used, for example, to test the utility of pre-targeting approaches for radioisotope delivery.

H) Stepwise Solid-Phase SHAL Synthesis.

In certain embodiments, SHAL synthesis proceeds by a stepwise-solid phase synthesis approach. In this approach each linker component or ligand is attached onto a growing molecule (SHAL) covalently attached to the surface of a resin. After each chemical reaction the resin may be extensively washed to remove the unreacted products.

In one approach, DotA was attached to the linker at the beginning of the synthesis. After the excess DotA was washed away, multiple additional chemical reactions that were carried out on the resin to add the various linkers and ligands, and after each reaction the unreacted products were again washed away. By the time the synthesis of the SHAL was completed, the amount of free DotA present in the sample was undetectable when examined by HPLC and mass spectroscopy. The DotA link is extremely stable, so it does not come off the SHAL once it's been attached.

I) Screening SHALs for Affinity and Selectivity.

In certain embodiments, a library of SHALS comprising different ligands (binding moieties) and/or comprising different length linkers is screened to identify those SHALS that have the best affinity and/or selectivity for the target. Such screening assays may be performed in a number of formats including, but not limited to screening for binding to isolated targets, screening for binding to cells in culture, screening for binding to cells in tissue arrays, and screening for in vivo binding to the desired target.

1. SHAL Binding to Isolated Targets (e.g., Proteins).

In certain embodiments, the binding affinities of the best SHALs may be estimated by mass spectrometry of the SHAL-target complexes, followed by a more accurate surface plasmon resonance (SPR) spectroscopy (Shuck (1997) Annu Rev Biophys Biomol Struct., 26: 541-566; Van Regenmortal (2001) Cell Mol Life Sci., 58: 794-800) measurement of the SHAL-target binding affinity using for example, the IASYS Plus or BiaCore instruments. In order to perform the SPR measurement, biotin may be added to the linker through a third functional group (as described above) and the SHAL may be bound to commercially available streptavidin coated chips. In certain preferred embodiments, only those SHALs exhibiting nM or higher binding affinities may be considered useful. The SHALs exhibiting the greatest affinity may then be tested for their selectivity. Experiments may be performed to test the selectivity of SHAL binding to targets in the presence of molecules related to the targets. Thus, for example, where the SHAL is directed to HLA-DR10, the SHAL may be evaluated for its ability to bind target molecules in the presence of Raji cell surface proteins extracted and separated by affinity chromatography. After treating the gel with the biotinylated SHAL and rinsing out excess unbound SHAL, the location of the bound SHAL may be detected by staining with Rhodamine tagged streptavidin. In certain embodiments, the SHALs that are considered to exhibit reasonable protein selectivity may be those molecules in which 95% or more of the fluorescence is associated with the HLA-DR10 monomer and multimer peaks.

2. SHAL Binding to Cells in Culture.

Where the SHAL target is a marker on a cell (e.g., macrophages, B Cells, Dendritic Cells) it may be desired to assess the specificity of binding of the SHAL to intact cells.

Cell binding studies may be conducted with the biotinylated (or otherwise labeled) SHALs, using for example the fluorescence of bound Rhodamine-tagged streptavidin to confirm the SHALs bind to target (e.g., Raji) cells. If the SHAL is observed to bind, SPR measurements may be conducted to determine the affinity of intact cells to the SHAL. In certain embodiments, those SHALs that exhibit at least a 2-fold, preferably at least a 5-fold, and more preferably at least a 10-fold difference in the staining intensity of target (e.g., tumor) cells over controls may be selected for further testing and development. Analogs of the most promising SHALs may be synthesized with a DotA molecule attached to the linker, and binding experiments may be conducted using radionuclide-tagged SHALs to obtain more quantitative data and also attempt to determine if the SHAL is retained on the surface of the cell or is internalized using NanoSIMS or other methods. This information is useful in making decisions about the type of radioisotope that is to be loaded into the chelator. If the SHAL remains on the surface, the SHAL is typically utilized alone or with effectors that do not require internalization (e.g., alpha emitters such as ⁹⁰Yttrium, various detectable labels, and the like). If evidence is obtained to suggest the SHAL is internalized upon binding to the target cells, it is possible to utilize the SHAL with effectors that are active when internalized.

3. Analysis of Cell Selectivity Using Tissue Arrays.

Tissue array technology may be used to screen SHALs to determine tissue specificity (e.g., malignant and normal tissue reactivity in the case of anti-tumor SHALs). Tissue arrays are well known to those of skill in the art (see, e.g., Kononen et al. (1998) Nat. Med., 4:844-847; Torhorst et al. (2001) Am J Pathol., 159: 2249-2256; Nocito et al. (2001) Int J Cancer, 94: 1-5, and the like). In its basic form, a tissue microarray is formed by taking small cores of each individual tumor case/block and assembling these cores into a single block (Id.). By sectioning this new block, standard immunohistochemistry and in-situ hybridization techniques may be used. Therefore, one may assay hundreds of tissue samples in one experiment rather than having to perform hundreds of different experiments. FIGS. 2 and 3 outline how the tissue arrays may be used and show a diagram of illustrative tissue array.

In one embodiment, for the normal tissue array we have identified 80 unique tissues, which include oropharynx, heart, lung, stomach, spleen, liver, kidney, intestine, bone marrow, pancreas, bladder, muscle, adrenal, breast, brain, normal prostate and skin for placement on the tissue microarray. For a lymphocyte specific tissue array, we have included neoplastic lymphocytic lines, xenografts, and patient material collected from the Human Biological Specimen Repository at UC Davis. Using these or similar tissue arrays, one may determine the non-specific binding of the SHALs to normal tissue and specific binding to lymphocytic and prostatic neoplasms. The hybridization of the SHALs to the tissue arrays is straightforward. Using biotinylated SHALS as described herein, labeled streptavidin (e.g., Rhodamine-tagged streptavidin) may readily be used to identify those cells that bind the SHALs. When required, the tissue microarray results may be verified by conventional histology and immunohistology.

In one illustrative approach, 2-4 tissue cylinders, with a diameter of 0.6-mm, may be punched from the selected areas of each “donor” tissue block and brought into a recipient paraffin block in order to assemble the tissue microarray, using a Tissue Microarrayer (e.g., Beecher Instruments, Silver Spring, Md.) and the techniques described by Kononen et al. (1998) Nat. Med., 4:844-847. Tissue microarray slides containing, for example, 200-400 cores may then be sectioned at a thickness of 4 Routine Hematoxylin & Eosin staining may be performed in order to verify that each core represents its selected histopathology. For immunohistochemistry, microwave in a citrate buffer may be used for antigen retrieval. The images of the slides may be captured by confocal scanner (ScanScope, Mountain View, Calif.) and visualized with MrSid Viewer 2.0 (LizardTech, Inc.) as described below. The ability to view the tissue array images on a computer rather than a microscope dramatically increases the efficiency of analysis.

The major obstacle to digital pathology has been the representation of glass slides in a digital format. Unlike radiology, which begins with a digital representation of a patient rendered by CT, MRI, or now “digital plain film”, pathology requires that all tissue samples be processed and made into stained tissue sections mounted on glass slides for interpretation. The new technology produces images of the entire glass slide, thereby producing a true digital representation of the entire histopathologic specimen (Whole Slide Imaging). Most of the current instruments use a microscope equipped with a digital camera and a robotic stage to capture thousands of individual images. Each image is focused by the content expert. Once acquired, these images are typically be stitched (or tiled) together to form the final representation of the slide. This process is both very time consuming and, due to the high number of images involved, the images are often misaligned.

ScanScope, (Aperio® Technology) is a new type of digital slide scanner that scans a microscope slide in 3 to 5 minutes, capturing 8 to 12 gigabyte images at 50,000 dpi. The images are then compressed, processed and stored for presentation (see below). MrSid® by Lizardtech® compresses the large, 8 to 12 gigabyte images using a proprietary multi-layer wavelet Jpeg format with compression ratios reaching 20:1 without significant image degradation. The images may then be either viewed locally or served from a web server. Unlike standard web-based still images, which are typically downloaded to be viewed within a browser, the MrSid processed images are viewed from the web, and the browser application never downloads the entire image. Because the images are acquired at their maximal resolution, “lower” magnification views of an image are constructed by the server. Using the combination of ScanScope and Zoomify browser, entire slides (12 gigabytes) may be captured and processed in less than 20 minutes.

J) Optimization of SHAL Affinity, Selectivity and Metabolism by Varying the Linker Length and Linker and Ligand Structure.

SHAL affinity, selectivity and metabolism may be optimized by varying the linker length, and/or the linker and ligand structure, using computer modeling and experimental studies. Linker lengths may be reduced or increased to improve the SHAL's affinity for its target. Changes in the individual ligands used to create the SHAL or alterations in individual ligand structure may also be made to improve binding, target selectivity and optimize the clearance of unbound SHAL from the organism. Modifications in the structure of the linker itself may also be considered to facilitate SHAL clearance, if necessary, from normal tissues and peripheral blood through the incorporation of cleavable bonds (e.g., a peptide or other cleavable linker) that attach the chelator to the SHAL.

If a particular SHAL is observed to exhibit non-specific binding (e.g., to many proteins in the cell extracts or to both Raji and control cells), additional SHALs may be synthesized using different pairs of ligands until a suitably specific SHAL is identified.

1. Maximization of SHAL Binding Affinity for Target Molecule(s).

Binding affinity of multidentate reagents to protein or cell surface targets may be increased by one to several orders of magnitude by changing and optimizing the length of the linker separating the ligands. Without being bound to a particular theory, it is believed that this increase is related to achieving the optimal separation between the ligands to allow them to bind to their individual sites as well as to providing sufficient rotational flexibility within the linker itself to enable the optimal interaction of each ligand within its binding site (e.g., binding pocket).

In certain embodiments, the initial linker length that is chosen for use in the initial SHALs is identified by estimating the distance between the two (or more) bound ligands that are to be linked together. Once it has been determined that a particular combination of linked ligands actually binds to the target, additional modeling may be conducted to further refine the length of the linker and optimize the SHALs binding affinity.

For example, where the target is HLA-DR10, the structure of the HLA-DR10 beta subunit may modeled with both ligands bound in their respective pockets and various length PEG linkers interconnecting the ligands (see, e.g., the Examples herein). From molecular dynamics studies the orientations of the bound ligands may be evaluated to improve the linker design. Further molecular dynamics simulations may be performed to include the linkers and the ligands, thus simulating the polydentate ligands interacting with the target, e.g., as described herein.

Once the results of these modeling experiments are obtained, an additional set of SHALs may be synthesized with linkers spanning the range of sizes predicted to be optimal, and their binding affinities may be experimentally tested.

2. Optimization of Target Selectivity and Metabolism of SHAL.

Computational methods may also be sued to determine if changes in the structure of the individual ligands that are linked together to produce the SHAL improve target selectivity and optimize SHAL metabolism and its clearance from normal tissues and peripheral circulation. This may be accomplished, for example, by examining the types of functional groups present inside a targeted binding pocket and their location relative to the bound ligand.

Molecular dynamics studies may be conducted using different conformations of the ligand and selected ligand analogs to aid the identification of ligand derivatives that fit optimally into each binding site (e.g., pocket). Diffusion NMR experiments (Lin et al. (1997) J. Organic Chem., 62: 8930-8931) may be conducted to compare and rank the affinities of a subset of the ligand analogs. The particular analogs chosen for analysis are typically selected based on the results provided by computer modeling and the analog's commercial availability or ease of synthesis. If higher affinity analogs are identified experimentally, a set of new SHALs may be synthesized and tested for their affinity, selectivity in binding to targets, and desirable metabolic properties (e.g., rapid clearance from peripheral circulation, liver and kidney).

In certain embodiments, the small size of the SHAL may result in its being cleared from the tissues too quickly to be effective in delivering a suitable amount of effector to the target cells. If this is observed, various approaches may be used to optimize the retention time of the SHAL in the target tissue. One involves using a biotin attachment site on the linker to add a third ligand that binds to another site on the target. This is expected to increase the affinity of the SHAL to subpicomolar levels and reduce the off-rate of the bound molecule dramatically. Alternatively, the effective size of the SHAL may be increased substantially by attaching it to larger, multi-arm PEG molecules and/or to other molecules.

K) Illustrative SHALs.

Using the teachings provided herein, a number of different SHALs that bind, for example targets (e.g., HLA-DR10) may readily be prepared. In various embodiments the SHALS include, but are not limited to bidentate SHALS (comprising two binding ligands), tridentate SHALS (comprising three binding ligands), tetradenate SHALS (comprising four binding ligands), pentadentate SHALS (comprising five binding ligands), and so forth. In various embodiments may be multimeric (e.g., structures and/or complexes comprising two, three, four, five, or more SHALs). The SHALs may be homomultimeric (comprising two, three, four, five, or more of the same type of SHAL), or heteromultimeric (comprising, for example, two, three, four, or five or more SHALS where at least two are different species of SHAL).

In certain embodiments the SHALS comprise one or more, preferably two or more, or three or more ligands described in any of Tables 1, 5, 6, 7, or 8 and/or analogues thereof. Certain preferred SHALS include, but are not limited to bidentate SHALS, tridentate, dimeric bidentate SHALS, bimeric (bis) tridentate SHALS, and the like. In certain embodiments, the SHALS comprise one or more of the ligands shown in Table 1.

TABLE 1
Illustrative ligands that may be included in SHALS as
described herein. Some of these (marked with *) may be
used in place of the Ct ligand or attached to the linker as a fourth
component, functioning not to help the SHAL bind to the protein better
but as an inhibitor once the SHAL gets inside the cell.

| | |
|---|---|
| 1 | BOC-4-aminomethyl-L-Phe |
| 2 | *4[[5-(Trifluoromethyl)pyridin-2-yl]oxy]phenyl]N-phenylcarbamate |
| 3 | *(R)-2-[4-(5-chloro-3-fluoro-2-pyridyloxy)phenoxy]propionic acid |
| 4 | *2-(((3-chloro-5(trifluoromethyl)pyridin-2- |
| | yloxy)phenoxy)methyl)acrylates |
| 5 | *2(((3-chloro-5(trifluoromethyl)pyridin-2- |
| | yloxy)phenyl)methyl)acrylates |
| 6 | *2-(((3-chloro-5(trifluoromethyl)pyridin-2- |
| | yloxy)phenyl)methyl)acrylonitriles |
| 7 | *3-(3-chloro-4-{[5-(trifluoromethyl)-2-pyridinyl]oxy}anilino)-3- |
| | oxopropanoic acid |
| 8 | *Sethoxydim |
| 9 | *Clethodim |
| 10 | *5-(Tetradecyloxy)-2-furoic acid |
| 11 | *2-[(2,6-Dichlorophenyl)amino]benzeneacetic acid |
| 12 | *2-[4-(4-Chlorophenoxy)phenoxy]propanoic acid |
| 13 | *(RS)-2-{4-[3-chloro-5-(trifluoromethyl)-2- |
| | pyridinyl]oxy]phenoxy}propanoic acid |
| 14 | *(RS)-2-[4-(6-chloro-1,3-benzoxazol-2-yloxy)phenoxy]propanoic acid |
| 15 | *(RS)-2-[4-(2,4-dichlorophenoxy)phenoxy]propanoic acid |
| 16 | *(RS)-2-{4-[5-(trifluoromethyl)-2-pyridyloxy]phenoxy}propanoic acid |
| 17 | *(RS)-2-[4-(6-chloroquinoxalin-2-yloxy)phenoxy]propanoic acid |
| 18 | *(RS)-2-[4-(α,α,α-trifluoro-p-tolyloxy)phenoxy]propanoic acid |
| 19 | 5-([4,6-Dichlorotriazin-2-yl]amino)fluorescein hydrochloride |
| 20 | 3-[N-(4-acetylphenyl)carbomoyl]pyridine-2-Carboxylic acid |
| 21 | 3-(2-{[3-chloro-5-(trifluoromethyl)-2-pyridinyl]oxy}anilino)-3- |
| | oxopropanoic acid |
| 22 | L-ornithine-beta-alanine |
| 23 | 2-Methyl-1-(3-morpholinopropyl)-5-phenyl-1H-pyrrole-3- |
| | carboxylic acid |
| 24 | Hippuric acid |
| 25 | Hippuryl-D-lysine |
| 26 | Hippuryl-L-phenylalanine |

TABLE 2
List of illustrative multidentate and dimeric, multidentate SHALs and their molecular weight
(MW in Daltons). DotA on the SHAL contributes an additional 400 (or 244 for biotin)
Daltons.

| Acronym | Identity | MW |
|---|---|---|
| Multidentate | | |
| LeacPLD | acetylated 5-leuenkephalin PEG lysine | 1,505 |
| | deoxycholate | |
| ItPLD | triiodothyronine PEG lysine deoxycholate | 1,559 |
| DvLPBaPL | dabsyl-L-valine lysine PEG N-benzoyl-L-arginyl- | 1,317 |
| | 4-amino-benzoic acid PEG lysine | |
| CtLPTPL | 3-(2-([3-chloro-5-trifluoromethyl)-2- | 1,163 |
| | pyridinyl]oxy)-anilino)-3-oxopropanionic acid | |
| | lysine PEG L-thyronine PEG lysine | |
| CtLPBaPL | 3-(2-([3-chloro-5-trifluoromethyl)-2- | 1,287 |
| | pyridinyl]oxy)- | |
| | anilino)-3-oxopropanionic acid lysine PEG N- | |
| | benzoyl-L-arginyl-4-amino-benzoic acid PEG | |
| | lysine | |
| DvPLLCtPCbL | dabsyl-L-valine PEG lysine lysine 3-(2-([3-chloro- | 1,765 |
| | 5-trifluoromethyl)-2-pyridinyl]oxy)-anilino)-3- | |
| | oxopropanionic acid PEG 4-[4-(4- | |
| | Chlorobenzyl)piperazinol-3- | |
| | nitrobenzenecarboxylic acid lysine | |
| Dimeric, Multidentate | | |
| (LeacPLD)₂LP | (acetylated 5-leuenkephalin PEG lysine | 3,006 |
| | deoxycholate)₂lysine PEG | |
| (ItPDP)₂LL | (triiodothyronine PEG deoxycholate PEG)₂lysine | 3,113 |
| | lysine | |
| (DyLPBaP)₂LL | (dabsyl-L-valine lysine PEG N-benzoyl-L-arginyl- | 2,626 |
| | 4-aminobenzoic acid PEG)₂lysine lysine | |
| (DvLPBaPPP)₂LL | (dabsyl-L-valine lysine PEG N-benzoyl-L-arginyl- | 3,210 |
| | 4- | |
| | aminobenzoic acid PEG PEG PEG)₂lysine lysine | |
| (DvLPBaPPPP)₂LL | (dabsyl-L-valine lysine PEG N-benzoyl-L-arginyl- | 3,501 |
| | 4-aminobenzoic acid PEG PEG PEG PEG)₂ lysine | |
| | lysine | |
| (DvLCsPBaPPP)₂CsLL | (dabsyl-L-valine lysine cysteic acid PEG N- | 3,666 |
| | benzoyl-L-arginyl-4-aminobenzoic acid PEG PEG | |
| | PEG)₂ cysteic acid lysine lysine | |
| (DvPLLCtPCbPPP)₂LL | (dabsyl-L-valine PEG lysine lysine 3-(2-([3- | 4,267 |
| | chloro-5-trifluoromethyl)-2-pyridinyl]oxy)- | |
| | anilino)-3-oxopropanionic acid PEG 4-[4-(4- | |
| | chlorobenzyl)piperazino]-3- | |
| | nitrobenzenecarboxylic acid PEG PEG | |
| | PEG)₂ lysine lysine | |

L) SHALS Attached to Transduction Peptides.

In certain embodiments the SHALs include (e.g., are attached to) one or more transduction peptides. A transduction peptide is a peptide that acts as a “transmembrane shuttle” facilitating entry of the peptide into a cell (e.g., facilitating penetration of the cell membrane). Transduction peptides are well known to those of skill in the art. Such peptides include, but are not limited to the nuclear localization signal (NLS) of simian virus 40 (SV40) T antigen ((Yoneda (1997) J. Biochem., 121: 811-817), the protein transduction domain of HIV Tat protein (Tat peptide) (Vives et al. (1997) J. Biol. Chem. 272: 16010-16017; Schwarze et al. (1999) Science 285: 1569-1572; Torchilin et al. (2003) Proc. Natl. Acad. Sci., USA, 100: 1972-1977.), the integrin-binding peptide (RGD peptide) (Hart et al. (1994) J. Biol. Chem. 269: 12468-12474), the heparin-binding domain of vitronectin (VN peptide) (Vogel et al. (1993) J. Cell Biol. 121: 461-4-68), antennapedia protein of Drosophila (see, e.g., Joliot et al. (1991) Proc. Natl. Acad. Sci., USA, 88: 1864-1868), penetratin (Tseng et al. (2002) Mol. Pharmacol., 62: 864-887), intact proteins that naturally pass through cell membranes (the herpes virus protein VP22 (Phelan et al. (1998) Nat. Biotechnol., 16: 440-443), synthetic cationic peptide transporters such as oligoarginine (Tung and Weissleder (2003) Adv. Drug Delivery Rev., 55: 281-294; Futaki (2005) Adv. Drug Delivery Rev., 57: 547-558), lactosylated poly-L-lysine (Midoux et al. (1993) Nucl Acids Res., 21: 871-878), short peptide sequences selected from phage display libraries (Kamada et al. (2007) Biol Pharm Bull. 30: 218-223; see also peptides 1-6 in Table 3) that exhibit sequence similarities to know peptide shuttles, and the like.

TABLE 3
Amino acid sequences of illustrative
transduction peptides.

| | | Seq |
|---|---|---|
| | | ID |
| Peptide | Sequence | No. |
| 1 | S-G-E-H-T-N-G-P-S-K-T-S-V-R-W-V- | 05 |
| | W-D | |
| 2 | S-M-T-T-M-E-F-G-H-S-M-I-T-P-Y-K- | 06 |
| | I-D | |
| 3 | Q-D-G-G-T-W-H-L-V-A-Y-C-A-K-S-H- | 07 |
| | R-Y | |
| 4 | M-S-D-P-N-M-N-P-G-T-L-G-S-S-H-I- | 08 |
| | L-W | |
| 5 | S-P-G-N-Q-S-T-G-V-I-G-T-P-S-F-S- | 09 |
| | N-H | |
| 6 | S-S-G-A-N-Y-F-F-N-A-I-Y-D-F-L-S- | 10 |
| | N-F | |
| 8 | G-T-S-R-A-N-S-Y-D-N-L-L-S-E-T-L- | 11 |
| | T-Q | |
| Tat13 | G-R-K-K-R-R-Q-R-R-R-P-P-Q | 12 |
| Antermapedia | R-Q-I-K-I-WF-Q-N-R-R-M-K-WK-K | 13 |
| VP22 | N-A-K-T-R-R-H-E-R-R-R-K-L-A-I-E- | 14 |
| | R | |
| hexa-Arg | R-R-R-R-R-R | 15 |

The foregoing list of transduction peptides is intended to be illustrative and not limiting. Other transduction peptides will be known to and readily available to one of ordinary skill in the art and using the teaching provided herein may readily be incorporated into/attached to a SHAL. A review of illustrative transduction peptides is provided by Derossi et al. (1998) Trends Cell Biol. 8: 84-87.

### 4) Ligands.

In various embodiments the effector molecule may also be a ligand, an epitope tag, or an antibody. Particularly preferred ligand and antibodies are those that bind to surface markers on immune cells.

In certain embodiments, the effector comprises a viral particle. The SHAL may be conjugated to the viral particle e.g., via a protein expressed on the surface of the viral particle (e.g., a filamentous phage).

B) Attachment of the SHAL to the Effector.

One of skill will appreciate that the SHALs of this invention and the effector molecule(s) may be joined together in any order. Thus, in various embodiments, the effector may be attached to any ligand comprising the SHAL and/or to the linker joining the various ligands comprising the SHAL.

The SHAL and the effector may be attached by any of a number of means well known to those of skill in the art. Typically the effector is conjugated, either directly or through a linker (spacer), to the SHAL.

In one embodiment, the SHAL is chemically conjugated to the effector molecule (e.g., a cytotoxin, a label, a ligand, or a drug or liposome, etc.). Means of chemically conjugating molecules are well known to those of skill.

The procedure for attaching an effector to a SHAL will vary according to the chemical structure of the effector and/or the SHAL. The ligands comprising the SHAL and/or the linker joining the ligands may contain a variety of functional groups; e.g., carboxylic acid (COOH), free amine (—NH₂), hydroxyl (—OH), thiol (—SH), and other groups, that are available for reaction with a suitable functional group on an effector molecule or on a linker attached to an effector molecule to effectively bind the effector to the SHAL.

Alternatively, the ligand(s) comprising the SHAL and/or the linker joining the ligands may be derivatized to expose or attach additional reactive functional groups. The derivatization may involve attachment of any of a number of linker molecules such as those described above for coupling the ligands to each other.

In some circumstances, it is desirable to free the effector from the SHAL when the chimeric molecule has reached its target site. Therefore, chimeric conjugates comprising linkages that are cleavable, e.g., in the vicinity of the target site may be used when the effector is to be released from the SHAL. Cleaving of the linkage to release the agent from the antibody may be prompted by enzymatic activity or conditions to which the conjugate is subjected, e.g., either inside the target cell or in the vicinity of the target site. When the target site is a tumor, a linker which is cleavable under conditions present at the tumor site (e.g., when exposed to tumor-associated enzymes or acidic pH) may be used.

In certain instances, the cleavable linker may be a peptide that may be subject to proteolysis. In certain embodiments, the cleavable linker comprises a peptide having a recognition site for a protease.

A number of different cleavable linkers are known to those of skill in the art. See U.S. Pat. Nos. 4,618,492; 4,542,225, and 4,625,014. The mechanisms for release of an agent from these linker groups include, for example, irradiation of a photolabile bond and acid-catalyzed hydrolysis. U.S. Pat. No. 4,671,958, for example, includes a description of immunoconjugates comprising linkers which are cleaved at the target site in vivo by the proteolytic enzymes of the patient=s complement system. In view of the large number of methods that have been reported for attaching a variety of radiodiagnostic compounds, radiotherapeutic compounds, drugs, toxins, and other agents to antibodies one skilled in the art will be able to determine a suitable method for attaching a given agent to an antibody or other polypeptide.

### EXAMPLES

The following examples are offered to illustrate, but not to limit the claimed invention.

### Example 1 Creation of HLA-DR10 Specific SHALs

Preclinical and clinical studies have revealed that the epitopic region (unique region recognized by antibodies) on the beta subunit of HLA-DR 10, and related HLA-DR major histocompatibility cell surface proteins, are particularly attractive targets for systemic radioisotopic therapy for B-cell lymphomas and leukemias and provide other opportunities for cancer treatment and prevention. Although HLA-DR 10 has characteristics in common with other B-cell surface proteins, like CD20, that make it a suitable target, it has disparate characteristics that we believe provide great attractiveness.

In common with CD20 antigen, the HLA-DR 10 protein is located on the surface of B lymphocytes, persists through B-cell differentiation, but disappears during transformation of the lymphocyte to the plasma cell stage (Epstein et al. (1987) Cancer Res., 47: 830-840). The discrete Lym-1 antigen epitope appears on committed B-cell precursors, but is not expressed earlier during B-cell development. In addition, it is not generally found on T cells or other normal cells. Expression of class 2 MHC molecules on B-cells is developmentally controlled. Early and pre-B-cells are class 2 mRNA negative and cannot be induced to express class 2 antigens. HLA-DR antigen is acquired during the late pre-B-cell stage. Because the basal level of class 2 expression on B-cells is about 100 times lower than that found on malignant B-cell lines (Rose et al. (1996) Cancer Immunol Immunother., 43: 26-30; Rose et al. (1999) Mol. Immunol., 36: 789-797), this provides an explanation for the observation that only 5 mg of Lym-1 antibody is needed to target extravascular malignant lymphoma (DeNardo et al. (1998) J Clin Oncol., 16: 3246-3256), whereas 50 mg of CD22 and hundreds of mg of CD20 antibodies are required due to the density of CD22 and CD20 antigens on normal lymphocytes (Press (1999) Semin Oncol., 26: 58-65; Knox et al. (1996) Clin Cancer Res., 2: 457-470). Treatment doses of iodine-131, copper-67, or yttrium-90 attached to small amounts of Lym-1 cures most mice with Raji xenografts (DeNardo et al. (1991) Antibody Immunoconj Radiophar., 4: 777-785; DeNardo et al. (1997) Clin Cancer Res., 3:71-79), the human Burkitt's malignant lymphoma cell line used as the immunogen to generate Lym-1 (Epstein et al. (1987) Cancer Res., 47: 830-840). Similarly, these radiopharmaceuticals, in Phase I/II trials in patients with B-cell non-Hodgkin's lymphoma and a subset of patients with chronic lymphocytic leukemia, have induced a high and durable response rate, with frequent complete remissions and some long-term survivals when used as single agent therapy. HLA-DR provides cell identification, and antigenic peptides are displayed on HLA-DR. This may explain unusually long survivals in a subset of the patients with aggressive lymphoma in whom an idiotypic antibody cascade, including human polyclonal antibodies cytotoxic for Raji cells and Raji tumors, has been documented (DeNardo et al. (1998) Cancer Biother Radiopharm., 13: 1-12; Lamborn et al. (1997) Clin Cancer Res., 3: 1253-1260).

However, as well as these antibodies work, there is still a need to improve upon them. The antibody is a macromolecule that penetrates vascular barriers and the tumor poorly and interacts with a variety or receptors, which limits their selectivity as radioisotope carriers and adds to the adverse event profile. The immunogenicity of antibody-based reagents may be minimized, but not eliminated, using “humanized” antibodies (Brown et al. (2001) Clin Lymphoma., 2: 188-90; Kostelny et al. (2001) Int J Cancer, 93: 556-65; Leonard et al. (2002) Semin Oncol., 29: 81-6; Lundin et al. (2002) Blood, 100: 768-73; Ligibel and Winer (2002) Semin Oncol., 29: 38-43). Immunogenicity may be avoided by creating non-protein based reagents. Whole antibodies also exhibit appreciable reactivity (e.g., Fc interactions) with non-target cells that reduces selectivity and increases adverse events. Even small improvements in the targeting agent's selectivity may be used to minimize collateral damage and enhance the drug's therapeutic index.

Lym-1 is a murine IgG-2a monoclonal antibody (MAb) that selectively binds a protein highly expressed on the surface of malignant human B-cells (Epstein et al. (1987) Cancer Res., 47: 830-840). We have shown that a discrete epitope on HLA-DR 10 was the original antigen in Raji cells that generated the Lym-1 MAb, and this epitope is not shared by all HLA-DR subtypes (Rose et al. (1996) Cancer Immunol Immunother., 43: 26-30; Rose et al. (1999) Mol. Immunol., 36: 789-797). Data suggest that the critical Lym-1 binding residues are contained in the 19 differences in amino acid sequence between the reactive HLA-DR 10 beta subunit and the unreactive, largely identical HLA-DR 3 and HLA-DR 52 beta subunits. This serves as the basis for the selectivity of the Lym-1 epitope or binding site among HLA-DR containing white blood cells, yet provides the basis for the existence of this protein in virtually all patients with malignant B-cells. Of the 19 residues comprising the critical Lym-1 binding region, only the amino acids Q70 or R70, followed by R71 were found in all Lym-1 reactive specimens and were absent in Lym-1 unreactive specimens. In many of the unreactive HLA-DR molecules, these two residues were often replaced by D70 and/or E71. The hypothesis that the subtypes containing the putative critical Lym-1 binding residues (Q/R70-R71) would be most reactive has been confirmed in a series of studies including extensive cytotoxicity assays conducted in lymphoblastoid cell lines of B and T cell type, incorporating 31 HLA-DR genotypes (Rose et al. (1999) Mol. Immunol., 36: 789-797). All the strongly reactive cells expressed at least one Q/R70-R71-containing HLA-DR allele while none of the least reactive cell lines expressed that sequence at position 70-71 of the beta chain. Cytotoxicity assays also showed that the former were dramatically more affected than the latter (Id.). Although Lym-1 reacted with peripheral blood lymphocytes from healthy donors, the avidities were much lower, consistent with a lower HLA-DR protein density on normal lymphocytes and the hypothesis that univalent rather than bivalent binding may occur, further explaining the selectivity of Lym-1 for malignant cells in patients with lymphoma (Id.). Thus, it seems that both the critical Lym-1 glutamine/arginine residues and a threshold antigen density contribute to the selectivity of Lym-1 binding to malignant B-cells over normal lymphocytes. In any event, the data confirm that Lym-1 binds preferentially to lymphoblastoid cells over normal PBLs, thereby providing an attractive difference from other malignant B-cell targeting proteins (Id.).

The experience with CMRIT has led us to appreciate the complexities of implementation and to realize that many patients with advanced NHL are ineligible for BMT because of their disease and insufficient marrow harvest. For these reasons and because of unique opportunities to dose intensify using novel approaches to develop targeting molecules that may dramatically improve the therapeutic index, we have developed high affinity ligands (SHALs) that mimic ¹³¹I-iodide in thyroid cancer. ¹³¹I-iodide in thyroid cancer, the prototype for radioisotopic molecular targeted systemic radiotherapy, has led to cure of otherwise incurable thyroid cancer because the ¹³¹I is rapidly trapped and retained by the cancer or excreted in the urine, providing a therapeutic index approaching infinity and the opportunity to administer almost unlimited radioisotope without significant toxicity.

We believe that small molecule SHALs may better fulfill the potential of “RIT”, and represent a natural extension of these ongoing translational activities involving HLA-DR as a target for radioisotopic carrier molecules to deliver systemic radiotherapy. As described below, we have synthesized a number of bidentate SHALs and determined that at least one of these SHALs binds to isolated HLA-DR10.

A) Development of a Computer Model of the Molecular Structure of the HLA-DR 10 Beta Subunit Containing the Region Shown to be Critical for Lym-1 Antibody Binding to Malignant B Cells and Compare the Structure with Other HLA-DR Molecules.

Crystal structures for four different closely related HLA-DR molecules (HLA-DR 1-4) have been determined previously and deposited in the PDB structure database by others (Jardetzky et al. (1994) Nature, 368:711-718; Bolin et al. (2000) J. Med. Chem., 43:2135-2148; Smith et al. (1998) J. Exp. Med., 188:1511-1520; Ghosh et al. (1995) Nature, 378:457-462). Protein sequences for these four proteins, HLA-DR1, HLA-DR2, HLA-DR3 and HLA-DR4, were aligned with the HLA-DR10 sequence and compared to identify both the locations of the variable amino acids and those regions of the HLA-DR10 molecule containing the amino acid residues that had been identified as the critical epitope of the Lym-1 antibody (Rose et al. (1996) Cancer Immunol Immunother., 43: 26-30; Rose et al. (1999) Mol. Immunol., 36: 789-797). This alignment revealed that all five proteins exhibit such a high degree of sequence similarity that we were able to create a sufficiently accurate 3-D model of the HLA-DR10 beta subunit by homology modeling and use the coordinates of the model to screen for ligand binding using the program DOCK.

Two different approaches were used to create models of the HLA-DR10 beta subunit for use in ligand docking. The first approach used the coordinates of the entire structure of HLA-DR3 as the template for creating the homology model, and the nineteen amino acids that differed between HLA-DR3 and HLA-DR10 were mutated (changed) in the HLA-DR3 sequence. The coordinates of the amino-terminal four amino acids, which are present in HLA-DR10, HLA-DR1 and HLA-DR2 but absent in HLA-DR3, were obtained from the HLA-DR1 structure and used to complete the model. In the second approach, a hybrid model was generated using the atomic coordinates obtained from different segments of the HLA-DR 1, HLA-DR2 and HLA-DR4 crystal structures. The particular segments of the three HLA crystal structures used in the model were selected based on similarities in their secondary structural elements. Sequence-structure alignments were generated using the Smith-Waterman (Smith and Waterman (1981) J. Mol. Biol., 147:195-197), FASTA (Pearson (1991) Genomics, 11: 635-650), BLAST and PSI-BLAST (Altschul et al. (1997) Nucleic Acids Res., 25: 3389-3402) algorithms, and the backbone of the model was created automatically using the AS2TS system (see http://sb9.llnl.gov/adamz/LGA/AL2TS/as2ts.html website). The coordinates for the amino terminal four residues of the structure were taken from the 1seB crystal structure of HLA-DR1, residues 5-122 were obtained from the 1aqd structure of HLA-DR1, residues 123-170 were taken from the 1d5m HLA DR4 structure, and the remaining residues (aa171-193) were obtained from the 1bx2 structure of HLA-DR2. The construction of the terminal regions and loops, amino acid insertions and deletions, and template-model structure comparisons were performed using the LGA program developed at LLNL (see website http://predictioncenter.llnl.gov/local/lga/lga.html). The majority of the side chain atom's coordinates were incorporated from the four structural templates (listed above) due to their high level of homology. The side chains in selected regions of the protein model were built using the SCWRL program (Id.). Energy minimization was performed on both structures to eliminate inappropriate side chain contacts and the resulting structures were “optimized” using molecular dynamics.

Analyses of the resulting models revealed the two approaches yielded structures that were remarkably similar. Extended molecular dynamics runs appeared to provide little additional improvements. The results of the modeling revealed that the structure of the HLA-DR10 molecule is comprised of two domains linked by a hinge with one of the Lym-1 reactive residues, V85, positioned directly adjacent to the hinge.

The majority of the core of the relaxed structure of HLA-DR10, when compared with the HLA-DR3 crystal structure, was found to be essentially identical. The other three amino acids that were observed to play a role in Lym-1 binding, R70, R71 and A74 (A or E at this position appears important for Lym-1 binding), are all located on the exposed surface of a long alpha helix (FIGS. 9 and 10) located immediately adjacent to the hinge

B) Identification of Unique Sites on the Surface of HLA-DR10 within the Lym-1 Epitope that May be Targeted for Ligand Binding.

Solvent accessible surfaces of the HLA-DR10 protein and the crystal structure of HLA-DR3 were calculated using the atomic coordinates obtained for HLA-DR3 from the Protein Data Bank and the HLA-DR10 model. The site surrounding the three key amino acids in the Lym-1 epitope (within 6 Å) were examined and compared. These three amino acid changes in the HLA-DR10 sequence (Q70R, K71R, and R74A) change both the charge distribution and topography of the protein's exposed surface in this region.

A program developed to identify “pockets” on the surface of the protein (SPHGEN) was used to identify potential cavities that may be targeted for ligand binding. Details of the programs used are described in Example 2. Two adjacent pockets (cavities) in the modeled HLA-DR10 surface were selected as appropriate sites for ligand binding, based on their proximity to each other, the three Lym-1 reactive residues, and the uniqueness of the amino acids lining the pocket. An examination of the crystal structures of the other four HLA-DR molecules also showed that pockets exist at or in the near vicinity of these sites in each HLA-DR molecule, but as expected (based on the amino acid sequence differences in the peptide chains in this region of the HLA-DR structure) the pockets present on each HLA-DR differ in size, shape, and distance separating them. Since the amino acid “landscape” surrounding these two sites differs significantly in HLA-DR3 and HLA-DR10, docking runs performed at both sites would be expected to identify ligands that bind selectively to the pockets on HLA-DR10 but not to HLA-DR3. These sites, identified as Site 1 and Site 2 and shown filled with red and blue spheres flank both sides of the most important amino acid in the Lym-1 epitope, R70. A third unique site, which may be targeted as a backup if suitable ligands cannot be identified that bind to Site 1 or Site 2, has also been located and characterized.

C) Computationally Screening a “Virtual” Ligand Library to Identify Small Molecules that May Bind to Specific Pockets on HLA-DR10 Encompassing the Lym-1 Epitope.

The program DOCK (UCSF) was used to perform a “virtual” screen of the Available Chemical Directory database of small molecules to identify the top ranked 1,000 molecules predicted to bind in the two unique pockets identified as Site 1 and Site 2. Details of the computational docking procedures are described in Example 2.

The top ranked 2,500 molecules were then visually inspected to select down to thirty-five molecules for experiment binding assays (Table 5). This final selection process was based on chemical properties including hydrophobic interactions, hydrogen bonding, and molecular size, and well as practical criteria including commercial availability and cost, ease of synthetic linkage, the overall structural diversity of the set of molecules.

TABLE 5
Ligands predicted to bind to Site 1 on the beta subunit
of HLA-DR10 by computational docking

| No. | Compound |
|---|---|
| 1. | 7-Amino-4-chloromethylcoumarin, glycyl-1- |
| | proline amide, hydrochloride |
| 2. | 5-([4,6-Dichlorotriazin-2-yl]amino)-fluorescein hydrochloride |
| 3. | 2-[2-[3-Chloro-5-(trifluoromethyl)-2- |
| | pyridinyl]carbohydrazonoyl]benzene carboxylic acid |
| 4. | 4-[[2-(4-Cyano-3-phenyl-5- |
| | isoxazolyl)vinyl]amino]benzenecarboxylic acid |
| 5. | 4-[2-(2,4-Dichlorophenyl)hydrazino]-4-oxo-2-phenyl- |
| | 2-butenoic acid |
| 6. | 3-(2-[[3-Chloro-5-(trifluoromethyl)-2-pyridinyl]oxy]anilino)- |
| | 3-oxopropanoic acid |
| 7. | 3-[(4-Chlorobenzyl)thio]imidazo[1,5-a]pyridine-1-carboxylic acid |
| 8. | 2-[([1,1′-Biphenyl]-4-ylamino)carbonyl]benzoic acid |
| 9. | Bis[4-(3-aminophenoxy)phenyl]sulfone |
| 10. | 4,4′-Bis(4-aminophenoxy)biphenyl |
| 11. | 5(6)-Carboxytetramethylrhodamine n-succinimidyl ester |
| 12. | 1,4-Phenylenebis[[4-(4-aminophenoxy)phenyl]methanone] |
| 13. | H-Tyr(Br—Z)—OEt |
| 14. | ′7-Amino-4-chloromethylcoumarin, 1-alanyl-1-proline |
| | amide, hydrochloride |
| 15. | ′5-(N′-[2-aminoethyl]thioureidofluorescein) |
| 16. | Achatin I, Ammonium salt |
| 17. | Fmoc-Asp(OBzl)-OH |
| 18. | Fmoc-Bip-OH |
| 19. | Menai H535 |
| 20. | 2′-Methoxy-5′-methyl-3,4,5,6-tetrachlorophthalanilic acid |
| 21. | 4-Dimethylaminobenzene-4′-sulfonyl-1-valine |
| 22. | Bigchap |
| 23. | Arg-gly-asp-thr (SEQ ID NO: 16) |
| 24. | n-Allyl-2-[(1-benzyl-2-oxo-1,2-dihydro-3-pyridinyl)carbonyl]- |
| | 1-hydrazinecarbothioamide |
| 25. | n′-Methoxy-n-[7-(4-phenoxyphenyl)[1,2,4]triazolo[1,5- |
| | a]pyrimidin-2-yl]iminoformaraide |
| 26. | n-[[6-(4-Chlorophenoxy)-3-pyridyl]carbonyl]-n′- |
| | [3-(trifluoromethyl)phenyl]urea |
| 27. | n-[[6-(4-Chlorophenoxy)-3-pyridyl]carbonyl]-n′- |
| | (4-chlorophenyl)urea |
| 28. | n,n′-Diphenylbenzidine |
| 29. | Rcl s16, 963-3 |
| 30. | N,N′-bis-(4-amino-2-chloro-phenyl)-terephthalamide4- |
| | [[5-(trifluoromethyl)pyridin- |
| | 2-yl]oxy]phenyl N-phenylcarbamate |
| 31. | Methidiumpropyl ethylenediame tetraacetic acid |
| 32. | N-(4-[[3-Chloro-5-(trifluoromethyl)-2- |
| | pyridinyl]methyl]phenyl)-4-iodobenzenecarboxamide |
| 33. | 2-(4-Chlorophenyl)-2-[6-[(4-chlorophenyl)sulfanyl]- |
| | 3-pyridazinyl]acetamide |
| 34. | 6-Chloro-n4-(4-phenoxyphenyl)-2,4-pyrimidinediamine |
| 35. | 4-Amino-2-anilino-5-benzoyl-3-thiophenecarbonitrile |

This procedure was repeated for identification of thirty-five potential binders for Site 2. Table 6 lists those ligands predicted by molecular docking to bind to Site. 2.

TABLE 6
Ligands predicted to bind to Site 2 on the beta subunit of
HLA-DR10 by computational docking

| | |
|---|---|
| 1. | Leu-enkephalin |
| 2. | 4-[4-(4-Chlorobenzyl)piperazino]-3-nitrobenzenecarboxylic acid |
| 3. | Beta-casomorphin (1-2) |
| 4. | L-Aspartic acid, alpha-(4,5-dimethoxy-2-nitrobenzyl) |
| | ester, hydrochloride |
| 5. | Cefadroxil |
| 6. | 3,3′,5-Triiodo-dl-thyronine |
| 7 | H-Glu(anilide)-OH |
| 8. | H-Trp-phe-OH |
| 9. | Glycylglycyl-D,L-phenylalanine |
| 10. | Thymopoietin II (33-36) |
| 11. | Thiophosphoric acid S-(3-(3-amino-propylamino)-propyl) ester, |
| | di-hydrate |
| 12. | Dynorphin A (13-17), Porcine |
| 13. | 1-Alanyl-1-alanyl-1-tryptophan |
| 14. | Asp-arg-val-tyr (SEQ ID NO: 17) |
| 15 | A-VI-5 |
| 16. | Glu-thr-pro NH₂ |
| 17. | (D-ala2)-Beta-casomorphin (1-5) (bovine) |
| 18. | Tyr-D-ala-gly-phe-D-met acetate salt (SEQ ID NO: 18) |
| 19 | Arg-gly-asp-thr (SEQ ID NO: 19) |
| 20. | N-Alpha,n-omega-di-cbz-1-arginine |
| 21. | Asp-lys acetate salt |
| 22. | (+)-Allo-octopine |
| 23. | Sodium 7-[(2-amino-2-phenylacetyl)amino]-3-methyl-8-oxo-5- |
| | thia-1-azabic cyclo[4.2.0]oct-2-ene-2-carboxylate |
| 24. | Val-ile-his-asn_(SEQ ID NO: 20) |
| 25. | 2-Amino-8-(diphenylphosphinyl)-octanoic acid |
| 26. | Glu-His-Pro NH₂ |
| 27. | Bapaba |
| 28. | H-glu(lys)-OH |
| 29. | Bis-boc-1-arg |
| 30. | H-arg(mtr)-OH |
| 31. | 4-Aminomethyl-L-phenylalanine boc |
| 32. | H-met-met-OH |
| 33. | H-trp-ile-OH |
| 34. | N-alpha benzoyl-arginine-4-amino benzoic acid |
| 35. | (Thr46)-osteocalcin (45-49) (human) |

As a result of the computational docking, 30 compounds from both the Site 1 and Site 2 lists were experimentally screened by NMR. Eleven compounds were found to bind to HLA-DR10, giving a successful hit rate of 37%. These ligands are listed in Table 7.

TABLE 7
NMR screened ligands that were found to bind to HLA-DR10.
Ligands are separated by computationally predicted docking sites

| Site 1: | Site 2: |
|---|---|
| 5(6) carboxytetramethylrhodamine-n | N-alpha benzoyl-arginine- |
| succinimidyl ester | 4-amino benzoic acid |
| Methidiumpropyl EDTA | 5-leu-enkephalin (YAGFM) |
| Deoxycholic acid | N alpha N omega |
| FMOC-aspartic acid(O-benzyl)-OH | dicarbobenzoxyarginine |
| 4-dimethylaminoazobenzene-4′- | Angiotensin II (DRVY) |
| sulfonyl-L-valine | Bis-BOC-L-arginine |
| 4-[[5-(trifluoromethyl)pyridin-2- | |
| yl]oxy]phenyl | |
| N-phenylcarbamate | |

From Table 7, 5 synthetic high affinity ligands (SHALs) have been synthesized, containing different sets of the Site 1 and Site 2 ligands. Three of these molecules all containing the ligand pairs deoxycholate and 5-leu-enkephalin, have been shown to bind to isolated HLA-DR10. None of these three SHALs bind to albumin or streptavidin. The first of the three to be tested more extensively, JP459B has been determined to bind to HLA-DR10 with a Kd=23 nM using Surface Plasmon Resonance. Using Raji membrane extracts, this SHAL competed with Lym-1 for binding to HLA-DR10.

In subsequent screenings additional ligands have been shown to bind to HLA-DR10. These are shown in Table 8.

TABLE 8
Additional ligands that bind to HLA-DR10.

| Ligand | |
|---|---|
| ID | Species |
| 11 | 3,3′,5-Triiodo-dl-thyronine (Predicted Site 2) (TI) |
| 7 | 2-(4-Chlorophenyl)-2-[6-[(4-chlorophenyl) suflfanyl]- |
| | 3-pyridazinyl] acetamide (12F) |
| 9 | 4-Amino-2-anilino-5-benzoyl-3-thiophenecarbonitrile (5K) |
| 8 | 6-Chloro-n4-(4-phenoxyphenyl)-2,4-pyrimidinediamine (7L) |
| 6 | N-(4-[[3-Chloro-5(trifluoromethyl)-2-pyridinyl]methyl]phenyl)- |
| | 4-iodobenzenecarboxamide (6J) |

In addition 1,4-phenylenebis[[4-(4-aminophenoxy)phenyl]methanone] precipitated onto the target protein.

### Example 2

### Computational Methods for Use in the Creation of SHALS

### A) Overview of the Roles and Methods of Molecular Simulations

As described herein, molecular modeling may be used to initially identify ligands for use in the construction of SHALs and/or for the optimization of SHALs. At present there is no single molecular modeling methodology that may be used to model target molecules, screen for binding ligands, simulate binding of a polydentate SHAL and predict optimal SHAL structure. A number of well established modeling methods, however may be used to facilitate these tasks as described herein.

Starting at the highest level, the prediction of the tertiary structures target molecules (e.g., protein cancer markers) are typically predicted using highly empirical methods based on primary sequence homology to proteins with experimentally known structure. The accuracy of these so-called homology-based protein structure prediction methods depend on the availability of homologous protein structures and the expertise of the individual modeler. To identify small molecules (ligands) that specifically bind into protein pockets, computational “docking” may be employed as described herein. Docking uses a relatively simple empirical force field to describe the ligand-protein interaction and may therefore be used to rapidly screen 100,000's of possible ligands. To determine preferred macromolecular conformations and interactions, classical molecular dynamics may used, which models the molecules using empirical ball-and-spring force fields. Finally, for the precise prediction of small molecule structures and interactions, one may computationally solve the quantum mechanical equations describing the electrons and nuclei within the molecules. This so-called first principles approach may either be used to determine the structures and energies of static “snapshots” of the molecules or to simulate the atomic motions of the molecular systems. The former approach, is referred to as ab initio quantum chemistry while the latter approach is called first principles molecular dynamics (in contrast to classical molecular dynamics) and constitutes a nearly exact simulation of nature.

### B) Homology-Based Protein Structure Predictions.

The basic concept of homology-based protein structure prediction relies on the observation that structural features of proteins are conserved during evolution to a much higher degree than their sequences, and therefore proteins related even by distant sequence similarity may be expected to have similar 3D structures (Chothia and Lesk (1986) EMBO J., 5: 823-826). Thus, once a three-dimensional structure is determined for at least one representative of a protein family, models for other family members may be derived using the known structure as a template. Homology-based protein modeling consists of four major steps: finding known structures related to the protein sequence to be modeled, aligning the sequence with these structures, building a three-dimensional model, and assessing the model (Marti-Renom et al. (2000) Annu Rev Biophys Biomol Struct., 29: 291-325).

Homology-based protein structure prediction (also referred to as comparative modeling) produces an all-atom model of a sequence based on its alignment with one or more related protein structures. Building of the three-dimensional model itself includes either sequential or simultaneous modeling of the core of the protein, loops and side chains.

The accuracy of a model, built using comparative modeling technique, usually is related to the percentage of sequence identity with the structure on which it is based. High-accuracy comparative models are based on more than 50% sequence identity to their templates. They tend to have about 1 Å root-mean-square (RMS) error for the main chain atoms. Such accuracy is comparable with medium resolution nuclear magnetic resonance (NMR) structure or low resolution X-ray structure. The errors in such cases are usually limited to mistakes in side chain rotamer assignment, small shifts or distortions in the core main chain regions, and occasionally larger errors in loops.

One general modeling approach will be similar to that successfully used earlier to model both high and low homology target proteins (Venclovas et al. (1999) Proteins—Structure Function and Genetics, 73-80). Since the modeling objects (e.g., HLA-DR10) may have high sequence homology (>50% sequence identity) we may rely on pairwise sequence comparison of modeling target (query) with the proteins of known structures (from the Protein Data Bank (PDB)) to identify the closest structural templates. To do this, a sensitive Smith-Waterman pairwise sequence comparison algorithm (Smith and Waterman (1981) J. Mol. Biol, 147: 195-197) implemented in the SSEARCH program (Pearson (1991) Genomics 11: 635-650) may be used. At the high level of sequence homology structure alignment for the conserved structural regions may be used directly in model-building.

When a number of structural templates of comparable similarity are available one may use MODELLER, a comparative modeling program capable of automatically combining a number of template structures to better represent the structure of the query. Where critical regions are present in the target molecule, special care may be taken in assigning conformations to these regions. The candidate conformations for these regions may be produced by searching a database of homologous structures for the fragments of identical length that also satisfy the steric constraints for these regions. Both sequence similarity and structural context near the region may be taken into account in selecting the actual conformation. Side chains within the model may be positioned using a backbone-dependent rotamer library (Bower et al. (1997) J. Mol. Biol. 267: 1268-1282).

Assessment of the obtained models may be done using several techniques. One of these, Prosall (Sippl (1993) Proteins, 17: 355-362; Aloy et al. (2000) J Comput Aided Mol. Des. 14: 83-92), which is used to detect errors in protein structures, creates an energy profile along the sequence of the protein. The regions that are assigned high energy values by Prosall often serve as good indicators of errors in representing the structure of these particular regions. For the detailed checks of modeled structures, the structure verification module of the WHATIF program (Vriend (1990) J. Mol. Graph. 8: 52-56) may be used along with visual inspection. If these assessments of model quality identify any problems in the modeled structure, appropriate steps (such as loop assignment or side chain positioning) will be repeated in an iterative manner until an acceptable quality three-dimensional model is obtained.

Using these methods a computer model of the molecular structure of the HLA-DR 10 beta subunit containing the region shown to be critical for Lym-1 antibody binding to malignant B cells was developed and the structure was compared with the structure with other HLA-DR molecules (see Example 1, supra.). This model was used to develop HLA-DR10 specific SHALs as described herein.

### C) Computational Docking

Computational methods such as docking have been used to speed up the process of drug discovery and inhibitor design by screening large numbers of molecules and predicting whether or not they bind into the active sites of target proteins (Desjarlais et al. (1990) Proc. Natl. Acad. Sci. U.S.A., 87: 6644-6648; Mao et al. (1998) Bioorganic and Medicinal Chem. Letts., 8: 2213-2218; Olson and Goodsell (1998) Environmental Res., 8: 273-285; Rutenber et al. (1993) J. Biol. Chem., 268: 15343-15346). These efforts have met with moderate success in the design of new drugs effective against HIV proteins critical for infection and transmission of the disease

In certain embodiments, this approach is generally useful as a first step in the identification of ligands (binding moieties) that usually bind to the target molecule(s) in the micromolar range. Detailed protocols for docking methods using SPHGEN and DOCK have been described in the literature. For example, these methods have been used to identify ligands that bind to specific sites on the targeting domain of tetanus neurotoxin (Cosman et al. (2002) Chem Res Toxicol 15: 1218-1228; Lightstone et al. (2000) Chem. Res. Toxicol., 13: 356-362).

The program DOCK 4.0 was used to computationally screen the Available Chemical Directory (^{˜}300,000) of small molecules to identify the top ranked 2,500 molecules predicted to bind to the identified Site 1 and Site 2.

Computational docking may be thought of as a three-step process: 1) site identification of the protein surface; 2) docking of ligands into the identified binding site; and 3) scoring and the ranking of the ligands (Halperin et al., (2002) Proteins: Structure, Function, and Genetics, 47: 409-443). For site identification, the solvent accessible surface of the target protein is generally calculated. Using the program SPHGEN, a utility in DOCK (Moustakas and Kuntz (2002). DOCK5.0 (San Francisco, UCSF)), concave pockets on the protein surface were identified by filling the pockets with different sized radii spheres. Essentially, this calculates the volume of the pocket. The surface of the protein may have anywhere from thirty to hundreds of pockets based on the size and shape of the protein. Once these pockets were identified, visual inspection of the pockets identified the binding site based on the size of the pocket and the available experimental evidence, such as known amino acids involved in binding or catalysis. The chosen binding pocket was then used in the subsequent docking procedure.

Docking studies identify small molecules that might bind specifically to the chosen binding site on the protein. The DOCK 5.0 program screens a database of compounds on the computer and predicts which molecules will likely bind tightly to the binding site. We used the Available Chemicals Directory (ACD) from MDL as the database of compounds to screen.

The database was prepared by prefiltering to remove soaps and dyes. After the partial charges for the compounds were calculated by using Gasteiger-Marsili charges (Gasteiger and Marsili (1978) Tetrahedron Letts., 34: 3181-3184; Gasteiger and Marsili (1980) Tetrahedron 36: 3219-3288; Gasteiger and Marsili (1981) Organic Magnetic Resonance 15:353-360) in Sybyl, the database was divided by total compound charge, and compounds with formal charge >±3 are filtered out. Also, compounds <10 and >80 heavy atoms (not hydrogens) were removed to focus on compounds within the size range for lead (preliminary) drug compounds. This prefiltering made the database more efficient and eliminated unnecessary calculations on compounds known to either never bind or bind indiscriminately. To simulate a flexible docking technique, 20 unique conformations were generated for each compound in the database. Each of these conformations was then rigidly docked into the binding site. Different orientations within the binding site were examined for each of the conformations of each of the ligands. All compounds were scored by energy minimization where the intermolecular van der Waals and electrostatic terms are derived from AMBER (Weiner (1984) J. Am. Chem. Soc., 106: 765-784). Though the molecules are ranked based on the scores, the scoring function does not predict the binding affinities.

The top ranked 2,500 molecules were then visually inspected to select down to thirty-five molecules for experiment binding assays as described in Example 1. Ligands were selected and bidentate SHALs were constructed and tested as described in Example 1.

The binding of lead compounds to the target may be improved by several orders of magnitude by using multiple (2-3) compounds linked together. For the inhibitor to be effective, it needs to recognize specifically the target protein and bind with high affinity.

### D) Quantum Chemical Calculations

A wide variety of chemical simulation methods have been developed, ranging from empirical ball-and-spring type molecular mechanics models to ab initio (first principles) quantum chemical methods that calculate approximate solutions to the exact quantum mechanical equations describing the electrons and nuclei. Typically, the choice of methods involves trade-offs between accuracy, size of the chemical system, and computational cost. These modeling methods may be broadly divided into molecular dynamics methods that simulate the time evolution of chemical processes and static methods that predict time-independent molecular properties such as the lowest energy configuration of a molecule or the energy of a chemical reaction. One may use all three molecular modeling methods described below: ab initio quantum chemistry, classical molecular dynamics and first principles molecular dynamics in the design and optimization of SHALs as described herein.

### 1. Ab Initio Quantum Chemistry (QM)

Ab initio quantum chemistry involves computing approximate solutions to the exact non-relativistic Schroedinger equation describing a molecular system (Jensen (1999) Introduction to Computational Chemistry, New York, John Wiley and Sons). In principle these methods may predict the properties of any chemical system to arbitrary accuracy, but in practice the computational cost limits the accuracy of these methods and the size of the molecular systems to which they may be applied. Nevertheless, ab initio quantum chemical calculations are routinely applied to calculate accurate structures and reaction energies for molecular systems including up to hundreds of atoms.

There is a hierarchy of different ab initio quantum chemical methods involving increasingly accurate mathematical descriptions of the electronic wave function—the mathematical description of the distribution of electrons around the nuclei of a molecule (Id.). Application of quantum chemistry typically requires the choice of both the description of the electron-electron interactions (level of theory) and the spatial flexibility of the electrons (basis set). A fairly new class of methods called Density Functional Theory (DFT) has been developed that includes empirical parameterizations of the electron-electron interactions, and often provides accuracy comparable to the earlier high-level quantum chemical methods (such as Coupled Cluster methods), but with a much lower computational cost. The DFT methods are usually denoted by the empirical electron-electron “functional” employed. Two widely used DFT functionals are the Becke 3-parameter hybrid exchange functional (Becke (1993) J. Chem. Phys. 98: 5648-5652) and the Lee-Yang-Parr gradient corrected electron correlation functional (Lee et al. (1988) Chemical Physics 123: 1-25). These have been widely demonstrated to yield accurate chemical structures and reaction energies for most molecules when used with sufficient basis sets (Jensen (1999) Introduction to Computational Chemistry, New York, John Wiley and Sons).

The quantum chemical simulations described herein are used to study chemical processes that occur in the immediate extracellular environment. The quantum chemical methods described above typically describe only an isolated (usually described as “gas-phase”) molecule and therefore do not include the chemical environment, such as solvent molecules and counterions, which frequently is critical to the structure and energetics of biological molecules. Explicitly including the surrounding water molecules and counter ions is usually not computationally practical; however, several methods have been developed within the quantum chemistry approach for effectively including the effects of solvent interactions. Typically these methods model the solvent as a continuous medium that polarizes in response to the quantum chemically derived charges. Although there are many situations where explicit inclusion of the solvent is necessary, these so-called polarizable continuum models have proven reasonably accurate in predicting solvent-phase chemical properties including total solvation energies and acid constants (Schüürmann et al. (1998) J. Physical Chemistry A 102: 6706-6712; Tran and Colvin (2000) J. Molecular Structure, Theochem 532: 127-137).

The Langevin dipole method of Warshel is related to these polarizable continuum models, but includes a more realistic representation of the polar solvent. The Langevin dipole method models the solvent as a large set of polarizable dipoles on a fixed three-dimensional grid (Luzhkov and Warshel (1992) J. Computational Chemistry 13: 199-213). This approach has recently been parameterized for use with ab initio derived solute charges and shown to yield solvation energies for neutral and ionic molecules comparable or better than PCM methods described above (Florian and Warshel (1997a) J Am Chem. Soc., 119: 5473-5474).

### 2. First Principles Molecular Dynamics (FPMD)

By combining the forces determined directly from a QM method to drive the classical motion of all the atoms in a simulation, one may achieve the accuracy of quantum mechanics (QM) with the advantages of classical molecular dynamics. This approach became computationally feasible with the development of a new technique based on density functional theory (DFT) (Kohn Sham (1965) Physical Review 140: A1133) that treats electronic degrees of freedom at the same time as the nuclear equations of motion (Car and Parrinello (1985) Physical Review Letters 55: 2471-2474; Galli and Parrinello (1991) Pp. 283-304 In: Computer Simulation in Materials Science, The Netherlands, Kluwer Academic Publishers). Since the method employs QM theory to describe the entire system, it is often referred to as first principles molecular dynamics (FPMD). In the typical implementation of FPMD, only the chemically active valence electrons are explicitly described with an expansion in a plane-wave basis, while the chemically inert core electrons are represented by pseudopotentials (Galli and Pasquarello (1993) Pp. 261-313 In: Computer Simulation in Chemical Physics, D. J. Tildesley, ed. Dordrecht, Kluwer; Yin and Cohen (1982) Physical Review B (Condensed Matter) 25: 7403-7412). Because the pseudopotentials are transferable by design, this method does not require reparameterization when new systems are studied. In addition, the use of a plane wave basis set naturally lends itself to the application of periodic boundary conditions, so the method is well suited for modeling systems in the condensed phase. This method, combined with several other computational improvements (Gygi (1993) Physical Review B 48: 11692-11700; Hutter et al. (1994) Computational Materials Science 2: 244-248; Payne et al. (1992) Rev. Modern Physics 64: 1045-1097), has been instrumental in solving the problem of integrating QM and MD.

The first applications of FPMD simulations were limited to small systems such as silicon (Car and Parrinello (1985) Physical Review Letters 55: 2471-2474; Stich et al. (1989) Physical Review Letters 63: 2240-2243). As these methods have been continuously improved upon, and advanced computational resources have become available (such as the DOE teraflop scale supercomputers) it is now possible to investigate small biochemical systems containing several hundreds of atoms for picosecond timescales (Carloni and Alber (1998) Perspectives in Drug Discovery and Design 9/11: 169-179; Pantano et al. (2000) J. Molecular Structure (Theochem) 530: 177-181; Rovia and Parrinello (2000) International Journal of Quantum Chemistry 80: 1172-1180). For example, we have recently simulated the conformational dynamics of a small chemical model of the DNA backbone in solution. As the number of systems that have been investigated with this new approach increases, it is becoming clear that the increased computational expense is repaid in the form of extremely accurate structural and dynamical properties. In particular, such methods potentially allow for very accurate dynamical simulations of chemical phenomena including chelator-metal ion interactions and enzyme-catalyzed reactions.

### E) Classical Molecular Dynamics (MD)

Classical molecular dynamics may be used to in identifying the exact orientation of the ligands in the in the binding sites of the target molecule(s) (e.g., HLA-DR 10 binding sites (Site 1 and Site 2)). This information may be used in designing the multivalent ligands to carry radioisotopes selectively to the target molecule and/or to cells displaying the target molecule.

In particular, this structural data helps identify which functional groups on the ligand(s) may be used to synthetically attach the linker. For all ligands that are experimentally verified to bind to the target, classical molecular dynamics may be performed on the ligand in the target binding sites to determine conformation and orientation of the ligand and the specific interactions mediating the binding specificity (e.g., hydrogen bonds, electrostatic and Van der Waals interactions). In certain embodiments, the molecular dynamics simulations will include the target and the ligands solvated in a periodic water box. For each ligand, 500 ps to several nanosecond simulations may be performed using multiple starting orientations.

An important component of the polydentate SHALs of this invention is the molecular linker between the individual ligands (binding moieties) comprising the SHAL. Typically this linker adopts an aqueous-phase conformation (or set of conformations) that holds the two (or more) ligands at appropriate distances to efficiently bind into their binding site on the target surface. To assist in designing this linker classical molecular dynamics may be performed on the linker molecular alone and the linker bound to specific ligand compounds.

The program CHARMM may be used to perform classical molecular dynamics simulations on various linkers. For example, simulations performed with PEG linkers may utilize a different number of PEG units (4, 6, and 8) in the molecules. The starting structures for all four simulations may have the molecules in a fully extended conformation. These extended molecules may be solvated in water boxes and sodium ions were added to each water box to neutralize the systems. These solvated systems were heated to e.g., to 300 K and allowed to equilibrate for 200 picoseconds. Typically simulations may be run at constant temperature (NVT ensemble) and electrostatic interactions treated by particle mesh Ewald (PME) summation.

Initially the two compounds to be linked may be visually examined together bound to the protein as determined by docking and molecular dynamics using computer graphics. For example, a polyethylene glycol (PEG) linker will be built using molecular drawing software (AMPAC) between the two compounds. This structure may then be simulated using classical molecular dynamics in a periodic water box, e.g., as described above for several nanoseconds. One may analyze the resulting data on the dynamical motions to measure the average ligand-ligand distance and relative ligand orientation. This data may be compared with the distance between the ligands and relative orientation on the target 10 surface to determine the optimal length for the linker.

Molecular dynamics simulations may also be used to investigate a number of other properties on the overall SHALs to optimize their therapeutic effectiveness. In particular molecular dynamics simulations may be used to investigate a number of modifications including the chemical structure of the linker itself, and the two ligands at each end. The goal of these simulations will be to identify likely effects of such modifications on target binding efficiency and specificity prior to expensive synthetic modifications.

Molecular dynamics methods use an empirically derived classical force field to simulate the motion of each atom in a chemical system. This methodology is highly developed for the simulation of nucleic acids, (Beveridge and McConnell (2000) Curr. Opinion in Structural Biology 10: 182-196) and proteins (Brooks et al. (1983) J. Computational Chemistry 4: 187-217; Cheatham and Brooks (1998) Theoretical Chemistry Accounts 99: 279-288; Doniach and Eastman (1999) Curr. Opinion in Structural Biology 9: 157-163). Typical published molecular dynamics simulations involve 10-100,000 atoms (including both the biomolecules being simulated and a surrounding shell of water and counter ions) which are simulated for multi-nanoseconds of time, with the largest published simulation being a 1 microsecond simulation of a small protein (Duan and Kollman (1998) Science 282: 740-744). The multinanosecond time scale is thought sufficient to capture structural relaxation and solvent reorganization, and is long enough in some cases to simulate transitions between different macromolecular conformations (Cheatham and Kollman (1996) J. Mol. Biol., 259: 434-444; Yang and Pettitt (1996) J. Physical Chemistry A 100: 2564-2566).

A molecular dynamics simulation of polyethylene glycol (PEG) has recently been published that is relevant to the design of PEG linkers for SHALs. Heymann and Grubmuller used classical molecular dynamics to describe the conformational and elastic properties of individual PEG chains (Heymann and Grubmuller (1999) Chemical Physics Letters 307: 425-432; Young and Lovell (1992) Introduction to Polymeres, New York, Chapman and Hall). They simulated a PEG 18-mer (^{˜}1 kDalton molecular weight) in the aqueous phase (solvated by 1539 water molecules) and in the gas-phase (to approximate solvation in a non-polar solvent such as hexadecane). They found that in the gas-phase the PEG rapidly collapsed to a compact structure with no local structure, as measured by the degree to which the PEG had a helical local structure. In water, the PEG behaves very differently. It does show a reduction in the radius of gyration compared to the fully extended structure, but retains a marked degree of helicity and therefore some degree of stiffness. These simulations indicate that the local stiffening of the PEG structure is caused by water molecules that form hydrogen bond bridges between successive oxygens in the PEG chain. They further simulated the stretching of the PEG chain with a range of forces from 0 to 500 picoNewtons (this mimics experimental studies with Atomic Force Microscopes). They find good agreement in their predicted force versus extension curves with values recently measured in a single PEG molecule (Oesterhelt et al. (1999) New Journal of Physics 1: 6.1-6.11).

These results demonstrate that classical molecular dynamics simulations of PEG may accurately reproduce complex properties such as the force/extension curves and strongly supports the accuracy of the proposed PEGylated scaffold simulations. Although the PEGylated scaffold currently in use (13.6 kDalton molecular weight) is considerably larger than the PEG 18-mer simulated by Heymann and Grubmiller, it is well within reach of routine molecular dynamics simulations.

Molecular dynamics simulations may readily be performed with the CHARMM software package (Brooks et al. (1983) J. Computational Chemistry 4: 187-217) using the version 22 parameter set (MacKerell et al. (1998) J. Physical Chemistry B 102: 3586-3616). Analysis may be performed using the analysis tools distributed with CHARMM and VMD, a graphical molecular dynamics analysis tool (Humphrey et al. (1996) J. Molecular Graphics 14: 33-38).

The steps in a typical setup and simulation runs are as follows:

### A. Preliminary Setup.

1. Calculation of partial charges for atom types not included in CHARMM force field. Model compounds containing the unparameterized atom types will be optimized at the Hartree-Fock level of theory using a 6-31G(d) basis set. Upon convergence, partial charges of each atom will be computed using Merz-Kollman charge fitting scheme (Besler et al. (1990) J. Computational Chemistry 11: 431-439). These charges may replace the default atomic charges.

### 2. Molecules to be Simulated:

a. Construction of Molecular Structures

The molecules to be simulated may be built using QUANTA and the atomic charges will be obtained as in step one above. The net charge of the whole compound may then be computed.

b. Solvation of the Molecular Structures:

The molecules and molecular complexes constructed in step 2a may be neutralized using Na+ ions that are positioned using the SOLVATE program. The whole system may then be solvated in a box of water molecules and this simulation box may be subsequently adjusted to yield the appropriate density.

### B. Running the Simulation:

### 1. Equilibration of the Molecule/Water/Counterion System:

a. Minimization: To remove residual strain remaining in the molecular structures from the construction phase, the solvated molecules from step 2b above may be minimized for 10,000 steps, of which the first 1,000 iterations are performed using steepest descent and the rest using adopted basis Newton-Raphson methods.
b. Equilibration: After minimization, The temperature may be ramped up from OK to 300K over 10 ps and held fixed at 300K thereafter. The system may be equilibrated for 200 ps at constant temperature. The long range forces are handled by particle mesh Ewald method Essmann et al. (1995) J. Chemical Physics 103: 8577-8593. The water molecules are TIP3P (Jorgensen et al. (1983) J. Chemical Physics 79: 926-935). An integration time step of 2 femtoseconds may be used, and the SHAKE algorithm may be employed to restraint all the motions of the hydrogen atoms (Reichert and Welch (2001) Coordination Chemistry Reviews 212: 111-131).
c. Production runs: For the production simulations, constant temperature molecular dynamics (using the NVT ensemble) may be used. The particle mesh Ewald method (Essmann et al. (1995) J. Chemical Physics 103: 8577-8593) may be used for the long range forces. During the dynamics runs, the complete set of atomic coordinates may be saved every 0.1 ps for subsequent analysis. For the preliminary simulations, the molecular dynamics simulations ran on a Compac Alpha computers at a speed corresponding to approximately 625 cpu hours (^{˜}4 weeks) per nanosecond, therefore, multinanosecond simulations of these systems will be routinely feasible on a large network of workstations.

### Example 3 Synthesis and Testing of a Bi-Denatate Shal

The bivalent SHAL (LeacPLD)2LPDo was synthesized, purified by HPLC and characterized by mass spectrometry. This SHAL has two JP459B bidentate ligands interconnected via a linker and a DotA attached on the third arm.

SHALs were designed around an orthogonally protected lysine residue to facilitate synthesis on solid phase resin. A commercially available Fmoc-protected amino acid-like mini-peg (2 CH2O's) was used as a linker to incrementally increase the distance between the enkephalin and the deoxycholate moieties. Fmoc-biotinyl-lysine was used to introduce biotin into the SHALs for biacore experiments. All SHALs follow the same configuration: CO2H:Biotin-lysine:lysine: (a lysine NH2: 0, 1, 2 mini-peg linker, deoxycholate)(g lysine NH2: LFGGY-NHAc). The bis-bidentate SHAL follows the convention: CO2H:Biotin-lysine:lysine:[(a lysine NH2: 0, 1, 2 mini-peg linker, deoxycholate)(g lysine NH2: LFGGY-NHAc)]2- and therefore is unsymmetrical about the second lysine residue.

All chemicals used were purchased from Aldrich or Nova Biochem. SHALs were synthesized using standard Fmoc solid phase synthesis on chlorotritylchloride resin. Ligands were cleaved from the resin and the protecting groups removed using the appropriate reagents. Trifluoroacetic acid esters formed on the primary alcohols of deoxycholate during cleavage from the resin were removed by stirring in ammonium bicarbonate. SHALs were purified using reverse phase high performance liquid chromatography (HPLC). Analytical HPLC was carried out at 1 mL/min on an Agilent 1100 machine (Waters Symmetry C18, 5 mm, 4.2×150 mm column) and preparative HPLC was carried out at 10 mL/min on a Waters preparative machine (Waters Symmetryprep C18, 7 mm, 19×300 mm column). SHALs were characterized using nuclear magnetic resonance (NMR) spectroscopy and electrospray mass spectrometry. 1H and 13C NMR spectra were recorded on a Bruker DRX 500 MHz spectrometer. Mass spectra were acquired on a Micromass Quattro Micro API mass spectrometer operating in positive ion mode.

Mass spectrometry of the (LeacPLD)2LPDo showed that it does not contain any free DotA. Free DotA would not be expected to be present based on the process used to synthesize the SHAL. (LeacPLD)2LPDo was synthesized by attaching each linker component or ligand onto a growing chain covalently attached to the surface of a resin. After each chemical reaction the resin was extensively washed to remove the unreacted products. DotA was attached to the linker at the beginning of the synthesis. After the excess DotA was washed away, multiple additional chemical reactions that were carried out on the resin to add the various linkers and ligands, and after each reaction the unreacted products were again washed away. By the time the synthesis of the SHAL was completed, the amount of free DotA present in the sample was undetectably examination of the HPLC and mass spectrum. The DotA link is extremely stable, so it does not come off the SHAL once it's been attached.

During radiochemistry, components that may be present in the early runoff peak that may have associated radioisotopic label include the buffer salt (ammonium bicarbonate), possibly a trace amount of trifluoroacetate removed from the SHAL hydroxyls during the final synthesis step, and excess EDTA and free and EDTA-complexed isotope that didn't bind to the DotA. Dialysis may simply not be the preferred method for efficiently eliminating all this material. Reverse dialysis is one preferred method for purification. Alternatively, chelate (EDTA) scrubbing after radiochemistry may be performed using an EDTA bead column to remove radiometal that has not been DotA chelated or is loosely attached in a non-specific manner.

To get the metal into the DotA efficiently, adjusting the reaction mix to an adequate alkaline pH is also important. Since the SHAL as a molecule is quite different from an antibody-DotA molecule, the method used to raise the pH on the SHAL-DotA complex preferably also raises the pH sufficiently on the SHAL. One may easily get other metals in DotA if they are present at any stage. These may be detected by checking the mass spectrum of the compound. Observations into the spectrum from purified SHALs and has shown little or no other metal there.

Biotinylated deoxycholate-iodothyronine SHALs are synthesized in an analogous manner.

ELISA assays showed that SHAL, LeacPLBD (the univalent bidentate SHAL), bind to and discriminate between cells containing HLA-DR10 and those that do not contain HLA-DR10.

Several pharmacokinetic, biodistribution and imaging studies were performed with consistent results. In certain embodiments, 111In-DotA [SHAL 070804(LeacPLD)2LPDo] biodistribution was determined in Raji tumored mice.

### Background

Several strategies have been used to selectively deliver toxic chemicals or radiation to cancer cells (DeNardo (2005) Semin Oncol., 32:S27-35; Torchilin (2007) AAPS J., 9:E128-147), for gene therapy (Jeong et al. (2005) J Control Release 107:562-570; Xia et al. (2007) J Gene Med 10:306-315) or as tools for transfecting cells (Shigeta K (2007) J Control Release 118:262-270) and silencing genes (Liu (2007) Brief Funct Genomic Proteomic 6:112-119). Some of the earliest approaches used to enhance the cellular uptake of therapeutics and other molecules (fluorescent dyes, enzymes, antibodies and other proteins) involved introducing the molecules into liposomes or micelles (Constantinides et al. (2008) Adv Drug Deliv Rev 60:757-767; Samad et al. (2007) Curr Drug Deliv 4:297-305). Such constructs have been shown to fuse with the cell's membrane, introducing the contents inside the cell or transferring the lipid-bound components into the cell's membrane. Another highly successful approach has been to develop antibodies that target cell-specific membrane proteins and to use these antibodies to deliver radionuclides or other cytotoxic molecules to the surface of a specific population of cells (Brumlik et al. (2008) Expert Opin Drug Deliv 5:87-103; DeNardo et al. (1998) Cancer Biother Radiopharm 13:239-254; Tolmachev et al. (2007) Cancer Res. 67:2773-2782). More recently, intracellular delivery has been accomplished by attaching the molecules to be transported to naturally occurring transmembrane “shuttles”, peptides or proteins that readily pass through cellular membranes. One of the more successful shuttles is a nuclear localization signal peptide derived from the SV40 T antigen (Yoneda (1997) J Biochem 121:811-817). This sequence, other peptide sequences derived from the transduction domain of the HIV-1 protein Tat (Schwarze et al. (1999) Science 285:1569-1572; Torchilin et al. (2003) Proc. Natl. Acad. Sci., USA, 100:1972-1977), penetratin (Tseng et al. (2002) Mol Pharmacol 62:864-872), and intact proteins such as the herpes virus protein VP22 (Phelan et al. (1998) Nat Biotechnol 16:440-443) and anti-DNA antibodies (Avrameas et al. (1998) Proc. Natl. Acad. Sci., USA, 95:5601-5606) are currently being used to facilitate the transport of liposomes, viruses, enzymes, antibodies and a variety of other proteins into cells. Considerable success has also been achieved using synthetic cationic peptide transporters such as oligoarginine (Futaki (2005) Adv Drug Deliv Rev 57:547-558; Han et al. (2001) Mol Cells 12:267-271; Kim et al. (2007) Int J Pharm 335:70-78; Tung and Weissleder (2003) Adv Drug Deliv Rev 55:281-294), lactosylated poly-L-lysine (Midoux et al. (1993) Nucleic Acids Res 21:871-878) and short peptide sequences selected from phage display libraries (Kamada et al. (2007) Biol Pharm Bull 2007, 30:218-223) that exhibit sequence similarities to know peptide shuttles.

Recently, several small molecule antibody mimics that show promise as targeting agents for cancer imaging or therapy have been synthesized [24-28]. In addition to exhibiting selectivities and affinities (nM to pM) similar to antibodies, these molecules have the potential to minimize some of the difficulties associated with the use of protein-based drug delivery systems. They retain the more desirable pharmacokinetic properties of small molecules, are less likely to be immunogenic, may prove stable enough for oral delivery, and the costs associated with producing the drug may be reduced significantly. The SHAL family of antibody mimics may also be easily modified to carry radioactive metals, a variety of tags that enable their use as imaging agents, and other small molecules (e.g. toxins or inhibitors). Another potentially useful modification includes alterations that facilitate uptake and internalization of the SHAL by the targeted cell, which would be expected to both increase tumor residence time and deliver the SHAL into an environment (the cytoplasm or nucleus) where it may cause additional damage.

Working with a SHAL developed previously for targeting HLA-DR10, an abundant cell surface receptor over-expressed on B-cell malignancies, a peptide analog to the SHAL may be synthetized by conjugating it to hexa-arginine, a peptide that has been demonstrated previously to facilitate the transport of proteins and nucleic acids into cells. Binding studies conducted with the SHAL and its hexa-arginine analog in vitro using HLA-DR10 expressing Raji cells show that the hexa-arginine sequence changed the SHALs properties significantly, enhancing both SHAL internalization and radionuclide residualization.

### Methods

### SHAL Design.

The process used to create a homology model for HLA-DR10, identify unique binding cavities within the Lym-1 epitope, select ligands that bind in these cavities, and create the (DvLPBaPPP)2LLDo SHAL has been reported previously (Balhorn et al. (2007) Clin Cancer Res 13:5621s-5628s). A process for producing a hexa-arginine peptide analog of this parent SHAL, (DvLPBaPPP)2LArg6AcLLDo, was developed by modifying the synthesis to include the incorporation of an additional lysine residue into the middle of the linker connecting the two SHAL monomers and attaching an arginine hexapeptide to the free amine on this lysine. SHAL Synthesis.

The two dimeric SHALs (DvLPBaPPP)2LLA and (DvLPBaPPP)2LArg6AcLLA were synthesized on chlorotrityl chloride resin using orthogonally protected lysine (L) residues and miniPEGs (P) to link the two small ligands Dv and Ba as previously described for (DvLPBaPPP)2LLA (Balhorn et al. (2007) Clin Cancer Res 13:5621s-5628s; Hok et al. (2007) Bioconjug Chem 18:912-921). To produce the amine derivative of the hexa-arginine SHAL (DvLPBaPPP)2LArg6AcLLA, a second Dde-D-Lys(Fmoc)-OH lysine residue was inserted into the linker during SHAL synthesis by performing two sequential Dde-D-Lys(Fmoc)-OH coupling steps. At the alpha position of the third lysine, six consecutive arginine residues were inserted by reacting the resin with Fmoc-D-Arg(Pbf)-OH six times. The sixth Arg residue was protected with an acetate (Ac) by reacting with acetic anhydride in N,N diisopropyl-ethylamine (DIEA)/dimethylformamide (DMF). The guanidinium groups on all six arginine residues remain protected with trifluoroacetic acid (TFA)-sensitive 2,2,4,6,7-Pentamethyldihydrobenzofuran-5-sulfonyl (Pbf) protecting groups throughout the rest of the synthesis. The remainder of the synthesis was then completed as described previously for (DvLPBaPPP)2LLA (Id.). Analytical HPLC and electrospray ionization mass spectrometry (ESI-MS) were performed to confirm the purity and identity of the (DvLPBaPPP)2LLA and (DvLPBaPPP)2LArg6AcLLA free amine SHALs.

(DvLPBaPPP)2LLA:

Starting with 50 mg (0.07 mmol) resin and 30 mg (0.07 mmol) Fmoc-D-Lys(Boc)-OH, 34 mg of (DvLPBaPPP)2LLA (Rt=7.86 min, Waters Symmetry C18, 5 μm, 4.2×150 mm column, diode array detector with a linear gradient from 95% H2O, 1% TFA to 80% acetonitrile (MeCN), 1% TFA over 12 min) was isolated as red solid after purification. ESI-MS: m/z calculated for C150H224N34O41S2 (M+3H)3+ 1075.60, found 1075.62; calculated for (M+4H)4+ 806.95, found 806.93; calculated for (M+5H)5+ 645.76, found 645.68; calculated for (M+6H)6+ 538.30, found 538.21.

(DvLPBaPPP)2LArg6AcLLA:

81 mg of (DvLPBaPPP)2LArg6AcLLA (Rt=8.30 min) starting from 90 mg (0.12 mmol) resin and 154 mg (0.29 mmol) Fmoc-D-Lys(Boc)-OH was isolated as red solid after purification. ESI-MS: m/z calculated for C194H310N60O49S2 (M+3H)3+ 1444.71, found 1444.65; calculated for (M+4H)4+ 1083.76, found 1083.78; calculated for (M+5H)5+ 867.23, found 867.18; calculated for (M+6H)6+ 722.86, found 722.78; calculated for (M+7H)7+ 619.74, found 619.62.

Attachment of DotA to SHALs.

The amine analog of the SHAL (DotA-SHAL precursor with a free epsilon amine on the first lysine) was dissolved in 500 μl anhydrous DMF and 100 μl DIEA. The hexafluorophosphate (PF6) salt of DotA N-hydroxysuccinimide (NHS) ester (933.36 g/mol, 1-1.5 equivalents) was added to the mixture as a solid. The mixture was nutated for 15 min and the reaction was monitored by analytical HPLC. Upon completion the reaction solution was diluted with 300 μl H₂O and 300 μl MeCN (both containing 1% TFA) and HPLC purified using an 85% H₂O (0.1% TFA) to 70% MeCN (0.1% TFA) gradient run over 25 min. The resulting purified DotA-SHALs were lyophilized and subsequently analyzed by analytical HPLC (Waters Symmetry C18, 5 μm, 4.2×150 mm column, diode array detector) using a linear gradient from 95% H₂O (1% TFA) to 80% MeCN (1% TFA) over 12 min) and characterized by ESI-MS.

(DvLPBaPPP)2LLDo:

Reaction of the (DvLPBaPPP)2LLA amine SHAL (6.0 mg, 1.86 μmol) with DotA NHS ester (2.0 mg, 2.14 μmol) gave 100% (Rt=7.664 min) conversion by crude analytical HPLC and yielded (DvLPBaPPP)2LLDo (8.0 mg, red solid) after purification. ESI-MS: m/z calculated for C166H250N38O48S2 (M+2H)2+ 1806.09, found 1806.22; calculated for (M+3H)3+ 1204.40, found 1204.49; calculated for (M+4H)4+ 903.55, found 903.61; calculated for (M+5H)5+ 723.04, found 723.07; calculated for (M+6H)6+ 602.70, found 602.64.

(DvLPBaPPP)2LArg6AcLLDo:

Reaction of (DvLPBaPPP)2LArg6AcLLA amine SHAL (15.0 mg, 3.46 mmol) with DotA NHS ester (5.0 mg, 5.36 μmol) gave 100% (Rt=7.70 min) conversion by crude analytical HPLC and yielded (DvLPBaPPP)2LArg6AcLLDo (12.0 mg, red solid) after purification. ESI-MS: m/z calculated for C210H336N64O56S2 (M+3H)3+ 1573.51, found 1573.54; calculated for (M+4H)4+ 1180.38, found 1180.43; calculated for (M+5H)5+ 944.51, found 944.52; calculated for (M+6H)6+ 787.26, found 787.26; calculated for (M+7H)7+ 674.94, found 674.88; calculated for (M+8H)8+ 590.69, found 590.58.

SHAL Binding to Isolated HLA-DR10 Protein

Protein binding experiments were conducted using surface plasma resonance on a Biacore 3000 (Biacore, Piscataway, N.J.) at 25° C. A research grade streptavidin immobilized chip (SA chip, Biacore) was preconditioned and normalized according to the manufacturers instructions. Biotin labeled SHALs were dissolved in DMSO and diluted in 1.05×PBS (Biacore) to a final concentration of 1×PBS pH 7.4, 5% DMSO, to match the running buffer. These SHALs were injected over the flow cell to yield a surface density of 500-1000 RU (response units). Biotin (50 μM E-Z Link Amine-PEO2-Biotin, Pierce) was injected over all cells for 1 minute at 20 μl/min as a block to reduce non-specific binding. One flow cell was used as a reference cell and a different SHAL was immobilized on each of the three other cells.

Experiments measuring the binding of HLA-DR10 to the SHALs were carried out at a flow rate of 30 μl/minute in PBS pH7.4 running buffer using all 4 flow cells. HLA-DR10 isolated from Raji cells (Rose et al. (1996) Cancer Immunol Immunother 43:26-30) was diluted in running buffer to a final concentration ranging from 10 nM to 1 μM, and a series of concentrations were run randomly in triplicate. Protein was injected for 3 minutes, allowed to dissociate for 5 minutes followed by regeneration of the surface using a 1 minute injection of 0.1% sodium dodecylsulfate (SDS) followed by a washing step with a 2 minute injection of running buffer. The data, which were double referenced by subtracting the blank reference surface and an average of 5 blank injections, were processed using the program SCRUBBER (University of Utah).

### Cell Binding Assay

Raji human Burkitt's lymphoma B-cells (American Type Culture Collection, Manassas, Va.) were maintained in RPMI-1640 media supplemented with 10% fetal bovine serum, 2 mM L-glutamine, 1 mM sodium pyruvate, 1% of a solution of nonessential amino acids (GIBCO #11140-050), and 100 units/ml of Penicillin G, 100 μg/ml Streptomycin, and 0.25 μg/ml of Amphotericin B at 37° C. in a humidified 5% CO2 atmosphere. Jurkat's cells (American Type Culture Collection, Manassas, Va.), an acute leukemia T-cell line, were maintained in the same medium with the addition of 10 mM 4-(2-hydroxyethyl)-1-piperazineethanesulfonic acid (HEPES).

A series of experiments were conducted to quantify the uptake of the 111In-labeled parent SHAL (DvLPBaPPP)2LLDo and its hexa-arginine analog (DvLPBaPPP)2LArg6AcLLDo by Raji cells, a cell line that has been previously shown to express the HLA-DR10 variant. The assays were conducted using aliquots containing 106 cells suspended in 150 μl of PBS with 5% bovine serum albumin (BSA). Aliquots of cells were treated with 0.1, 1, 5, 10 or 25 ng of 111In-labeled (DvLPBaPPP)2LLDo or (DvLPBaPPP)2LArg6AcLLDo for one hour at both 4° C. and 22° C. The tubes containing the treated cells were centrifuged to separate the cell pellet from the supernatant and the two fractions were counted in a calibrated gamma well counter to determine the amount of bound and free SHAL. Half of the cell pellets were washed twice with PBS and incubated at 22° C. for 15 min before centrifuging them again. The pooled washes and washed cell pellets were subsequently counted in the gamma well counter to assess how much of the bound SHAL may be removed by washing.

### 3-D Confocal Microscopy

SHAL binding and internalization by Raji and Jurkat's cells was assessed using the method described previously by O'Donnell et al. (O'Donnell et al. (1998) Prostate 37:91-97). Experiments were conducted comparing the binding of (DvLPBaPPP)2LLDo (the parent SHAL), its hexa-arginine analog (DvLPBaPPP)2LArg6AcLLDo, and chimeric Lym-1 (chLym-1) to Raji cells. All steps were performed at 20° C., unless indicated.

Four million Raji cells (>92% viability) in log phase growth were pelleted at 300×g, washed, and blocked for 30 min in 1 ml of 1% fraction V BSA in PBS, with constant rotation. Cells were then incubated 1 hr, at 1 million per 250 μl, with either 1% BSA in PBS or a biotinylated primary reagent: 10 nM chLym-1, 10 μM parent SHAL, or 10 μM hexa-arginine SHAL. After four washes (two in 1% BSA in PBS, two in PBS), 50 μl of the cell suspensions was applied to freshly poly-L-Lysine coated slides, and cells were allowed to adhere for 10 min in a humid chamber. Fixation and permeabilization were performed at −20° C. by using a 4 min exposure to methanol. Jurkat's cells were treated in the same manner as a control.

Slides were then washed twice in PBS and blocked in 10% fetaplex serum (Gemini Bioproducts, West Sacramento, Calif.) in PBS for 15 min and washed once in PBS. The detection reagent, Streptavidin AlexaFluor 610 (Invitrogen, Carlsbad, Calif.) was diluted 1/500 in diluent, 1000 was applied; a parafilm cover slip was layered over the solution to prevent evaporation. The slides were incubated in a humid chamber for 30 min., washed 5 times for 5 min each in PBS, and rinsed briefly in double distilled H2O. After the slides dried, cover slips were mounted with ProlongGold with 4′,6-diamidino-2-phenylindole (DAPI, Invitrogen, Carlsbad, Calif.). The slides were viewed with an Olympus FV1000 laser scanning confocal microscope and data were collected as Z-scans at 160×, with focal sections being taken 1 μm apart through the cell.

### Statistical Analysis

Data is reported as mean±SD. Statistical comparisons were based on the Wilcoxon rank sum test (Hollander and Wolfe (1973) Nonparametric statistical methods. New York: Wiley Publications), a procedure based on ranking the values of two test groups. Differences were considered statistically significant if p values were ≤0.05. The p-values were determined by the transformation Z=TAN H−lr for the correlation coefficients (CRC Handbook of Tables for Probabilities and Statistics. 2nd edn. Boca Raton, Fla.: CRC Press; 1968).

### Results

### SHAL Design and Synthesis.

Two forms of the free amine SHAL, (DvLPBaPPP)2LLA, and the hexa-arginine analog, (DvLPBaPPP)2LArg6AcLLA, were synthesized in multi-milligram amounts and purified by high performance liquid chromatography (HPLC). A biotin was attached to the ε-amino group of the terminal amine (A) on both (DvLPBaPPP)2LLA and (DvLPBaPPP)2LArg6AcLLA to produce biotinylated forms for use in cell and protein binding experiments. 1,4,7,10-tetraazacyclododecane-1,4,7,10-tetraacetic acid (DotA) was attached to both (DvLPBaPPP)2LLA and (DvLPBaPPP)2LArg6AcLLA at the same site to enable the SHALs to be labeled with 111In. The DotA SHAL (DvLPBaPPP)2LLDo and the hexa-arginine SHAL analog (DvLPBaPPP)2LArg6AcLLDo were labeled with 111In at high efficiency (>90%) with specific activities ranging from 70-85 μCi/m SHAL. Analyses of the resulting radiolabeled SHAL by HPLC and cellulose acetate electrophoresis (CAE) showed the purity of the product to be greater than 90%. D-isomers of arginine incorporated during the synthesis of the hexa-arginine sequence in (DvLPBaPPP)2LArg6AcLLDo were used to minimize the proteolytic susceptibility of the peptide. While more detailed experiments need to be carried out to adequately assess the stability of the SHAL in vivo, data obtained from one preliminary CAE experiment showed no evidence of degradation when the hexa-D-arginine SHAL analog was incubated in human plasma at 37° C. for 24 hrs (data not shown).

SHAL Affinity for HLA-DR10 Protein.

Surface Plasmon resonance binding studies were conducted with both SHALs to estimate and compare the affinity of the two SHALs for isolated HLA-DR10 protein. In a series of kinetic experiments in which biotinylated versions of the SHALs were immobilized on the surface of a streptavidin chip, the parent SHAL (DvLPBaPPP)2LLDo was observed to bind to HLA-DR10 with a Kd^{˜}21 nM. A similar Kd, ^{˜}34 nM, was obtained for the hexa-arginine containing analog (DvLPBaPPP)2LArg6A cLLDo.

Analysis of SHAL Uptake by Rah Cells Expressing HLA-DR10.

In vitro cell binding experiments were conducted using 111In-labeled parent SHAL and the hexa-arginine SHAL analog to quantify SHAL uptake and to evaluate the effect of adding the hexa-arginine tag. Uptake was assessed using Raji cells, a lymphoma cell line expressing HLA-DR10. Aliquots containing 106 cells were incubated with increasing amounts of SHAL containing 111In labeled SHAL as a tracer, and cell-associated 111In was measured before and after washing the cell pellets.

Analyses of the unwashed cell pellets showed that both the parent SHAL and the hexa-arginine SHAL are bound by Raji cells. Cell associated SHAL increased linearly with increasing SHAL concentration in the media for both SHALs and the amount of bound SHAL showed no evidence of reaching saturation over the range of SHAL concentration tested. Raji cells treated with the hexa-arginine SHAL, in contrast to those treated with the parent SHAL, bound twice as much SHAL (Table 9). A larger proportion of the hexa-arginine SHAL (67%) was also retained by the cells after washing when compared to the parent SHAL (^{˜}46%), leading to a final hexa-arginine SHAL content three times that of its parent.

SEQUENCE LISTING and the entire contents of material found in the ASCII text file titled FSP1368_Seq_listing.txt, created Oct. 15, 2020, and 5,065 bytes in size, are incorporated by reference herein.

What is claimed is:

1. A method of eliciting immune responses using synthetic antigens, the method comprising:
generating at least one substitute antigen configured for a foreign molecule;
generating a synthetic high affinity ligand molecule (SHAL) comprising at least one ligand configured to bind to an antigen presenting cell (APC) and at least one adhesion molecule specifically configured to bind with the substitute antigen;
combining the SHAL with the substitute antigen through a chemical reaction forming an antigen presenting complex by way of the at least one adhesion molecule;
introducing the antigen presenting complex to a user without an immune response to the foreign molecule; and
measuring the immune response in the user for the substitute antigen.

2. The method of claim 1 wherein the substitute antigen is generated from a toxicant, the substitute antigen being a fragment of a surface protein associated with the toxicant.

3. The method of claim 1 further comprising:
generating the substitute antigen from a pathogen; and
extracting at least a subcellular fragment from the pathogen.

4. The method of claim 3 wherein the pathogen is a virus or virus like organism.

5. The method of claim 3 wherein the pathogen is a microorganism.

6. The method of claim 1 wherein the antigen presenting complex is configured to position the substitute antigen between a peptide-binding groove of a major histocompatibility complex (MHC) Class-II molecule and a T-cell receptor protein.

7. The method of claim 1, wherein the at least one ligand is configured to bind to the APC through a peptide-binding groove of an MHC Class-II molecule.

8. The method of claim 7, wherein the at least one ligand is configured to bind to the APC through at β₁ domain of the peptide-binding groove of the MHC Class-II molecule.

9. The method of claim 8, wherein the at least one ligand comprises three ligands configured to bind to the APC through at the β₁ domain of the peptide-binding groove of the MHC Class-II molecule.

10. The method of claim 1, wherein the at least one ligand comprises at least two ligands configured to bind to the APC between an MHC Class-II molecule and the T-Cell receptor protein.

11. The method of claim 10, wherein a first ligand of the at least two ligands binds to a peptide-binding groove of the MHC Class-II molecule on a β₁ domain and a second ligand of the at least two ligands binds to the α₁ domain of the peptide-binding groove of the MHC Class-II molecule.

12. The method of claim 10, wherein a first ligand of the at least two ligands binds to a peptide-binding groove of the MHC Class-II molecule on a β₁ domain and a second ligand of the at least two ligands binds to the β₁ domain of the MHC Class-II molecule adjacent to the peptide-binding groove.

13. The method of claim 1, the SHAL comprises at least two linkage compounds specifically configured to bind with the substitute antigen.

14. The method of claim 1 further comprises generating the substitute antigen from a pathogen or a toxicant.

15. The method of claim 1, wherein measuring the immune response in the user for the substitute antigen comprises measuring a corresponding antibody for the substitute antigen in the user, and introducing a new antigen presenting complex comprising a variant substitute antigen, in response to the corresponding antibody being below a minimum threshold.

16. The method of claim 1, wherein generating the substitute antigen further comprises selecting at least one version of the substitute antigen for generating the antigen presenting complex.

17. The method of claim 1, wherein the at least one substitute antigen configured for the foreign molecule further comprises a synthetic molecule configured for a pathogen or a toxicant.
